(19) United States
(12) Patent Application Publication
(10) Pub. No.: US 2025/0154457 A1
(43) Pub. Date: May 15, 2025

(54) T-CELLS FOR ANTI-HIV CAR-T THERAPY AND METHODS OF USE

(71) Applicant: City of Hope, Duarte, CA (US)

(72) Inventors: Ryan Urak, Duarte, CA (US); Tristan Scott, Duarte, CA (US); Kevin V. Morris, Duarte, CA (US)

(21) Appl. No.: 18/548,677

(22) PCT Filed: Mar. 3, 2022
(86) PCT No.: PCT/US2022/018720

§ 371 Date: Sep. 1, 2023

Related U.S. Application Data
(60) Provisional application No. 63/156,780, filed on Mar. 4, 2021.

(51) Int. Cl. C12N 5/0783; A61K 40/11; A61K 40/31; A61K 40/46; C07K 16/10; C12N 15/113

(52) U.S. Cl. CPC C12N 5/0636; A61K 40/11; A61K 40/31; A61K 40/46; C07K 16/1063; C12N 15/113; C07K 2319/03; C12N 2310/531; C12N 2510/00

(57) ABSTRACT

Provided herein are, inter alia, a T-cell comprising a nucleic acid including a sequence encoding an anti-HIV Chimeric Antigen Receptor (CAR) and a sequence encoding a protein inhibitor. Methods for making the T-cells and for using the T-cells to treat HIV-infected patients are provided.

### CROSS-REFERENCE TO RELATED APPLICATIONS

This application claims priority to U.S. Provisional Application No. 63/156,780, filed Mar. 4, 2021, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048440-792001WO_SL_ST25.TXT, created on Mar. 3, 2022, 16,384 bytes, machine format IBM-PC, MS Windows operating system, is hereby incorporated by reference.

### BACKGROUND OF THE INVENTION

Chimeric antigen receptor (CAR) T-cell immunotherapy was previously explored for treatment of human immunodeficiency virus (HIV) infections, however lack of optimal CAR T-cell design was likely a primary reason the therapy did not show clinical benefits in subjects who were concurrently on anti-retroviral therapy (ART). Second generation CAR T-cell therapies have since produced dramatic responses in patients with B cell malignancies; thus, researchers have continued the development of CAR T-cell therapy for targeting and eliminating HIV in patients. However, there are major challenges in the field of CAR T-cell therapy that are specific for production of HIV targeting therapeutics.

Current CAR T-cells are generated by lentiviral vector transduction of patient derived T-cells ex vivo and reinfused back into the patient. One issue that arises from this methodology is that not all the T-cells are transduced with the CAR vector, thereby resulting in a population of unmodified T-cells that may cause graft vs. host disease in the patient. Further, in the case of HIV targeted CAR T-cells, the virus is able to infect the population of unmodified T-cells. What would therefore be ideal in CAR manufacturing is to have a simple and inexpensive methodology that insures that substantially of the T-cells utilized are CAR T-cells and not unmodified T-cells.

Moreover, there have been efforts to develop CAR T-cells to target HIV infected cells as a scheme to functionally curing HIV infection. One issue that arises from using T-cells to target HIV infected cells are that the CAR T-cells are also susceptible to infection.

Provided herein, inter alia, are solutions to these and other problems in the art.

### BRIEF SUMMARY OF THE INVENTION

Provided herein, inter alia, are compositions and methods for preventing HIV infection and/or HIV reactivation in anti-HIV Chimeric Antigen Receptor (CAR) T-cells. Applicants demonstrate herein that incorporation of a nucleic acid sequence encoding a CCR5 and/or a Tat/Rev inhibitor in an anti-HIV CAR construct prevents HIV infection and/or reactivation in the T-cells transduced with the construct. Provided herein, inter alia, are compositions and methods for selecting and/or enriching anti-HIV CAR T-cells in a population of cells including anti-HIV CAR transduced T-cells and non-transduced T-cells. Applicants show that selection and/or enrichment of anti-HIV CAR T-cells from populations of anti-HIV CAR transduced T-cells and untransduced T-cells increases the efficacy of anti-HIV CAR T-cells for treatment of HIV.

In an aspect is provided A T-cell including: a nucleic acid including a sequence encoding an anti-HIV Chimeric Antigen Receptor (CAR); and (i) a sequence encoding a Tat/Rev inhibitor, or (ii) a sequence encoding a CCR5 inhibitor.

In another aspect is provided a T-cell including a nucleic acid including a sequence encoding an Anti-HIV Chimeric Antigen Receptor (CAR) and a sequence encoding an HPRT inhibitor.

In an aspect is provided a pharmaceutical composition including a T-cell provided herein including embodiments thereof and a pharmaceutically-acceptable excipient.

In an aspect is provided a nucleic acid including a sequence encoding an anti-HIV Chimeric Antigen Receptor (CAR) and (i) a sequence encoding a Tat/Rev inhibitor, or (ii) a sequence encoding a CCR5 inhibitor.

In an aspect is provided a nucleic acid including a sequence encoding an anti-HIV Chimeric Antigen Receptor (CAR) and a sequence encoding a hypoxanthine phosphoribosyl transferase (HPRT) inhibitor.

In an aspect is provided a method of treating an HIV-infected subject in need thereof, the method including administering to the subject an effective amount of the T-cell provided herein including embodiments thereof.

In an aspect a method of treating an HIV-infected subject in need thereof is provided, the method including: (i) contacting a population of T-cells with a nucleic acid, thereby forming a population of transduced T-cells and non-transduced T-cells, wherein the nucleic acid includes a sequence encoding an anti-HIV Chimeric Antigen Receptor (CAR) and a sequence encoding a HPRT inhibitor; (ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells; and iii) administering to said subject an effective amount of the population of selected transduced T-cells.

In an aspect is provided a method of selecting for T-cells including: (i) contacting a population of T-cells with a nucleic acid, thereby forming a population of transduced T-cells and non-transduced T-cells, wherein the nucleic acid includes a sequence encoding an anti-HIV Chimeric Antigen Receptor (CAR) and a sequence encoding a HPRT inhibitor; and (ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells.

In an aspect is provided a T-cell including a nucleic acid encoding an anti-HIV Chimeric Antigen Receptor (CAR) and a nucleic acid encoding a Tat/Rev inhibitor. In another aspect is provided a T-cell including a nucleic acid encoding an anti-HIV CAR and a nucleic acid encoding a CCR5 inhibitor.

In an aspect is provided a T-cell, the T-cell including a nucleic acid encoding a CAR and a nucleic acid encoding an HPRT inhibitor.

In another aspect is provided a pharmaceutical composition including a T-cell provided herein including embodiments thereof and a pharmaceutically-acceptable excipient.

In an aspect a method of treating an HIV-infected subject in need thereof is provided, the method including administering to a subject an effective amount of a plurality of T-cells including a T-cell provided herein including embodiments thereof.

In another aspect a method of treating an HIV infected subject in need thereof is provided, the method including: contacting a plurality of T-cells including a T-cell provided herewith including embodiments thereof with an amount of 6-thioguanine (6TG) effective to cause hematopoietic toxicity in T-cells that are non-transduced with a nucleic acid encoding an HPRT inhibitor, thereby resulting in selection of cells transduced with the nucleic acid encoding the HPRT inhibitor; and administering to the subject an effective amount of the selected cells.

In another aspect is provided a method of selecting for CAR T-cells from a population of transduced and non-transduced T-cells, the method including: transducing a population of T-cells with a nucleic acid encoding a HPRT inhibitor and a CAR, thereby forming a population of transduced and non-transduced T-cells; and contacting the population of transduced and non-transduced T-cells with an amount of 6-thioguanine (6TG) effective to cause hematopoietic toxicity in non-transduced T-cells, thereby resulting in selection of CAR T-cells.

In an aspect is provided a method of selecting for CAR T-cells from a population of transduced and non-transduced T-cells in a subject undergoing CAR-T therapy, the method including: administering a nucleic acid encoding a HPRT inhibitor and a CAR to the subject, thereby forming the population of transduced and non-transduced T-cells; administering 6-thioguanine (6TG) to said subject in an amount effective to cause hematopoietic toxicity in non-transduced T-cells, thereby resulting in selection of CAR T-cells.

### BRIEF DESCRIPTION OF THE DRAWINGS

FIGS. 1A-1E. shRNA protection of anti-HIV CAR T-cells. FIG. 1A. Quantitative PCR analysis of CCR5 following transduction of shCCR5_shTat/Rev-CAR construct into T-cells. FIG. 1B. CCR5 expression in T-cells following transduction of shCCR5_shTat/Rev-CAR compared to non-transduced T-cells. Results indicate the construct successfully knocks down CCR5 expression. CAR or shCCR5_shTat.Rev-CAR expressing Jurkat cells were incubated with FIG. 1C. X4 or FIG. 1D. R5 tropic virus and supernatant was collected for p24 analysis. Low levels of p24 in the supernatent indicate that the shCCR5_shTat.Rev-CAR expressing T-cells inhibit viral infection. FIG. 1E. CAR (control) or shCCR5_shTat/Rev-CARs were transducted into Jurkat-cells and co-cultured with either gp120 expressing HEK cells or HEK parental cells for 24 hours. Following incubation cells were analyzed for CD69 expression by flow cytometry.

FIGS. 2A-2D. 6TG selection of HPRT repressed CAR T-cells. FIG. 2A. Quantitative PCR analysis of HPRT expression following transfection of shHPRT plasmid. Untransfected Jurkat cells were used as a control. FIG. 2B. N6 CAR (control) or shHPRT-N6 CAR were transduced into Jurkat cells and co-cultured with either gp120 expressing HEK293 cells or HEK293 parental cells for 24 hours. Following incubation, the Jurkat cells were analyzed for CD69 expression by flow cytometry. FIG. 2C. N6 CAR or shHPRT-N6 CAR expressing Jurkat cells were incubated in either 0, 1, or 3 uM of 6TG for four days and analyzed for cell counts and viability through a trypan blue assay. FIG. 2D. shHPRT-N6 CAR expressing Jurkat cells cultured in 0 or 1 uM 6TG for 5 days were analyzed for CAR expression through flow cytometry. Results show enrichment of anti-HIV CAR T-cells through incubation with 6TG.

FIGS. 3A-3F. shCCR5 shTat/Rev CAR. FIG. 3A. Schema for conventional (top panel) and antisense shCCR5 sense shTat/Rev anti-HIV CAR (bottom panel). FIGS. 3B-3C. Knockdown assay for CCR5 (FIG. 3B) and Tat/Rev (FIG. 3C) for the constructs shown in FIG. 3A. FIG. 3D. Schema for antisense shCCR5 antisense shTat/Rev anti-HIV CAR. FIGS. 3E-3F. Knockdown assay for CCR5 (FIG. 3E) and Tat/Rev (FIG. 3F) for the construct listed in FIG. 3D.

FIGS. 4A-4D. Wobble base optimization of shCCR5. FIGS. 4A-4B. Schema for original anti-CCR5 shRNA (SEQ ID NO:9) (FIG. 4A) and wobble base anti-CCR5 shRNA (SEQ ID NO:11) (FIG. 4B). FIGS. 4C-4D. Knockdown assay of CCR5 (FIG. 4C) and Tat/Rev (FIG. 4D) using the anti-HIV CAR, shCCR5 shTat/Rev anti-HIV CAR, or shCCR5w shTat/Rev anti-HIV CAR.

FIGS. 5A-5C. Wobble base CCR5 shRNAs functionality. FIG. 5A. Quantitative PCR analysis of MAGI.CCR5 cells transduced with protection CAR (shCCR5w shTat/Rev anti-HIV CAR), compared to mock-transduced cells and cells transduced with conventional anti-HIV CAR without the shRNA. Decreased levels of CCR5 mRNA indicate successful knock-down using the protection CAR. FIG. 5B. CCR5 surface expression on MAGI.CCR5 cells transduced with the protection CAR compared to CCR5 expression on mock-transduced cells and on cells transduced with anti-HIV CAR without the shRNA. FIG. 5C. CCR5 surface expression on T-cells transduced with protection CAR (shCCR5w shTat/Rev anti-HIV CAR), compared to mock-transduced cells and cells transduced with conventional anti-HIV CAR without the shRNA. Decreased levels of CCR5 are indicative of successful knockdown using anti-CCR5 shRNA.

FIGS. 6A-6B. Tat/Rev shRNA Functionality. Analysis of p24 levels in cell culture supernatant of R5-tropic (FIG. 6A) or X4-tropic (FIG. 6B) virally infected Jurkat. CCR5 cells transduced with either protection anti-HIV CAR (e.g. shCCR5w shTat/Rev anti-HIV CAR) or conventional anti-HIV CAR constructs. The results show that p24 levels are significantly lower in the supernatant of protection CAR transduced cells, indicating low levels of viral replication.

FIGS. 7A-7C. Functionality of protection anti-HIV CAR. FIGS. 7A-7B. Flow cytometry analysis showing CD69 (FIG. 7A) or CD137 (FIG. 7B) expression on protection anti-HIV CAR construct transduced T-cells when co-cultured with gp120 expressing cells. Expression levels are compared between the protection anti-HIV CAR transduced T-cells, mock-transduced T-cells and T-cells transduced with conventional anti-HIV CAR construct. Parental HEK293 cells are a negative control. FIG. 7C. Data showing cytotoxicity of protection anti-HIV CAR T-cells against 8e5 cells compared to mock-transduced T-cells or conventional anti-HIV CAR T-cells. Parental CEM cells are used as a control.

FIGS. 8A-8B. Analysis of p24 levels from HIV patient T-cells transduced with protection anti-HIV CAR lentivirus. FIG. 8A. p24 levels from cell culture supernatant and FIG. 8B. cell growth of mock-transduced, protection and conventional anti-HIV CAR throughout the course of culture. Results indicate that HIV patient T-cells transduced with protection anti-HIV CAR lentivirus inhibit viral replication compared to T-cells transduced with conventional anti-HIV CAR lentivirus and mock-transduced T-cells. Further, patient HIV cells transduced with the protection anti-HIV CAR lentivirus retains cell growth potential.

FIGS. 9A-9C. Phenotype analysis of protection anti-HIV CAR lentivirus transduced T-cells. FIG. 9A. CD4 expression level percentage over the course of cell culture for mock-transduced T-cells, conventional anti-HIV CAR and protection CAR anti-HIV CAR transduced T-cells. All T-cells were taken from HIV patients. FIG. 9B. Comparison of anti-HIV CAR expression levels for T-cells taken from healthy donors and HIV patients. Anti-HIV CAR expression was measured on day 7 and day 20. FIG. 9C. Comparison of CD62L expression levels for T-cells taken from healthy donors and HIV-infected patients. The T-cells were mock-transduced or transduced with either conventional anti-HIV CAR construct or protection anti-HIV construct.

FIGS. 10A-10B. Cytotoxic functionality of T-cells transduced with protection anti-HIV CAR construct. FIGS. 10A-10B. Cytotoxicity of protection anti-HIV CAR T-cells against 8e5 cells at 1:1 (effector to target) ratio (FIG. 10A) and 1:4 (effector to target) ratio (FIG. 10B), as compared to mock-transduced and conventional anti-HIV CAR transduced T-cells. Parental CEM cells served as a negative control. The T-cells were from an HIV patient donor.

FIGS. 11A-11B. Optimizing shHPRT CAR. FIG. 11A. Schema for the conventional anti-HIV CAR construct (top panel), U6 promoter shHPRT anti-HIV CAR construct (middle panel), and tRNA promoter shHPRT anti-HIV CAR (bottom panel). FIG. 11B. Knockdown assay against HPRT as measured by a dual luciferase knockdown assay. Constructs including the shRPTP operably linked to the U6 promoter or the tRNA promoter were tested.

FIGS. 12A-12C Functionality of tRNA and U6 promoter for expressing shHPRT in the anti-HIV CAR construct. FIGS. 12A-12B. Cell count of T-cells transduced with tRNA (FIG. 12A) or U6 (FIG. 12B) promoter linked shHPRT anti-HIV CAR lentivirus following administration of 1 M 6TG or 0 μM 6TG. FIG. 12C. Quantitative PCR analysis of HPRT from U6 promoter shHPRT anti-HIV CAR transduced and mock-transduced Jurkat cells.

FIGS. 13A-13D. Optimizing selection of T-cells transduced with enrichment anti-HIV CAR T-cell construct. FIG. 13A. Cell counts of T-cells transduced with enrichment anti-HIV CAR construct, following treatment with various concentrations of (0, 10, 20, 50, 100 nM) 6TG. FIG. 13B. Analysis of anti-HIV CAR expression following treatment with various concentration of 6TG. FIG. 13C. Treatment plan for administration of 50 nM 6TG to T-cells transduced with enrichment anti-HIV CAR construct. FIG. 13D. Flow cytometry analysis of anti-HIV CAR expression following administration of four dosages of 6TG. Mock-transduced T-cells and untreated T-cells transduced enrichment anti-HIV CAR were used as control.

FIGS. 14A-14F. Phenotype analysis of the anti-HIV CAR T-cells following enrichment. FIGS. 14A-14B. Cell count was measured after administration of GT6. Total cell count (e.g. population of cells including T-cells and enrichment anti-HIV T-cells) (FIG. 14A) and cell count of enrichment anti-HIV CAR T-cells was assessed (FIG. 14B). FIG. 14C. CD4/CD8 ratios of the enrichment anti-HIV CAR T-cells were assessed following selection with 6TG. FIG. 14D. CD45RA/CD45RO ratios were measured following 6TG selection. FIG. 14E. Measurement of CD62L expression percentage following 6TG selection. FIG. 14F. Measurement of exhaustion markers (PD-1, Tim3, Lag-3) following 6TG selection. Mock-transduced T-cells and enrichment anti-HIV CAR transduced T-cells without 6TG selection were as controls.

FIGS. 15A-15B. Functionality of enrichment anti-HIV CAR T-cells following treatment with 6TG. FIG. 15A. CD69 expression of 6TG-treated and non-treated enrichment anti-HIV CAR Jurkat cells. The cells were co-cultured with gp160-expressing HEK cells or parental HEK cells (negative control). FIG. 15B. Cytotoxicity of 6TG-treated and non-treated enrichment anti-HIV CAR T-cells. The cells were co-cultured with 8e5 cells, parental CEM cells (negative control), or mock-transduced T-cells (negative control).

FIG. 16. Selection of anti-HIV CAR T-cells with either 6TG or 6MP. T-cells were transduced with enrichment anti-HIV CAR lentivirus construct and selected with 6TG or 6MP.

### DETAILED DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. For example, the nucleic acid provided herein may be part of a vector. For example, the nucleic acid provided herein may be part of a lentiviral vector, which may be transduced into a cell. Polynucleotides useful in the methods of the disclosure may comprise natural nucleic acid sequences and variants thereof, artificial nucleic acid sequences, or a combination of such sequences.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

A “shRNA,” “short hairpin RNA,” or “small hairpin RNA” as provided herein refers to an RNA molecule including a hairpin turn that has the ability to reduce or inhibit expression of a target gene or target nucleic acid when expressed in the same cell as the target gene or target nucleic acid. shRNA expression in a cell may be accomplished by delivery of the shRNA the cell using a plasmid or vector. Typically, the shRNA is cleaved by an enzyme (i.e. Dicer) to produce an siRNA product. The siRNA may then associate with RISC, thereby allowing target recognition.

A “siRNA,” “small interfering RNA,” “small RNA,” or “RNAi” as provided herein refers to a double-stranded or single-stranded ribonucleic acid that has the ability to reduce or inhibit expression of a gene or the activity of a target nucleic acid (e.g., a single-stranded or double-stranded RNA or a single-stranded or doubles-stranded DNA) when expressed in the same cell as the gene or target gene. Where the small RNA is a double-stranded RNA, the complementary portions of the ribonucleic acid that hybridize to form the double stranded molecule typically have substantial or complete identity. In embodiments, a small RNA is a nucleic acid that has substantial or complete identity to a target RNA and forms a double stranded small RNA. In embodiments, the small RNA inhibits gene expression by interacting with a complementary cellular RNA thereby interfering with the endogenous behavior of the complementary cellular RNA. Typically, the nucleic acid is at least about 15-50 nucleotides in length (e.g., each complementary sequence of the double stranded small RNA is 15-50 nucleotides in length, and the double stranded small RNA is about 15-50 base pairs in length). The small RNAs provided herein regulate expression of a target gene or activity of a target nucleic by hybridizing to the mRNA of the gene or by hybridizing to the promoter of the target nucleic or the target nucleic acid itself. Where the small RNA hybridizes to a promoter of a gene thereby modulating the expression of said gene, the small RNA may be referred to as “antigen RNA” or “agRNA.” In embodiments, the RNA sequence provided herein is a small RNA.

A “microRNA,” “microRNA nucleic acid sequence,” “miR,” “miRNA” as used herein, refers to a nucleic acid that functions in RNA silencing and post-transcriptional regulation of gene expression. The term includes all forms of a miRNA, such as the pri-, pre-, and mature forms of the miRNA. In embodiments, microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA products. The mature miRNA is incorporated into a RNA-induced silencing complex (RISC), which recognizes target mRNAs through imperfect base pairing with the miRNA and most commonly results in translational inhibition or destabilization of the target mRNA.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is

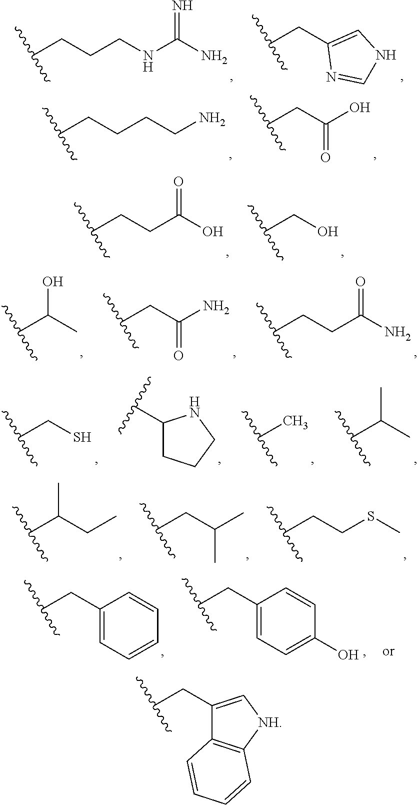

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. The preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

For specific proteins described herein, the named protein includes any of the protein's naturally occurring forms, variants or homologs that maintain activity of the protein (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “HPRT protein” or “HPRT” as used herein includes any of the recombinant or naturally-occurring forms of hypoxanthine-guanine phosphoribosyltransferase, also known as HGPRT and HGPRTase, or variants or homologs thereof that maintain HPRT activity (e.g. within at least 50%, 80%, 90%, 95%, 96%, 97%, 98%, 99% or 100% activity compared to HPRT). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring HPRT protein. In embodiments, the HPRT protein is substantially identical to the protein identified by the UniProt reference number P00492 or a variant or homolog having substantial identity thereto.

The term “Tat protein” or “Tat” as used herein includes any of the recombinant or naturally-occurring forms of Tat, also known as transactivating regulatory protein, or variants or homologs thereof that maintain Tat activity (e.g. within at least 50%, 80%, 90%, 95%, 96%, 97%, 98%, 99% or 100% activity compared to Tat). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Tat protein. In embodiments, the Tat protein is substantially identical to the protein identified by the UniProt reference number A6MI22 or a variant or homolog having substantial identity thereto.

The term “Rev protein” or “Rev” as used herein includes any of the recombinant or naturally-occurring forms of Rev, also known as ART/TRS, anti-repression transactivator, and regulator of expression of viral proteins, or variants or homologs thereof that maintain Rev activity (e.g. within at least 50%, 80%, 90%, 95%, 96%, 97%, 98%, 99% or 100% activity compared to Rev). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Rev protein. In embodiments, the Rev protein is substantially identical to the protein identified by the UniProt reference number P69718 or a variant or homolog having substantial identity thereto.

The term “CCR5 protein” or “CCR5” as used herein includes any of the recombinant or naturally-occurring forms of C—C chemokine receptor type 5, also known as C—C CKR-5, or variants or homologs thereof that maintain CCR5 activity (e.g. within at least 50%, 80%, 90%, 95%, 96%, 97%, 98%, 99% or 100% activity compared to CCR5). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CCR5 protein. In embodiments, the CCR5 protein is substantially identical to the protein identified by the UniProt reference number P51681 or a variant or homolog having substantial identity thereto.

The term “gp120 protein” or “gp120” as used herein includes any of the recombinant or naturally-occurring forms of glycoprotein 120, or variants or homologs thereof that maintain gp120 activity (e.g. within at least 50%, 80%, 90%, 95%, 96%, 97%, 98%, 99% or 100% activity compared to gp120). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gp120 protein. In embodiments, the gp120 protein is substantially identical to the protein identified by the UniProt reference number Q9IZE4 or a variant or homolog having substantial identity thereto.

The term “gp41 protein” or “gp41” as used herein includes any of the recombinant or naturally-occurring forms of gp41, also known as Env polyprotein, or variants or homologs thereof that maintain gp41 activity (e.g. within at least 50%, 80%, 90%, 95%, 96%, 97%, 98%, 99% or 100% activity compared to gp41). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gp41 protein. In embodiments, the gp41protein is substantially identical to the protein identified by the UniProt reference number Q53I19 or a variant or homolog having substantial identity thereto.

The term “gp160 protein” or “gp160” as used herein includes any of the recombinant or naturally-occurring forms of gp160, also known as Envelope glycoprotein gp160, or variants or homologs thereof that maintain gp160 activity (e.g. within at least 50%, 80%, 90%, 95%, 96%, 97%, 98%, 99% or 100% activity compared to gp160). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gp160 protein. In embodiments, the gp160 protein is substantially identical to the protein identified by the UniProt reference number P03375 or a variant or homolog having substantial identity thereto.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

As used herein, the term “construct” is intended to mean any recombinant nucleic acid molecule. In embodiments, a construct includes an expression cassette, plasmid, cosmid, virus, autonomously replicating polynucleotide molecule, phage, or linear or circular, single-stranded or double-stranded, DNA or RNA polynucleotide molecule. A construct may be derived from any source, capable of genomic integration or autonomous replication, including a nucleic acid molecule where one or more nucleic acid sequences has been linked in a functionally operative manner, e.g., operably linked.

The terms “operably linked” or “functionally linked”, are interchangeable and denote a physical or functional linkage between two or more elements, e.g., polypeptide sequences or polynucleotide sequences, which permits them to operate in their intended fashion. For example, an operable linkage between a polynucleotide of interest and a regulatory sequence (for example, a promoter) is functional link that allows for expression of the polynucleotide of interest. In this sense, the term “operably linked” refers to the positioning of a regulatory region (e.g. a promoter) and a coding sequence (e.g. polynucleotide encoding a gene editing agent, etc.) to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, operably linked elements may be contiguous or non-contiguous. In addition, in the context of a polypeptide, “operably linked” refers to a physical linkage (e.g., directly or indirectly linked) between amino acid sequences (e.g., different segments, modules, or domains) to provide for a described activity of the polypeptide. In the present disclosure, various segments, regions, or domains of the engineered antibodies disclosed herein may be operably linked to retain proper folding, processing, targeting, expression, binding, and other functional properties of the engineered antibodies in the cell. Operably linked regions, domains, and segments of the engineered antibodies of the disclosure may be contiguous or non-contiguous (e.g., linked to one another through a linker).

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a lentiviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Typically, transduction occurs by introduction of a virus or viral vector (e.g. lentiviral vector) into the cell. For example, a lentiviral vector including a nucleic acid sequence encoding an anti-HIV CAR may be transduced into a T-cell, thereby allowing expression of the anti-HIV CAR in the T-cell. For example, a transduced T-cell is a T-cell in which foreign DNA has been introduced (i.e. through introduction of a virus or viral vector into the T-cell).

As used herein, the term “promoter” refers to a sequence of DNA which proteins bind to initiate gene expression. For example, transcription factors may bind a promoter region of a gene to transcribe RNA from DNA.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be, for example, a nucleic acid as provided herein and a cell. In embodiments contacting includes, for example, allowing a nucleic acid as described herein to interact with a cell. Thus, in embodiments, contacting includes allowing a nucleic acid to interact with a cell, thereby resulting in transduced cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“T-cells” or “T lymphocytes” refer to a type of lymphocyte (a subtype of white blood cell) that plays a central role in cell-mediated immunity. They can be distinguished from other lymphocytes, such as B cells and natural killer cells, by the presence of a T-cell receptor on the cell surface. T-cells include, for example, natural killer T (NKT) cells, cytotoxic T lymphocytes (CTLs), regulatory T (Treg) cells, and T helper cells. Different types of T-cells can be distinguished by use of T-cell detection agents.

A “memory T-cell” is a T-cell that has previously encountered and responded to its cognate antigen during prior infection, encounter with cancer or previous vaccination. At a second encounter with its cognate antigen memory T-cells can reproduce (divide) to mount a faster and stronger immune response than the first time the immune system responded to the pathogen.

A “regulatory T-cell” or “suppressor T-cell” is a lymphocyte which modulates the immune system, maintains tolerance to self-antigens, and prevents autoimmune disease.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins. In embodiments, the virus is human immunodeficiency virus (HIV). HIV may infect a variety of immune cells including CD4+ T-cells, macrophages and microglial cells. HIV may gain entry into immune cells through interaction with one or more cell surface receptors expressed on the immune cells. In embodiments, HIV enters T-cells though interaction between an HIV envelope glycoprotein (i.e. gp120) and the CD4 protein on the T-cell surface. In embodiments, HIV enters T-cells though interaction with the chemokine receptor CCR5. In embodiments, HIV enters T-cells through interaction with the chemokine receptor CXCR4.

The term “HIV tropism” is used according to its plain ordinary meaning and refers to the type of cell that HIV infects. The type of cell HIV infects may be determined by the type of coreceptor on the cell that is recognized and bound by the HIV protein gp120. For example, HIV typically infects cells expressing CD4 and either the CCR5 or CXCR4 coreceptor. X4-tropic HIV may enter cells through the CD4 and the CXCR4 coreceptor expressed on the surface of target cells. Thus, “X4-tropic HIV”, also referred to as “X4 HIV”, “X4 virus” or “T-tropic HIV”, refer to HIV that can infect CD4+ T-cells and macrophages that express CD4 and CXCR4. R5-tropic HIV may enter cells through the CD4 and CCR5 coreceptor. Thus, “R5-tropic HIV”, also referred to as “R5 HIV”, “R5 virus” or “M-tropic virus”, refers to HIV that may infect CD4+ T-cells, macrophages and dendritic cells that express CD4 and CCR5. “Dual-tropic HIV” refers to HIV that may use both CCR5 and CXCR4 proteins for entry into cells.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule.

In the context of a virus, the term “replicate” includes the ability of a virus to replicate (duplicate the viral genome and packaging said genome into viral particles) in a host cell and subsequently release progeny viruses from the host cell, which results in the lysis of the host cell. For example, HIV expresses Tat protein, which allows the efficient viral transcription of HIV genes, thereby promoting viral replication. For example, HIV expresses Rev protein, which is necessary for HIV replication. Rev assists in export of unspliced and incompletely spliced mRNA. In instances, Rev positively regulates expression of structural proteins and negatively regulates expression of regulatory genes.

The term “reactivate” is used in accordance with its plain ordinary meaning and refers to the ability of a latent virus to continue transcription of viral genes and thus allow continued viral replication. Although latent viruses do not produce substantial amounts of viral particles, latent viruses are replication-competent and therefore may replicate upon appropriate activation. For example, expression of functional Tat protein may assist in reactivation of latent HIV.

The terms “multiplicity of infection” or “MOI” are used according to its plain ordinary meaning in Virology and refers to the ratio of infectious agent (e.g., poxvirus) to the target (e.g., cell) in a given area or volume. In embodiments, the area or volume is assumed to be homogenous.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to a protein-inhibitor interaction means negatively affecting (e.g. decreasing) the activity or function of the protein (i.e. Tat/Rev, CCR5, and HPRT) relative to the activity or function of the protein in the absence of the inhibitor (i.e. shRNA). In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

An “inhibitor” refers to a compound (e.g. compounds described herein) that reduces activity when compared to a control, such as absence of the compound or a compound with known inactivity. In embodiments, the inhibitor is an shRNA.

The terms “inhibitor,” “repressor” or “antagonist” or “downregulator” interchangeably refer to a substance capable of detectably decreasing the expression or activity of a given gene or protein. In embodiments, the inhibitor may decrease the expression or activity of Tat protein. In embodiments, the inhibitor may decrease the expression or activity of Rev protein. In embodiments, the inhibitor may decrease the expression or activity of CCR5 protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

Thus, the term “Tat/Rev inhibitor” as used herein refers to a compound that has the ability to decrease the expression or activity of Tat and/or Rev. In embodiments, the Tat/Rev inhibitor is an anti-Tat/Rev shRNA. As used herein, an anti-Tat/Rev shRNA refers to an shRNA that decreases or downregulates expression of the Tat and/or Rev genes. In embodiments, the anti-Tat/Rev shRNA includes the sequence of SEQ ID NO:10.

The term “CCR5 inhibitor” as used herein refers to a compound that has the ability to decrease the expression or activity of CCR5. In embodiments, the CCR5 inhibitor is an anti-CCR5 shRNA. As used herein, an anti-CCR5 shRNA refers to an shRNA that decreases or downregulates expression of the CCR5 gene. In embodiments, the anti-CCR5 shRNA includes the sequence of SEQ ID NO: 11. In embodiments, the anti-CCR5 shRNA includes the sequence of SEQ ID NO: 9.

The term “HPRT inhibitor” as used herein refers to a compound that has the ability to decrease the expression or activity of HPRT. In embodiments, the HPRT inhibitor is an anti-HPRT shRNA. As used herein, an anti-HPRT shRNA refers to an shRNA that decreases or downregulates expression of the HPRT gene. In embodiments, the anti-HPRT shRNA includes the sequence of SEQ ID NO:12.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient”, “subject” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“HIV infected subject” or “HIV patient” refers to a living organism to whom a virus has been transmitted by viral entry into the subject, and viral replication within cells of the subject. An HIV infected subject may be in any one of the phases of HIV infection (i.e. primary/acute infection, latent infection, symptomatic infection, AIDS, etc.). For example, an HIV infected subject may have a primary infection as a first phase of HIV infection. Symptoms of a primary infection may be similar to a flu infection (i.e. fever, sore throat, muscle aches, diarrhea, rash, cough, weight loss, etc.). Following the primary infection, an HIV infected subject may be in a stage of latent or chronic infection, wherein the subject may not have symptoms or mild symptoms. During this stage, the HIV virus may reproduce at low levels. Upon further replication of the virus in the HIV infected subject, other infections (i.e. oral yeast infection, shingles, pneumonia, etc.) or symptoms (i.e. fever, fatigue, swollen lymph nodes, diarrhea, weight loss, etc.) may develop in the subject. If HIV continues to replicate, and if CD4+ cells in the subject drops below 200 cells/mm³, the HIV infection has progressed to AIDS. During this stage of infection, the HIV infected subject may develop opportunistic infections (i.e. HSV-1, salmonella, yeast infection, toxoplasmosis, etc.).

The terms “disease” or “condition” refer to a state of being or health status of a patient or subject capable of being treated with the compounds or methods provided herein. The disease can be an autoimmune, inflammatory, cancer, infectious, metabolic, developmental, cardiovascular, liver, intestinal, endocrine, neurological, or other disease. In some examples, the disease is an infectious disease (e.g. an HIV infection).

The term “infection” or “infectious disease” refers to a disease or condition that can be caused by organisms such as a bacterium, virus, fungi or any other pathogenic microbial agents. In embodiments, the infectious disease is caused by a pathogenic virus. Pathogenic viruses are viruses that can infect and replicate within cells (e.g. human cells) and cause diseases. In embodiments, the infectious disease is a virus associated disease. In embodiments, the infectious disease is human immunodeficiency virus (HIV) infection. In embodiments, the infectious disease is acquired immunodeficiency syndrome (AIDS). In embodiments, the infectious disease is one or more conditions or symptoms caused by HIV infection or AIDS.

The term “associated” or “associated with” in the context of a substance or substance activity or function associated with a disease means that the disease (e.g. HIV) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. For example, administration may be intravenous infusion of a T-cell provided herein into a subject in need thereof. For example, administration may be direct injection of lentiviral vectors into a subject in need thereof. For example, administration may be direct injection of lentivial vectors into patient bone marrow. In embodiments, the administering does not include administration of any active agent other than the recited active agent.

“Co-administer” it is meant that a composition described herein is administered at the same time, just prior to, or just after the administration of one or more additional therapies. The compositions and compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). For example, the T-cells provided herein including embodiments thereof may be co-administered with a small molecule Tat/Rev inhibitor and/or a small molecule CCR5 inhibitor. The compositions of the present disclosure can be delivered transdermally, by a topical route, or formulated as applicator sticks, solutions, suspensions, emulsions, gels, creams, ointments, pastes, jellies, paints, powders, and aerosols.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### T-Cell Compositions

Provided herein, inter alia, are compositions comprising a T-cell including a nucleic acid having a sequence encoding an anti-HIV CAR and a sequence encoding a protein inhibitor (e.g. anti-CCR5 shRNA, anti-Tat/Rev shRNA, anti-HPRT shRNA, etc.). In embodiments, the protein inhibitor (e.g. anti-CCR5 shRNA, anti-Tat/Rev shRNA) is contemplated to be effective for preventing HIV infection and/or reactivation of latent HIV in the T-cell. Provided herein, inter alia, are compositions including a T-cell including a nucleic acid encoding an anti-HIV CAR and a protein inhibitor (e.g. anti-HPRT shRNA), wherein the protein inhibitor allows selection of T-cells transduced with an anti-HIV CAR encoding nucleic acid from a population of transduced and non-transduced cells.

As used herein, “protein inhibitor” refers to a compound that detectably decreases the expression, activity, or function of a protein. For example, the protein inhibitor may decrease expression of a protein (e.g. Tat/Rev, CCR5, HPRT, etc.) at the transcriptional or translational level. In embodiments, the protein inhibitor decreases protein expression or activity 10%, 20%, 30%, 40%, 50%, 60%, 70%, 80%, 90% or more in comparison to a control in the absence of the inhibitor. In certain instances, protein expression or activity is 1.5-fold, 2-fold, 3-fold, 4-fold, 5-fold, 10-fold or lower than the expression or activity in the absence of the inhibitor. The protein inhibitor may target a T-cell protein (i.e. CCR5) that facilitates viral entry into a host cell. In embodiments, the protein inhibitor is a CCR5 inhibitor. The protein inhibitor may target an HIV protein (i.e. Tat, Rev) that allows viral replication or reactivation of latent virus in host cells. In embodiments, the protein inhibitor is a Tat inhibitor. In embodiments, the protein inhibitor is a Rev inhibitor. In embodiments, the protein inhibitor inhibits Tat protein and Rev protein (e.g. a Tat/Rev inhibitor) expression. In embodiments, the protein inhibitor inhibits Tat protein and Rev protein (e.g. a Tat/Rev inhibitor) activity. Thus, in embodiments, the protein inhibitor is a Tat/Rev inhibitor. In embodiments, the protein inhibitor targets a protein (e.g. HPRT) that converts a compound (e.g. 6TG, 6-MP) to a metabolite toxic to a cell. In embodiments, the protein inhibitor is an HPRT inhibitor. Applicant demonstrates herein that the protein inhibitors provided herein do not affect function or activity of the T-cell. Applicant further demonstrates that a T-cell including one or more protein inhibitors provided herein show increased expansion potential and memory phenotype compared to a T-cell without the one or more protein inhibitors.

In embodiments, the protein inhibitor (e.g. Tat/Rev inhibitor, CCR5 inhibitor, or HPRT inhibitor) is a nucleic acid that hybridizes to a target sequence to decrease or inhibit expression of the target protein (e.g. Tat/Rev protein, CCR5 protein, or HPRT protein). Full complementarity is not necessarily required, provided there is sufficient complementarity to cause hybridization. Thus, in embodiments, the protein inhibitor is an anti-Tat/Rev nucleic acid, an anti-CCR5 nucleic acid, or an anti-HPRT nucleic acid. In embodiments, the Tat/Rev inhibitor is an anti-Tat/Rev nucleic acid. In embodiments, the CCR5 inhibitor is an anti-CCR5 nucleic acid. In embodiments, the HPRT inhibitor is an a anti-HPRT nucleic acid. Hybridization of the anti-Tat/Rev nucleic acid, anti-CCR5 nucleic acid, or anti-HPRT nucleic acid to a target sequence (e.g. DNA sequence or RNA sequence) may prevent transcription or translation of the sequence, thereby inhibiting expression of the protein. In some instances hybridization of the nucleic acid to the target sequence induces degradation of the sequence. In embodiments, the protein inhibitor is an RNA, wherein the RNA hybridizes to a target sequence to inhibit expression of a protein (e.g. Tat/Rev protein, CCR5 protein, or HPRT protein). Thus, in embodiments, the anti-Tat/Rev nucleic acid is an RNA, referred to herein as an “anti-Tat/Rev RNA”. In embodiments, the anti-CCR5 nucleic acid is an RNA, referred to herein as an “anti-CCR5 RNA”. In embodiments, the anti-HPRT nucleic acid is an RNA, referred to herein as an “anti-HPRT RNA”. In embodiments, the anti-Tat/Rev RNA, the anti-CCR5 RNA, and the anti-HPRT RNA independently include one or more modifications that enhance serum stability. In embodiments, the anti-Tat/Rev RNA, the anti-CCR5 RNA, and the anti-HPRT RNA independently include one or more modifications including a phosphothioate internucleotide linkage, a 2′-O-methyl ribonucleotide, a 2′-deoxy-2′fluoro ribonucleotide, a 2′-deoxy ribonucleotide, a universal base nucleotide, a 5-C methyl nucleotide, an inverted deoxybasic residue incorporation, or a locked nucleic acid.

In embodiments, the anti-Tat/Rev RNA, the anti-CCR5 RNA, and the anti-HPRT RNA are independently an antisense oligonucleotide (ASO), an shRNA, an siRNA, or an miRNA. In embodiments, the anti-Tat/Rev RNA is an ASO, an shRNA, an siRNA, or an miRNA. In embodiments, the anti-Tat/Rev RNA is an ASO. In embodiments, the anti-Tat/Rev RNA is an shRNA. In embodiments, the anti-Tat/Rev RNA is an siRNA. In embodiments, the anti-Tat/Rev RNA is an miRNA. In embodiments, the anti-CCR5 RNA is an ASO, an shRNA, an siRNA, or an miRNA. In embodiments, the anti-CCR5 RNA is an ASO. In embodiments, the anti-CCR5 RNA is an shRNA. In embodiments, the anti-CCR5 RNA is an siRNA. In embodiments, the anti-CCR5 RNA is an miRNA. In embodiments, the anti-HPRT RNA is an ASO, an shRNA, an siRNA, or an miRNA. In embodiments, the anti-HPRT RNA is an ASO. In embodiments, the anti-HPRT RNA is an shRNA. In embodiments, the anti-HPRT RNA is an siRNA. In embodiments, the anti-HPRT RNA is an miRNA.

Thus, in an aspect is provided a T-cell including a nucleic acid including a sequence encoding an anti-HIV Chimeric Antigen Receptor (CAR) and i) a sequence encoding a Tat/Rev inhibitor or ii) a sequence encoding a CCR5 inhibitor. In embodiments, the nucleic acid includes a sequence encoding an anti-HIV CAR and a sequence encoding a Tat/Rev inhibitor. In embodiments, the nucleic acid includes a sequence encoding an anti-HIV CAR and a sequence encoding a CCR5 inhibitor. In embodiments, the nucleic acid includes a sequence encoding an anti-HIV CAR, a sequence encoding a Tat/Rev inhibitor, and a sequence encoding a CCR5 inhibitor.

In embodiments, the sequence encoding the Tat/Rev inhibitor includes the sequence of SEQ ID NO:1. In embodiments, the sequence encoding the Tat/Rev inhibitor includes a sequence having at least 80%, 85%, 90%, 91%, 92%, 93%, 94%, 95%, 96%, 97%, 98%, or 99% sequence identity to the sequence of SEQ ID NO:1. In embodiments, the sequence encoding the Tat/Rev inhibitor includes a sequence having at least 80% sequence identity to the sequence of SEQ ID NO:1. In embodiments, the sequence encoding the Tat/Rev inhibitor includes a sequence having at least 85% sequence identity to the sequence of SEQ ID NO:1. In embodiments, the sequence encoding the Tat/Rev inhibitor includes a sequence having at least 90% sequence identity to the sequence of SEQ ID NO:1. In embodiments, the sequence encoding the Tat/Rev inhibitor includes a sequence having at least 95% sequence identity to the sequence of SEQ ID NO:1. In embodiments, the sequence encoding the Tat/Rev inhibitor is the sequence of SEQ ID NO: 1.

In embodiments, the sequence encoding the CCR5 inhibitor includes the sequence of SEQ ID NO:2. In embodiments, the sequence encoding the CCR5 inhibitor includes a sequence having at least 80%, 85%, 90%, 91%, 92%, 93%, 94%, 95%, 96%, 97%, 98%, or 99% sequence identity to the sequence of SEQ ID NO:2. In embodiments, the sequence encoding the CCR5 inhibitor includes a sequence having at least 80% sequence identity to the sequence of SEQ ID NO:2. In embodiments, the sequence encoding the CCR5 inhibitor includes a sequence having at least 85% sequence identity to the sequence of SEQ ID NO:2. In embodiments, the sequence encoding the CCR5 inhibitor includes a sequence having at least 90% sequence identity to the sequence of SEQ ID NO:2. In embodiments, the sequence encoding the CCR5 inhibitor includes a sequence having at least 95% sequence identity to the sequence of SEQ ID NO:2. In embodiments, the sequence encoding the CCR5 inhibitor is the sequence of SEQ ID NO:2. In embodiments, the sequence encoding the CCR5 inhibitor includes the sequence of SEQ ID NO:8. In embodiments, the sequence encoding the CCR5 inhibitor includes a sequence having at least 80% sequence identity to the sequence of SEQ ID NO:8. In embodiments, the sequence encoding the CCR5 inhibitor includes a sequence having at least 85% sequence identity to the sequence of SEQ ID NO:8. In embodiments, the sequence encoding the CCR5 inhibitor includes a sequence having at least 90% sequence identity to the sequence of SEQ ID NO:8. In embodiments, the sequence encoding the CCR5 inhibitor includes a sequence having at least 95% sequence identity to the sequence of SEQ ID NO:8. In embodiments, the sequence encoding the CCR5 inhibitor is the sequence of SEQ ID NO:8.

In embodiments, the anti-Tat/Rev inhibitor is an anti-Tat/Rev shRNA. In embodiments, the anti-Tat/Rev shRNA includes the sequence of SEQ ID NO:10. In embodiments, the sequence of the anti-Tat/Rev shRNA includes a sequence having at least 80%, 85%, 90%, 91%, 92%, 93%, 94%, 95%, 96%, 97%, 98%, or 99% sequence identity to the sequence of SEQ ID NO:10. In embodiments, the anti-Tat/Rev shRNA includes a sequence having at least 80% identity to the sequence of SEQ ID NO:10. In embodiments, the anti-Tat/Rev shRNA includes a sequence having at least 85% identity to the sequence of SEQ ID NO:10. In embodiments, the anti-Tat/Rev shRNA includes a sequence having at least 90% identity to the sequence of SEQ ID NO: 10. In embodiments, the anti-Tat/Rev shRNA includes a sequence having at least 95% identity to the sequence of SEQ ID NO:10. In embodiments, the anti-Tat/Rev shRNA is the sequence of SEQ ID NO: 10.

In embodiments, the anti-CCR5 inhibitor is an anti-CCR5 shRNA. In embodiments, the anti-CCR5 shRNA includes the sequence of SEQ ID NO:11. In embodiments, the sequence of the anti-CCR5 shRNA includes a sequence having at least 80%, 85%, 90%, 91%, 92%, 93%, 94%, 95%, 96%, 97%, 98%, or 99% sequence identity to the sequence of SEQ ID NO:11. In embodiments, the anti-CCR5 shRNA includes a sequence having at least 80% sequence identity to SEQ ID NO:11. In embodiments, the anti-CCR5 shRNA includes a sequence having at least 85% sequence identity to SEQ ID NO:11. In embodiments, the anti-CCR5 shRNA includes a sequence having at least 90% sequence identity to SEQ ID NO:11. In embodiments, the anti-CCR5 shRNA includes a sequence having at least 95% sequence identity to SEQ ID NO:11. In embodiments, the anti-CCR5 shRNA is the sequence of SEQ ID NO:11. In embodiments, the anti-CCR5 shRNA includes the sequence of SEQ ID NO:9. In embodiments, the anti-CCR5 shRNA includes a sequence having at least 80% sequence identity to SEQ ID NO:9. In embodiments, the anti-CCR5 shRNA includes a sequence having at least 85% sequence identity to SEQ ID NO:9. In embodiments, the anti-CCR5 shRNA includes a sequence having at least 90% sequence identity to SEQ ID NO:9. In embodiments, the anti-CCR5 shRNA includes a sequence having at least 95% sequence identity to SEQ ID NO:9. In embodiments, the anti-CCR5 shRNA is the sequence of SEQ ID NO:9.

T-cells modified with a nucleic acid encoding an anti-HIV CAR and one or more protein inhibitors may be generated by transduction of a lentiviral vector (e.g. by way of virus like particles, virus, etc.). Generally, not all T-cells are transduced with the nucleic acid, thereby resulting in a population of un-transduced T cells. Thus, in embodiments, a sequence encoding a hypoxanthine phosphoribosyl transferase (HPRT) inhibitor is included. An HPRT inhibitor causes resistance to antimetabolites of purine analogs (e.g. 6-thioguanine (6TG), mercaptopurine (6-MP)), which are typically processed by HPRT and integrated into DNA, ultimately leading to cell death. Inhibition of HPRT in T-cells thus decreases 6TG- or 6-MP-induced cell death, thereby allowing selection and/or enrichment of transduced T-cells from populations of non-transduced and transduced T-cells (e.g. T-cells including the anti-HIV CAR encoding nucleic acid). Thus, for the T-cell provided herein, in embodiments, the nucleic acid further includes a sequence encoding a HPRT inhibitor.

In embodiments, the sequence encoding the HPRT inhibitor includes the sequence of SEQ ID NO:3. In embodiments, the sequence encoding the HPRT inhibitor includes a sequence having at least 80%, 85%, 90%, 91%, 92%, 93%, 94%, 95%, 96%, 97%, 98%, or 99% sequence identity to the sequence of SEQ ID NO:3. In embodiments, the sequence encoding the HPRT inhibitor includes a sequence having at least 80% sequence identity to the sequence of SEQ ID NO:3. In embodiments, the sequence encoding the HPRT inhibitor includes a sequence having at least 85% sequence identity to the sequence of SEQ ID NO:3. In embodiments, the sequence encoding the HPRT inhibitor includes a sequence having at least 90% sequence identity to the sequence of SEQ ID NO:3. In embodiments, the sequence encoding the HPRT inhibitor includes a sequence having at least 95% sequence identity to the sequence of SEQ ID NO:3. In embodiments, the sequence encoding the HPRT inhibitor is the sequence of SEQ ID NO:3.

In embodiments, the HPRT inhibitor is an anti-HPRT shRNA. In embodiments, the anti-HPRT shRNA includes the sequence of SEQ ID NO:12. In embodiments, the anti-HPRT shRNA includes a sequence having at least 80%, 85%, 90%, 91%, 92%, 93%, 94%, 95%, 96%, 97%, 98%, or 99% sequence identity to the sequence of SEQ ID NO:12. In embodiments, the anti-HPRT shRNA includes a sequence having at least 80% sequence identity to SEQ ID NO:12. In embodiments, the anti-HPRT shRNA includes a sequence having at least 85% sequence identity to SEQ ID NO:12. In embodiments, the anti-HPRT shRNA includes a sequence having at least 90% sequence identity to SEQ ID NO:12. In embodiments, the anti-HPRT shRNA includes a sequence having at least 95% sequence identity to SEQ ID NO:12. In embodiments, the anti-HPRT shRNA is the sequence of SEQ ID NO:12.

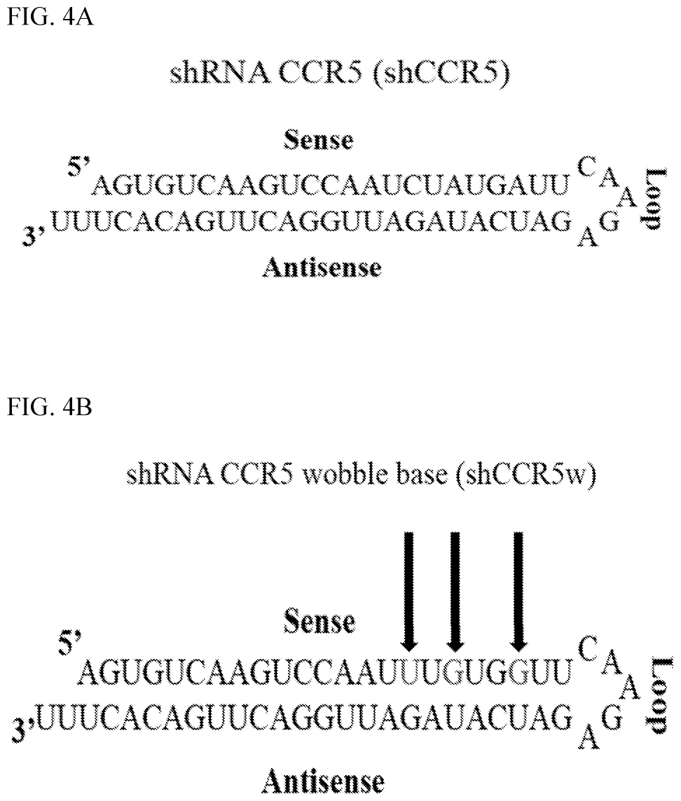

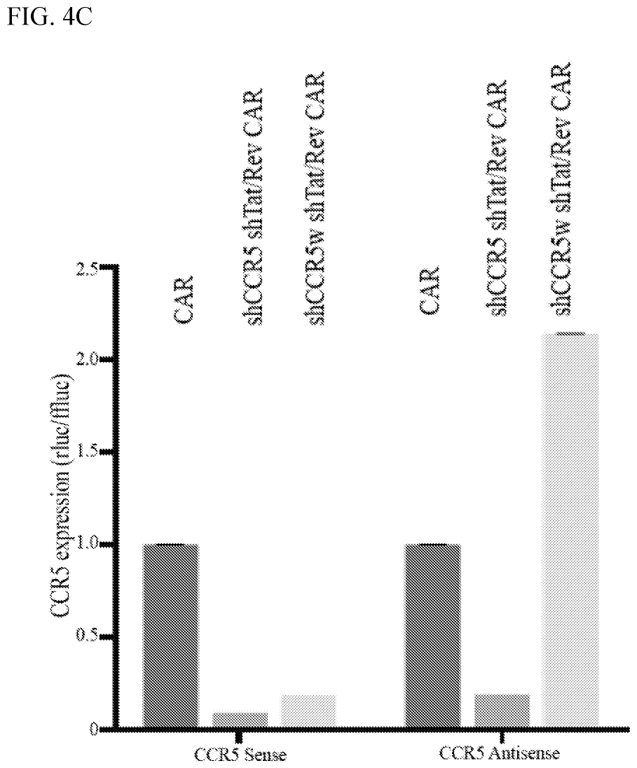

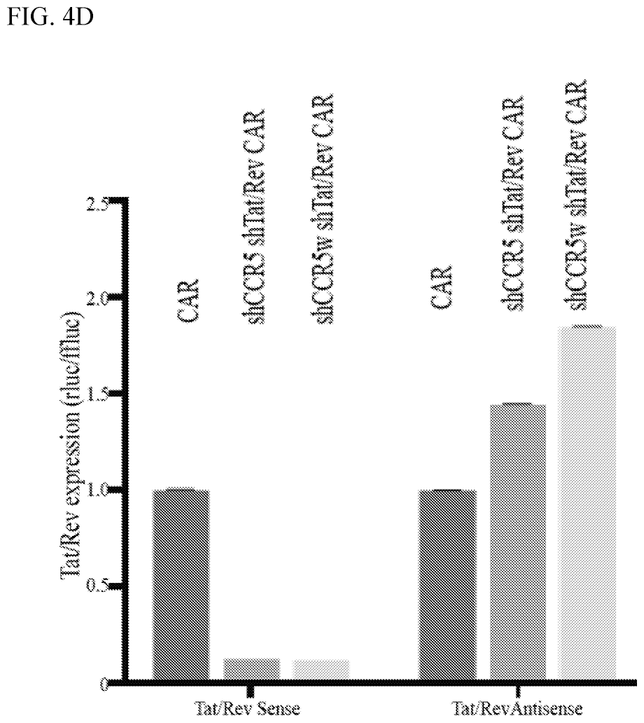

In instances, the shRNA provided herein may be self-targeting. For example, in certain instances, a strand (e.g. antisense strand) of the processed shRNA, referred to as the self-targeting strand, may hybridize to the shRNA encoding sequence. Hybridization of the self-targeting strand to the shRNA encoding sequence may thereby cause degradation of the shRNA encoding sequence. Thus, one or more wobble bases may be introduced into the self-targeting strand of the shRNA. See, for example, FIG. 4B. Introduction of one or more wobble bases into the self-targeting strand of shRNA increases selection and/or stability of the non-self-targeting strand. Thus, selection of the non-self-targeting strand may result in decreased self-targeting effects. Thus, in embodiments, one or more of the anti-Tat/Rev shRNA, the anti-CCR5 shRNA, or the anti-HPRT shRNA includes a wobble base. In embodiments, the anti-Tat/Rev shRNA includes a wobble base. In embodiments, the anti-CCR5 shRNA includes a wobble base. In embodiments, the anti-HPRT shRNA includes a wobble base. The term “wobble base” is used in accordance to its plain ordinary meaning in the biological arts and refers to base pairing between nucleotides in RNA molecules that does not follow Watson-Crick base pair rules. In embodiments, the wobble base pair is guanine-uracil (G-U), hypoxanthine-uracil (I-U), hypoxanthine-adenine (I-A), and hypoxanthine-cytosine (I-C). In embodiments, the wobble base pair is guanine-uracil (G-U).

In embodiments, the nucleic acid further includes a promoter operably linked to the sequence encoding an anti-HIV Chimeric Antigen Receptor (CAR) and the sequence encoding the Tat/Rev inhibitor. In embodiments, the nucleic acid further includes a promoter operably linked to the sequence encoding an anti-HIV Chimeric Antigen Receptor (CAR) and the sequence encoding a CCR5 inhibitor. In embodiments, the nucleic acid further includes a promoter operably linked to the sequence encoding an anti-HIV Chimeric Antigen Receptor (CAR), the sequence encoding the Tat/Rev inhibitor, and the sequence encoding a CCR5 inhibitor. In embodiments, the nucleic acid further includes a promoter operably linked to the sequence encoding an anti-HIV Chimeric Antigen Receptor (CAR), the sequence encoding the Tat/Rev inhibitor, the sequence encoding a CCR5 inhibitor, and the sequence encoding a hypoxanthine phosphoribosyl transferase (HPRT) inhibitor. In embodiments, the nucleic acid further includes a first promoter operably linked to the sequence encoding an anti-HIV Chimeric Antigen Receptor (CAR) and a second promoter operably linked to the sequence encoding the Tat/Rev inhibitor. In embodiments, the nucleic acid further includes a first promoter operably linked to the sequence encoding an anti-HIV Chimeric Antigen Receptor (CAR) and a second promoter operably linked to the sequence encoding the CCR5 inhibitor.

In embodiments, the nucleic acid further includes a first promoter operably linked to the sequence encoding an anti-HIV Chimeric Antigen Receptor (CAR), a second promoter operably linked to the sequence encoding the Tat/Rev inhibitor, and a third promoter operably linked to the sequence encoding a CCR5 inhibitor. In embodiments, the nucleic acid further includes a first promoter operably linked to the sequence encoding an anti-HIV Chimeric Antigen Receptor (CAR), a second promoter operably linked to the sequence encoding the Tat/Rev inhibitor, a third promoter operably linked to the sequence encoding a CCR5 inhibitor, and a fourth promoter operable linked to the sequence encoding a hypoxanthine phosphoribosyl transferase (HPRT) inhibitor. In embodiments, the first promoter and the second promoter are substantially the same promoter. In embodiments, the first promoter, the second promoter, and the third promoter are substantially the same promoter. In embodiments, the first promoter, the second promoter, the third promoter, and the fourth promoter are substantially the same promoter. In embodiments, the first promoter and the second promoter are different promoters. In embodiments, the first promoter, the second promoter, and the third promoter are different promoters. In embodiments, the first promoter, the second promoter, the third promoter, and the fourth promoter are different promoters.

In embodiments, the promoter is a CMV promoter. In embodiments, the promoter is a EF1α promoter. In embodiments, the promoter is a PGK promoter. In embodiments, the promoter is a CAG.SV40 promoter. In embodiments, the promoter is a Ubc promoter. In embodiments, the promoter is a Pol III promoter. In embodiments, the promoter is a U6 promoter. In embodiments, the promoter is a H1 promoter. In embodiments, the promoter is a tRNA promoter. In embodiments, the promoter is a serine tRNA promoter. In embodiments, the promoter is a lysine 3 tRNA promoter. In embodiments, the first promoter is a CMV promoter. In embodiments, the first promoter is a EF1α promoter. In embodiments, the first promoter is a PGK promoter. In embodiments, the first promoter is a CAG.SV40 promoter. In embodiments, the first promoter is a Ubc promoter. In embodiments, the first promoter is a Pol III promoter. In embodiments, the first promoter is a U6 promoter. In embodiments, the first promoter is a H1 promoter. In embodiments, the first promoter is a tRNA promoter. In embodiments, the first promoter is a serine tRNA promoter. In embodiments, the first promoter is a lysine 3 tRNA promoter. In embodiments, the second promoter is a CMV promoter. In embodiments, the second promoter is a EF1α promoter. In embodiments, the second promoter is a PGK promoter. In embodiments, the second promoter is a CAG.SV40 promoter. In embodiments, the second promoter is a Ubc promoter. In embodiments, the second promoter is a Pol III promoter. In embodiments, the second promoter is a U6 promoter. In embodiments, the second promoter is a H1 promoter. In embodiments, the second promoter is a tRNA promoter. In embodiments, the second promoter is a serine tRNA promoter. In embodiments, the second promoter is a lysine 3 tRNA promoter. In embodiments, the third promoter is a CMV promoter. In embodiments, the third promoter is a EF1α promoter. In embodiments, the third promoter is a PGK promoter. In embodiments, the third promoter is a CAG.SV40 promoter. In embodiments, the third promoter is a Ubc promoter. In embodiments, the third promoter is a Pol III promoter. In embodiments, the third promoter is a U6 promoter. In embodiments, the third promoter is a H1 promoter. In embodiments, the third promoter is a tRNA promoter. In embodiments, the third promoter is a serine tRNA promoter. In embodiments, the third promoter is a lysine 3 tRNA promoter. In embodiments, the fourth promoter is a CMV promoter. In embodiments, the fourth promoter is a EF1α promoter. In embodiments, the fourth promoter is a PGK promoter. In embodiments, the fourth promoter is a CAG.SV40 promoter. In embodiments, the fourth promoter is a Ubc promoter. In embodiments, the fourth promoter is a Pol III promoter. In embodiments, the fourth promoter is a U6 promoter. In embodiments, the fourth promoter is a H1 promoter. In embodiments, the fourth promoter is a tRNA promoter. In embodiments, the fourth promoter is a serine tRNA promoter. In embodiments, the fourth promoter is a lysine 3 tRNA promoter.

For the T-cells provided herein, in embodiments, the anti-HIV CAR includes (i) an antibody region capable of binding an HIV envelope protein, and (ii) a transmembrane domain. In embodiments, the HIV envelope protein is gp120 or gp41. In embodiments, the HIV envelope protein is gp120. In embodiments, the HIV envelope protein is gp41.

As used herein, an “anti-HIV chimeric antigen receptor” or “anti-HIV CAR” refers to a chimeric antigen receptor including an antibody region capable of binding an antigen (epitope) expressed by HIV. “Antibody region” as provided herein refers to a protein moiety capable of binding an antigen (epitope). Thus, the antibody region is an antibody within the anti-HIV CAR construct. In embodiments, the antibody region provided herein may include a domain of an antibody (e.g., a light chain variable (VL) domain or a heavy chain variable (VH) domain). In embodiments, the antibody region is a protein conjugate. A “protein conjugate” as provided herein refers to a construct consisting of more than one polypeptide, wherein the polypeptides are bound together covalently or non-covalently. In embodiments, the polypeptides of a protein conjugate are encoded by one nucleic acid molecule. In embodiments, the polypeptides of a protein conjugate are encoded by different nucleic acid molecules. In embodiments, the polypeptides are connected through a linker. In embodiments, the polypeptides are connected through a chemical linker. In embodiments, the antibody region is an scFv. The antibody region may include a light chain variable (VL) domain and/or a heavy chain variable (VH) domain. In embodiments, the antibody region includes a light chain variable (VL) domain. In embodiments, the antibody region includes a heavy chain variable (VH) domain.

Binding of the antibody region to an HIV antigen may inhibit or decrease viral entry into a host cell. For example, antibody region binding to an HIV antigen (e.g. gp41, gp120, gp160) may block interactions between the antigen and receptors on the host cell essential for infection, thereby blocking entry of the virus into the host cell. In embodiments, the HIV antigen is an HIV envelope protein (e.g. gp41, gp120, gp160). In embodiments, the HIV envelope protein is gp41, gp120, or gp160. In embodiments, the HIV envelope protein is gp41. In embodiments, the HIV envelope protein is gp120. In embodiments, the HIV envelope protein is gp160. In embodiments, the antibody region binds the CD4-binding site (CD4bs) of gp120.

In embodiments, the antibody region of the anti-HIV CAR provided herein includes an antibody domain (e.g. scFv, etc.) that specifically binds to gp120. In embodiments, the antibody region includes an anti-gp120 antibody or a fragment thereof. In embodiments, the anti-gp120 antibody is NIH-45-46 or a fragment thereof. In embodiments, the anti-gp120 antibody comprises the CDR sequences of NIH-45-46 (i.e. the sequences of CDR1, CDR2, and CDR3). In embodiments, NIH-45-46 includes a heavy chain variable domain including the sequence of SEQ ID NO:6, and a light chain variable domain including the sequence of SEQ ID NO:7. A description of the NIH-45-46 antibody and methods for its production can be found in reference Diskin, R. et al. Increasing the Potency and Breadth of an HIV Antibody by using Structure-Based Rational Design; Science. 2011 Dec. 2; 334(6060): 1289-1293; doi: 10.1126/science.1213782, which is incorporated by reference herein in its entirety and for all purposes. In embodiments, the anti-gp120 antibody is N6 or a fragment thereof. In embodiments, the anti-gp120 antibody comprises the CDR sequences of N6 (i.e. the sequences of CDR1, CDR2, and CDR3). In embodiments, N6 includes a heavy chain variable domain including the sequence of SEQ ID NO:4, and a light chain variable domain including the sequence of SEQ ID NO:5. A description of the N6 antibody and methods for its production can be found in reference Huang, J. et al. Identification of a CD4-Binding-Site Antibody to HIV that Evolved Near-Pan Neutralization Breadth; Immunity. 2016 Nov. 15; 45(5): 1108-1121. doi: 10.1016/j.immuni.2016.10.027, which is incorporated by reference herein in its entirety and for all purposes. In embodiments, the anti-gp120 antibody is PGT121 or a fragment thereof. In embodiments, the anti-gp120 antibody comprises the CDR sequences of PGT121 (i.e. the sequences of CDR1, CRD2, and CDR3). In embodiments, PGT121 includes a heavy chain variable domain including the sequence of SEQ ID NO:14, and a light chain variable domain including the sequence of SEQ ID NO:13. In embodiments, the anti-gp120 antibody is PGT128 or a fragment thereof. In embodiments, the anti-gp120 antibody comprises the CDR sequences of PGT128 (i.e. the sequences of CDR1, CDR2, and CDR3). In embodiments, PGT128 includes a heavy chain variable domain including the sequence of SEQ ID NO:16, and a light chain variable domain including the sequence of SEQ ID NO:15.

In embodiments, the antibody region of the anti-HIV CAR includes an antibody domain (e.g. scFv, etc.) that specifically binds to gp41. In embodiments, the antibody region includes an anti-gp41 antibody or a fragment thereof. In embodiments, the anti-gp41 antibody is 3BC176 or a fragment thereof. In embodiments, the anti-gp41 antibody comprises the CDR sequences of 3BC176 (i.e. the sequences of CDR1, CDR2, and CDR3). In embodiments, 3BC176 includes a heavy chain variable domain including the sequence of SEQ ID NO:18, and a light chain variable domain including the sequence of SEQ ID NO:17.

For the T-cells provided herein, in embodiments, the antibody region includes a heavy chain variable domain including the sequence of SEQ ID NO:4 and a light chain variable domain including the sequence of SEQ ID NO:5. In one embodiment, the antibody region includes the light chain variable domain of N6 antibody and a heavy chain variable domain of N6 antibody.

For the T-cells provided herein, in embodiments, the antibody region includes a heavy chain variable domain including the sequence of SEQ ID NO:6 and a light chain variable domain including the sequence of SEQ ID NO:7. In one embodiment, the antibody region includes the light chain variable domain of antibody NIH-45-46 and a heavy chain variable domain of antibody NIH-45-46.

For the T-cells provided herein, in embodiments, the antibody region includes a heavy chain variable domain including the sequence of SEQ ID NO:14 and a light chain variable domain including the sequence of SEQ ID NO:13. In one embodiment, the antibody region includes the light chain variable domain of antibody PGT121 and a heavy chain variable domain of antibody PGT121.

For the T-cells provided herein, in embodiments, the antibody region includes a heavy chain variable domain including the sequence of SEQ ID NO:16 and a light chain variable domain including the sequence of SEQ ID NO:15. In one embodiment, the antibody region includes the light chain variable domain of antibody PGT128 and a heavy chain variable domain of antibody PGT128.

For the T-cells provided herein, in embodiments, the antibody region includes a heavy chain variable domain including the sequence of SEQ ID NO:18 and a light chain variable domain including the sequence of SEQ ID NO:17. In one embodiment, the antibody region includes the light chain variable domain of antibody 3BC176 and a heavy chain variable domain of antibody 3BC176.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4 or CD3-zeta. In embodiments, the transmembrane domain is a CD-3 zeta transmembrane domain.

In embodiments, the transmembrane domain is a CD28 transmembrane domain.

The term “CD28 transmembrane domain” as provided herein includes any of the recombinant or naturally-occurring forms of the transmembrane domain of CD28, or variants or homologs thereof that maintain CD28 transmembrane domain activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, CD28 is the protein as identified by the NCBI sequence reference GI:340545506, homolog or functional fragment thereof.

In embodiments, the transmembrane domain is a CD8 transmembrane domain. The term “CD8 transmembrane domain” as provided herein includes any of the recombinant or naturally-occurring forms of the transmembrane domain of CD8, or variants or homologs thereof that maintain CD8 transmembrane domain activity (e.g. within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, CD8 is the protein as identified by the NCBI sequence reference GI:225007534, homolog or functional fragment thereof.

In embodiments, the transmembrane domain is a CD4 transmembrane domain. The term “CD4 transmembrane domain” as provided herein includes any of the recombinant or naturally-occurring forms of the transmembrane domain of CD4, or variants or homologs thereof that maintain CD4 transmembrane domain activity (e.g. within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, CD4 is the protein as identified by the NCBI sequence reference GI:303522473, homolog or functional fragment thereof.

In embodiments, the transmembrane domain is a CD3-zeta (also known as CD247) transmembrane domain. The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g. within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, CD3-zeta is the protein as identified by the NCBI sequence reference GI:166362721, homolog or functional fragment thereof.

In embodiments, the chimeric antigen receptor further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results expression by said T cell of proteins known in the art to characteristic of activated T cell (e.g., CTLA-4, PD-1, CD28, CD69). In embodiments, the intracellular T-cell signaling domain is a CD3ζ intracellular T-cell signaling domain.

In embodiments, the chimeric antigen receptor further includes an intracellular co-stimulatory T-cell signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, an ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a 4-1BB intracellular co-stimulatory signaling domain. In embodiments, the intracellular co-stimulatory signaling domain is an ICOS intracellular co-stimulatory signaling domain. In embodiments, the intracellular co-stimulatory signaling domain is an OX-40 intracellular co-stimulatory signaling domain.

The term “CTLA-4” as referred to herein includes any of the recombinant or naturally-occurring forms of the cytotoxic T-lymphocyte-associated protein 4 protein, also known as CD152 (cluster of differentiation 152), or variants or homologs thereof that maintain CTLA-4 activity (e.g. within at least 50%, 80%, 90%, 95%, 96%, 97%, 98%, 99% or 100% activity compared to CTLA-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 protein. In embodiments, the CTLA-4 protein is substantially identical to the protein identified by the UniProt reference number P16410 or a variant or homolog having substantial identity thereto.

The term “CD28” as referred to herein includes any of the recombinant or naturally-occurring forms of the Cluster of Differentiation 28 protein, or variants or homologs thereof that maintain CD28 activity (e.g. within at least 50%, 80%, 90%, 95%, 96%, 97%, 98%, 99% or 100% activity compared to CD28). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD69” as referred to herein includes any of the recombinant or naturally-occurring forms of the Cluster of Differentiation 69 protein, or variants or homologs thereof that maintain CD69 activity (e.g. within at least 50%, 80%, 90%, 95%, 96%, 97%, 98%, 99% or 100% activity compared to CD69). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD69 protein. In embodiments, the CD69 protein is substantially identical to the protein identified by the UniProt reference number Q07108 or a variant or homolog having substantial identity thereto.

The term “4-1BB” as referred to herein includes any of the recombinant or naturally-occurring forms of the 4-1BB protein, also known as tumor necrosis factor receptor superfamily member 9 (TNFRSF9), Cluster of Differentiation 137 (CD137) and induced by lymphocyte activation (ILA), or variants or homologs thereof that maintain 4-1BB activity (e.g. within at least 50%, 80%, 90%, 95%, 96%, 97%, 98%, 99% or 100% activity compared to 4-1BB). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the 4-1BB protein is substantially identical to the protein identified by the UniProt reference number Q07011 or a variant or homolog having substantial identity thereto.

Provided herein are, inter alia, T-cells including a nucleic acid including a sequence encoding an anti-HIV CAR and a sequence encoding a hypoxanthine phosphoribosyl transferase (HPRT) inhibitor. As described above, inhibiting the expression or activity of HPRT causes resistance to antimetabolites (e.g. 6TG, 6-MP), which are typically processed by HPRT and integrated into DNA, ultimately leading to cell death. Inhibition of HPRT in T-cells decreases 6TG- or 6-MP-induced cell death, thereby allowing selection and/or enrichment of desired populations of T-cells (e.g. T-cells including nucleic acid sequence encoding an anti-HIV CAR). Further, and without wishing to be bound by scientific theory, enrichment of anti-HIV CAR T-cells may decrease the anergic CAR T-cell population, where anergy may be caused by expression and/or release of antigen from HIV-infected non-transduced T-cells.

The structure of 6-thioguanine (6TG) is as follows:

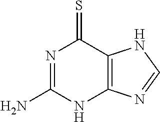 [SMILES: Nc1nc(=S)c2[nH]cnc2[nH]1]

The structure of 6-mercaptopurine (6-MP) is as follows:

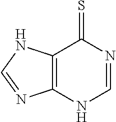

Thus, in an aspect is provided a T-cell including a nucleic acid including a sequence encoding an Anti-HIV Chimeric Antigen Receptor (CAR) and a sequence encoding an HPRT inhibitor. In embodiments, the HPRT inhibitor is an anti-HPRT shRNA. In embodiments, the anti-HPRT shRNA includes a sequence having at least 80% sequence identity to SEQ ID NO:12.

In embodiments, the nucleic acid further includes a sequence encoding a Tat/Rev inhibitor. In embodiments, the Tat/Rev inhibitor is an anti-Tat/Rev shRNA. In embodiments, the anti-Tat/Rev shRNA includes a sequence having at least 80% sequence identity to SEQ ID NO:10.

In embodiments, the nucleic acid further includes a sequence encoding a CCR5 inhibitor. In embodiments, the CCR5 inhibitor is an anti-CCR5 shRNA. In embodiments, the anti-CCR5 shRNA includes a sequence having at least 80% sequence identity to SEQ ID NO:9 or 11. In embodiments, the anti-CCR5 shRNA includes a sequence having at least 80% sequence identity to SEQ ID NO:9. In embodiments, the anti-CCR5 shRNA includes a sequence having at least 80% sequence identity to SEQ ID NO:11.

In embodiments, one or more of the anti-Tat/Rev shRNA, the anti-CCR5 shRNA, or the anti-HPRT shRNA includes a wobble base. In embodiments, the anti-Tat/Rev shRNA includes a wobble base. In embodiments, the anti-CCR5 shRNA includes a wobble base. In embodiments, the anti-HPRT shRNA includes a wobble base.

For the T-cell provided herein, in embodiments, the anti-HIV CAR includes (i) an antibody region capable of binding an HIV envelope protein; and (ii) a transmembrane domain. In embodiments, the HIV envelope protein is gp120 or gp41. In embodiments, the HIV envelope protein is gp120. In embodiments, the HIV envelope protein is gp41.

In embodiments, the antibody region includes a heavy chain variable domain including the sequence of SEQ ID NO:4 and a light chain variable domain including the sequence of SEQ ID NO:5. In embodiments, the antibody region includes a heavy chain variable domain including the sequence of SEQ ID NO:6 and a light chain variable domain including the sequence of SEQ ID NO:7.

In embodiments, the nucleic acid further includes a promoter operably linked to the sequence encoding an anti-HIV CAR and the sequence encoding the HPRT inhibitor. In embodiments, the nucleic acid further includes a promoter operably linked to the sequence encoding an anti-HIV CAR, the sequence encoding the HPRT inhibitor, and the sequence encoding the Tat/Rev inhibitor. In embodiments, the nucleic acid further includes a promoter operably linked to the sequence encoding an anti-HIV CAR, the sequence encoding the HPRT inhibitor, and the sequence encoding the CCR5 inhibitor. In embodiments, the nucleic acid further includes a first promoter operably linked to the sequence encoding an anti-HIV CAR and a second promoter operably linked to the sequence encoding the HPRT inhibitor. In embodiments, the nucleic acid further includes a first promoter operably linked to the sequence encoding an anti-HIV CAR, a second promoter operably linked to the sequence encoding the HPRT inhibitor, and a third promoter operably linked to the sequence encoding the Tat/Rev inhibitor. In embodiments, the nucleic acid further includes a first promoter operably linked to the sequence encoding an anti-HIV CAR, a second promoter operably linked to the sequence encoding the HPRT inhibitor, and a third promoter operably linked to the sequence encoding the CCR5 inhibitor. In embodiments, the nucleic acid further includes a first promoter operably linked to the sequence encoding an anti-HIV CAR, a second promoter operably linked to the sequence encoding the HPRT inhibitor, a third promoter operably linked to the sequence encoding the Tat/Rev inhibitor, and a fourth promoter operably linked to the sequence encoding the CCR5 inhibitor. In embodiments, the first promoter and the second promoter are substantially the same promoter. In embodiments, the first promoter, the second promoter, and the third promoter are substantially the same promoter. In embodiments, the first promoter, the second promoter, the third promoter, and the fourth promoter are substantially the same promoter. In embodiments, the first promoter and the second promoter are different promoters. In embodiments, the first promoter, the second promoter, and the third promoter are different promoters. In embodiments, the first promoter, the second promoter, the third promoter, and the fourth promoter are different promoters.

### Pharmaceutical Compositions

The compositions provided herein include pharmaceutical compositions including the T-cell or nucleic acid provided herein including embodiments thereof. Thus, in an aspect is provided a pharmaceutical composition including the T-cell provided herein including embodiments thereof. In another aspect is provided a pharmaceutical composition including the T-cell provided herein including embodiments thereof and a pharmaceutically acceptable excipient.

In another aspect is provided a pharmaceutical composition comprising a nucleic acid provided herein including embodiments thereof.

### Nucleic Acid Compositions

The compositions provided herein comprise nucleic acid molecules including a sequence encoding the anti-HIV Chimeric Antigen Receptor (CAR) and a sequence encoding a protein inhibitor (e.g. Tat/Rev inhibitor, CCR5 inhibitor, HPRT inhibitor) as provided herein including embodiments thereof. The anti-HIV CAR and protein inhibitor encoded by the isolated nucleic acid are described in detail throughout this application (including the description above and in the examples section). The nucleic acids can be delivered into a cell for generating anti-HIV chimeric antigen receptor (CAR) T-cells capable of expressing an anti-HIV CAR and protein inhibitor. In embodiments, the protein inhibitor (e.g. anti-CCR5 inhibitor, anti-Tat/Rev inhibitor) prevents entry of HIV into the host cell or reactivation of latent HIV in the host cell. In embodiments, the protein inhibitor (e.g. anti-HPRT inhibitor) allows for selection of the cell. Thus, in an aspect, an isolated nucleic acid including a sequence encoding an anti-HIV CAR and a sequence encoding a protein inhibitor as provided herein including embodiments thereof is provided. In embodiments, the nucleic acid includes a sequence encoding a

In another aspect is provided a nucleic acid including a sequence encoding an anti-HIV Chimeric Antigen Receptor (CAR) provided herein including embodiments thereof and i) a sequence encoding a Tat/Rev inhibitor provided herein including embodiments thereof, or ii) a sequence encoding a CCR5 inhibitor provided herein including embodiments thereof. In embodiments, the nucleic acid includes a sequence encoding a Tat/Rev inhibitor provided herein including embodiments thereof and a sequence encoding a CCR5 inhibitor provided herein including embodiments thereof. In embodiments, the Tat/Rev inhibitor is an anti-Tat/Rev shRNA. In embodiments, the CCR5 inhibitor is an anti-CCR5 shRNA. In embodiments, the nucleic acid further includes a sequence encoding a HPRT inhibitor provided herein including embodiments thereof. In embodiments, the HPRT inhibitor is an anti-HPRT shRNA.

In another aspect is provided a nucleic acid including a sequence encoding an anti-HIV Chimeric Antigen Receptor (CAR) provided herein including embodiments thereof and a sequence encoding a hypoxanthine phosphoribosyl transferase (HPRT) inhibitor provided herein including embodiments thereof. In embodiments, the HPRT inhibitor is an anti-HPRT shRNA. In embodiments, the nucleic acid further includes a sequence encoding a Tat/Rev inhibitor. In embodiments, the nucleic acid further includes a sequence encoding a CCR5 inhibitor. In embodiments, the Tat/Rev inhibitor is an anti-Tat/Rev shRNA. In embodiments, the CCR5 inhibitor is an anti-CCR5 shRNA.

In another aspect is provided an expression vector including the nucleic acid provided herein including embodiments thereof. In embodiments, the vector is a viral vector or a plasmid. In embodiments, the vector is a viral vector. In embodiments, the viral vector is a lentivirus vector.

### Methods of Treatment

The compositions provided herein are contemplated as providing effective treatments for human immunodeficiency virus (HIV). The compositions include T-cells capable of targeting and killing HIV-infected cells. The compositions further provide substantially pure populations of the T-cells provided herein, which are able to resist viral infection and reactivation. Thus, in an aspect is provided a method of treating an HIV-infected subject in need thereof, the method including administering to the subject an effective amount of the T-cell provided herein including embodiments thereof. In embodiments, the T-cell is obtained from the subject infected with HIV. In embodiments, the T-cell is not obtained from a donor who is not infected with HIV. In embodiments, the T-cell is administered intravenously.

In embodiments, the method further includes administering a small molecule inhibitor of Tat/Rev. Exemplary small molecule inhibitors of Tat/Rev include without limitation any small molecule inhibitors of Tat/Rev conventionally used and known in the art. Small molecule inhibitors are described in detail in Balachandran et al. Identification of Small Molecule Modulators of HIV-1 Tat and Rev Protein Accumulation; Retrovirology. 2017 Jan. 26; 14(1):7. doi: 10.1186/s12977-017-0330-0, which is incorporated by reference herein in its entirety and for all purposes. In embodiments, the T-cell provided herein and the small molecule inhibitor of Tat/Rev are administered simultaneously. In embodiments, the T-cell provided herein and the small molecule inhibitor of Tat/Rev are administered sequentially.

In embodiments, the method further includes administering a small molecule inhibitor of CCR5. Exemplary small molecule inhibitors of CCR5 include without limitation any small molecule inhibitors of CCR5 conventionally used and known in the art. Small molecule inhibitors are described, for example, in Qian et al. HIV Entry Inhibitors and Their Potential in HIV Therapy; Med. Res. Rev. 2009 March; 29(2): 369-393. doi: 10.1002/med.20138 and; Latinovic et al. CCR5 Inhibitors and HIV-1 Infection. J AIDS HIV Treat. 2019; 1(1): 1-5. doi:10.33696/AIDS.1.001, which are incorporated by reference herein in their entirety and for all purposes. In embodiments, the T-cell provided herein and the small molecule inhibitor of CCR5 are administered simultaneously. In embodiments, the T-cell provided herein and the small molecule inhibitor of CCR5 are administered sequentially.

For the methods provided herein, in embodiments, the T-cell is administered from an amount from about 100×10⁶ to about 600×10⁶ cells. In embodiments, the T-cell is administered from an amount from about 150×10⁶ to about 600×10⁶ cells. In embodiments, the T-cell is administered from an amount from about 200×10⁶ to about 600×10⁶ cells. In embodiments, the T-cell is administered from an amount from about 250×10⁶ to about 600×10⁶ cells. In embodiments, the T-cell is administered from an amount from about 300×10⁶ to about 600×10⁶ cells. In embodiments, the T-cell is administered from an amount from about 350×10⁶ to about 600×10⁶ cells. In embodiments, the T-cell is administered from an amount from about 400×10⁶ to about 600×10⁶ cells. In embodiments, the T-cell is administered from an amount from about 450×10⁶ to about 600×10⁶ cells. In embodiments, the T-cell is administered from an amount from about 500×10⁶ to about 600×10⁶ cells. In embodiments, the T-cell is administered from an amount from about 550×10⁶ to about 600×10⁶ cells.

For the methods provided herein, in embodiments, the T-cell is administered from an amount from about 100×10⁶ to about 550×10⁶ cells. In embodiments, the T-cell is administered from an amount from about 100×10⁶ to about 500×10⁶ cells. In embodiments, the T-cell is administered from an amount from about 100×10⁶ to about 450×10⁶ cells. In embodiments, the T-cell is administered from an amount from about 100×10⁶ to about 400×10⁶ cells. In embodiments, the T-cell is administered from an amount from about 100×10⁶ to about 350×10⁶ cells. In embodiments, the T-cell is administered from an amount from about 100×10⁶ to about 300×10⁶ cells. In embodiments, the T-cell is administered from an amount from about 100×10⁶ to about 250×10⁶ cells. In embodiments, the T-cell is administered from an amount from about 100×10⁶ to about 200×10⁶ cells. In embodiments, the T-cell is administered from an amount from about 100×10⁶ to about 150×10⁶ cells. In embodiments, the T-cell is administered from an amount of about 100×10⁶ cells, 150×10⁶ cells, 200×10⁶ cells, 250×10⁶ cells, 300×10⁶ cells, 350×10⁶ cells, 400×10⁶ cells, 450×10⁶ cells, 500×10⁶ cells, 550×10⁶ cells, or 600×10⁶ cells.

In embodiments, the T-cell is administered from an amount of about 0.1×10⁸ cells/kg to about 3×10⁸ cells/kg. In embodiments, the T-cell is administered from an amount of about 0.2×10⁸ cells/kg to about 3×10⁸ cells/kg. In embodiments, the T-cell is administered from an amount of about 0.3×10⁸ cells/kg to about 3×10⁸ cells/kg. In embodiments, the T-cell is administered from an amount of about 0.4×10⁸ cells/kg to about 3×10⁸ cells/kg. In embodiments, the T-cell is administered from an amount of about 0.5×10⁸ cells/kg to about 3×10⁸ cells/kg. In embodiments, the T-cell is administered from an amount of about 0.6×10⁸ cells/kg to about 3×10⁸ cells/kg. In embodiments, the T-cell is administered from an amount of about 0.7×10⁸ cells/kg to about 3×10⁸ cells/kg. In embodiments, the T-cell is administered from an amount of about 0.8×10⁸ cells/kg to about 3×10⁸ cells/kg. In embodiments, the T-cell is administered from an amount of about 0.9×10⁸ cells/kg to about 3×10⁸ cells/kg. In embodiments, the T-cell is administered from an amount of about 1×10⁸ cells/kg to about 3×10⁸ cells/kg. In embodiments, the T-cell is administered from an amount of about 1.1×10⁸ cells/kg to about 3×10⁸ cells/kg. In embodiments, the T-cell is administered from an amount of about 1.2×10⁸ cells/kg to about 3×10⁸ cells/kg. In embodiments, the T-cell is administered from an amount of about 1.3×10⁸ cells/kg to about 3×10⁸ cells/kg. In embodiments, the T-cell is administered from an amount of about 1.4×10⁸ cells/kg to about 3×10⁸ cells/kg. In embodiments, the T-cell is administered from an amount of about 1.5×10⁸ cells/kg to about 3×10⁸ cells/kg. In embodiments, the T-cell is administered from an amount of about 1.6×10⁸ cells/kg to about 3×10⁸ cells/kg. In embodiments, the T-cell is administered from an amount of about 1.7×10⁸ cells/kg to about 3×10⁸ cells/kg. In embodiments, the T-cell is administered from an amount of about 1.8×10⁸ cells/kg to about 3×10⁸ cells/kg. In embodiments, the T-cell is administered from an amount of about 1.9×10⁸ cells/kg to about 3×10⁸ cells/kg. In embodiments, the T-cell is administered from an amount of about 2×10⁸ cells/kg to about 3×10⁸ cells/kg. In embodiments, the T-cell is administered from an amount of about 2.1×10⁸ cells/kg to about 3×10⁸ cells/kg. In embodiments, the T-cell is administered from an amount of about 2.2×10⁸ cells/kg to about 3×10⁸ cells/kg. In embodiments, the T-cell is administered from an amount of about 2.3×10⁸ cells/kg to about 3×10⁸ cells/kg. In embodiments, the T-cell is administered from an amount of about 2.4×10⁸ cells/kg to about 3×10⁸ cells/kg. In embodiments, the T-cell is administered from an amount of about 2.5×10⁸ cells/kg to about 3×10⁸ cells/kg. In embodiments, the T-cell is administered from an amount of about 2.6×10⁸ cells/kg to about 3×10⁸ cells/kg. In embodiments, the T-cell is administered from an amount of about 2.7×10⁸ cells/kg to about 3×10⁸ cells/kg. In embodiments, the T-cell is administered from an amount of about 2.8×10⁸ cells/kg to about 3×10⁸ cells/kg. In embodiments, the T-cell is administered from an amount of about 2.9×10⁸ cells/kg to about 3×10⁸ cells/kg.

In embodiments, the T-cell is administered from an amount of about 0.1×10⁸ cells/kg to about 2.9×10⁸ cells/kg. In embodiments, the T-cell is administered from an amount of about 0.1×10⁸ cells/kg to about 2.8×10⁸ cells/kg. In embodiments, the T-cell is administered from an amount of about 0.1×10⁸ cells/kg to about 2.7×10⁸ cells/kg. In embodiments, the T-cell is administered from an amount of about 0.1×10⁸ cells/kg to about 2.6×10⁸ cells/kg. In embodiments, the T-cell is administered from an amount of about 0.1×10⁸ cells/kg to about 2.5×10⁸ cells/kg. In embodiments, the T-cell is administered from an amount of about 0.1×10⁸ cells/kg to about 2.4×10⁸ cells/kg. In embodiments, the T-cell is administered from an amount of about 0.1×10⁸ cells/kg to about 2.3×10⁸ cells/kg. In embodiments, the T-cell is administered from an amount of about 0.1×10⁸ cells/kg to about 2.2×10⁸ cells/kg. In embodiments, the T-cell is administered from an amount of about 0.1×10⁸ cells/kg to about 2.1×10⁸ cells/kg. In embodiments, the T-cell is administered from an amount of about 0.1×10⁸ cells/kg to about 2×10⁸ cells/kg. In embodiments, the T-cell is administered from an amount of about 0.1×10⁸ cells/kg to about 1.9×10⁸ cells/kg. In embodiments, the T-cell is administered from an amount of about 0.1×10⁸ cells/kg to about 1.8×10⁸ cells/kg. In embodiments, the T-cell is administered from an amount of about 0.1×10⁸ cells/kg to about 1.7×10⁸ cells/kg. In embodiments, the T-cell is administered from an amount of about 0.1×10⁸ cells/kg to about 1.6×10⁸ cells/kg. In embodiments, the T-cell is administered from an amount of about 0.1×10⁸ cells/kg to about 1.5×10⁸ cells/kg. In embodiments, the T-cell is administered from an amount of about 0.1×10⁸ cells/kg to about 1.4×10⁸ cells/kg. In embodiments, the T-cell is administered from an amount of about 0.1×10⁸ cells/kg to about 1.3×10⁸ cells/kg. In embodiments, the T-cell is administered from an amount of about 0.1×10⁸ cells/kg to about 1.2×10⁸ cells/kg. In embodiments, the T-cell is administered from an amount of about 0.1×10⁸ cells/kg to about 1.1×10⁸ cells/kg. In embodiments, the T-cell is administered from an amount of about 0.1×10⁸ cells/kg to about 1×10⁸ cells/kg. In embodiments, the T-cell is administered from an amount of about 0.1×10⁸ cells/kg to about 0.9×10⁸ cells/kg. In embodiments, the T-cell is administered from an amount of about 0.1×10⁸ cells/kg to about 0.8×10⁸ cells/kg. In embodiments, the T-cell is administered from an amount of about 0.1×10⁸ cells/kg to about 0.7×10⁸ cells/kg. In embodiments, the T-cell is administered from an amount of about 0.1×10⁸ cells/kg to about 0.6×10⁸ cells/kg. In embodiments, the T-cell is administered from an amount of about 0.1×10⁸ cells/kg to about 0.5×10⁸ cells/kg. In embodiments, the T-cell is administered from an amount of about 0.1×10⁸ cells/kg to about 0.4×10⁸ cells/kg. In embodiments, the T-cell is administered from an amount of about 0.1×10⁸ cells/kg to about 0.3×10⁸ cells/kg. In embodiments, the T-cell is administered from an amount of about 0.1×10⁸ cells/kg to about 0.2×10⁸ cells/kg. In embodiments, the T-cell is administered at an amount of about 0.1×10⁸ cells/kg, 0.2×10⁸ cells/kg, 0.3×10⁸ cells/kg, 0.4×10⁸ cells/kg, 0.5×10⁸ cells/kg, 0.6×10⁸ cells/kg, 0.7×10⁸ cells/kg, 0.8×10⁸ cells/kg, 0.9×10⁸ cells/kg, 1×10⁸ cells/kg, 1.1×10⁸ cells/kg, 1.2×10⁸ cells/kg, 1.3×10⁸ cells/kg, 1.4×10⁸ cells/kg, 1.5×10⁸ cells/kg, 1.6×10⁸ cells/kg, 1.7×10⁸ cells/kg, 1.8×10⁸ cells/kg, 1.9×10⁸ cells/kg, 2×10⁸ cells/kg, 2.1×10⁸ cells/kg, 2.2×10⁸ cells/kg, 2.3×10⁸ cells/kg, 2.4×10⁸ cells/kg, 2.5×10⁸ cells/kg, 2.6×10⁸ cells/kg, 2.7×10⁸ cells/kg, 2.8×10⁸ cells/kg, 2.9×10⁸ cells/kg, or 3×10⁸ cells/kg.

In another aspect is provided a method of treating an HIV-infected subject in need thereof, the method including: (i) contacting a population of T-cells with a nucleic acid, thereby forming a population of transduced T-cells and non-transduced T-cells, wherein the nucleic acid includes a sequence encoding an anti-HIV Chimeric Antigen Receptor (CAR) and a sequence encoding a HPRT inhibitor; (ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells; and (ii) administering to the subject an effective amount of the population of selected transduced T-cells. In embodiments, the effective amount of 6TG or 6-MP is an amount effective to cause cell death in T-cells that do not include the sequence encoding an HPRT inhibitor. The population of transduced T-cells and non-transduced T-cells comprise T-cells including the nucleic acid having the sequence encoding the anti-HIV CAR and the sequence encoding the HPRT inhibitor (e.g. transduced T-cells) and T-cells that do not comprise the nucleic acid (e.g. non-transduced T-cells). In embodiments, the population of transduced and non-transduced T-cells is due to incomplete transduction. In embodiments, the population of transduced and non-transduced T-cells is due to transduction efficiencies below 100%.

In embodiments, the population of T-cells is contacted with the amount of 6TG or 6-MP effective to cause cell death in the non-transduced T-cells (e.g. T-cells that do not include the sequence encoding an HPRT inhibitor) a plurality of times. In embodiments, the population of T-cells is contacted with the effective amount of 6TG a plurality of times. In embodiments, the population of T-cells is contacted with the effective amount of 6TG two times. In embodiments, the population of T-cells is contacted with the effective amount of 6TG three times. In embodiments, the population of T-cells is contacted with the effective amount of 6TG four times. In embodiments, the population of T-cells is contacted with the effective amount of 6TG five times. In embodiments, the population of T-cells is contacted with the effective amount of 6TG six times. In embodiments, the population of T-cells is contacted with the effective amount of 6TG seven times. In embodiments, the population of T-cells is contacted with the effective amount of 6TG eight times.

In embodiments, the population of T-cells is contacted with the effective amount of 6TG once every 6 hours. In embodiments, the population of T-cells is contacted with the effective amount of 6TG once every 12 hours. In embodiments, the population of T-cells is contacted with the effective amount of 6TG once every 18 hours. In embodiments, the population of T-cells is contacted with the effective amount of 6TG once every 24 hours. In embodiments, the population of T-cells is contacted with the effective amount of 6TG once every 30 hours. In embodiments, the population of T-cells is contacted with the effective amount of 6TG once every 36 hours. In embodiments, the population of T-cells is contacted with the effective amount of 6TG once every 42 hours. In embodiments, the population of T-cells is contacted with the effective amount of 6TG once every 48 hours.

In embodiments, the population of T-cells is contacted with the effective amount of 6-MP a plurality of times effective to cause cell death in the non-transduced T-cells. In embodiments, the population of T-cells is contacted with the effective amount of 6-MP two times. In embodiments, the population of T-cells is contacted with the effective amount of 6-MP three times. In embodiments, the population of T-cells is contacted with the effective amount of 6-MP four times. In embodiments, the population of T-cells is contacted with the effective amount of 6-MP five times. In embodiments, the population of T-cells is contacted with the effective amount of 6-MP six times. In embodiments, the population of T-cells is contacted with the effective amount of 6-MP seven times. In embodiments, the population of T-cells is contacted with the effective amount of 6-MP eight times.

In embodiments, the population of T-cells is contacted with the effective amount of 6-MP once every 6 hours. In embodiments, the population of T-cells is contacted with the effective amount of 6-MP once every 12 hours. In embodiments, the population of T-cells is contacted with the effective amount of 6-MP once every 18 hours. In embodiments, the population of T-cells is contacted with the effective amount of 6-MP once every 24 hours. In embodiments, the population of T-cells is contacted with the effective amount of 6-MP once every 30 hours. In embodiments, the population of T-cells is contacted with the effective amount of 6-MP once every 36 hours. In embodiments, the population of T-cells is contacted with the effective amount of 6-MP once every 42 hours. In embodiments, the population of T-cells is contacted with the effective amount of 6-MP once every 48 hours.

In embodiments, the amount of 6TG effective to cause cell death in non-transduced T-cells is from about 5 nM to about 1255 nM. In embodiments, the effective amount of 6TG is from about 55 nM to about 1255 nM In embodiments, the effective amount of 6TG is from about 55 nM to about 1255 nM. In embodiments, the effective amount of 6TG is from about 105 nM to about 1255 nM. In embodiments, the effective amount of 6TG is from about 155 nM to about 1255 nM. In embodiments, the effective amount of 6TG is from about 205 nM to about 1255 nM. In embodiments, the effective amount of 6TG is from about 255 nM to about 1255 nM. In embodiments, the effective amount of 6TG is from about 305 nM to about 1255 nM. In embodiments, the effective amount of 6TG is from about 355 nM to about 1255 nM. In embodiments, the effective amount of 6TG is from about 405 nM to about 1255 nM. In embodiments, the effective amount of 6TG is from about 455 nM to about 1255 nM. In embodiments, the effective amount of 6TG is from about 505 nM to about 1255 nM. In embodiments, the effective amount of 6TG is from about 555 nM to about 1255 nM. In embodiments, the effective amount of 6TG is from about 605 nM to about 1255 nM. In embodiments, the effective amount of 6TG is from about 655 nM to about 1255 nM. In embodiments, the effective amount of 6TG is from about 705 nM to about 1255 nM. In embodiments, the effective amount of 6TG is from about 755 nM to about 1255 nM. In embodiments, the effective amount of 6TG is from about 805 nM to about 1255 nM. In embodiments, the effective amount of 6TG is from about 855 nM to about 1255 nM. In embodiments, the effective amount of 6TG is from about 905 nM to about 1255 nM. In embodiments, the effective amount of 6TG is from about 955 nM to about 1255 nM. In embodiments, the effective amount of 6TG is from about 1005 nM to about 1255 nM. In embodiments, the effective amount of 6TG is from about 1055 nM to about 1255 nM. In embodiments, the effective amount of 6TG is from about 1105 nM to about 1255 nM. In embodiments, the effective amount of 6TG is from about 1155 nM to about 1255 nM. In embodiments, the effective amount of 6TG is from about 1205 nM to about 1255 nM. In embodiments, the effective amount of 6TG is from about 1255 nM to about 1255 nM.

In embodiments, the amount of 6TG effective to cause cell death in non-transduced T-cells is from about 5 nM to about 1205 nM. In embodiments, the effective amount of 6TG is from about 5 nM to about 1055 nM. In embodiments, the effective amount of 6TG is from about 5 nM to about 1105 nM. In embodiments, the effective amount of 6TG is from about 5 nM to about 1055 nM. In embodiments, the effective amount of 6TG is from about 5 nM to about 1005 nM. In embodiments, the effective amount of 6TG is from about 5 nM to about 955 nM. In embodiments, the effective amount of 6TG is from about 5 nM to about 905 nM. In embodiments, the effective amount of 6TG is from about 5 nM to about 855 nM. In embodiments, the effective amount of 6TG is from about 5 nM to about 805 nM. In embodiments, the effective amount of 6TG is from about 5 nM to about 755 nM. In embodiments, the effective amount of 6TG is from about 5 nM to about 705 nM. In embodiments, the effective amount of 6TG is from about 5 nM to about 655 nM. In embodiments, the effective amount of 6TG is from about 5 nM to about 605 nM. In embodiments, the effective amount of 6TG is from about 5 nM to about 555 nM. In embodiments, the effective amount of 6TG is from about 5 nM to about 505 nM. In embodiments, the effective amount of 6TG is from about 5 nM to about 455 nM. In embodiments, the effective amount of 6TG is from about 5 nM to about 405 nM. In embodiments, the effective amount of 6TG is from about 5 nM to about 355 nM. In embodiments, the effective amount of 6TG is from about 5 nM to about 305 nM. In embodiments, the effective amount of 6TG is from about 5 nM to about 255 nM. In embodiments, the effective amount of 6TG is from about 5 nM to about 205 nM. In embodiments, the effective amount of 6TG is from about 5 nM to about 155 nM. In embodiments, the effective amount of 6TG is from about 5 nM to about 105 nM. In embodiments, the effective amount of 6TG is from about 5 nM to about 55 nM. In embodiments, the effective amount of 6TG is about 5 nM, 55 nM, 105 nM, 155 nM, 205 nM, 255 nM, 305 nM, 355 nM, 405 nM, 455 nM, 505 nM, 555 nM, 605 nM, 655 nM, 705 nM, 755 nM, 805 nM, 855 nM, 905 nM, 955 nM, 1005 nM, 1055 nM, 1105 nM, 1155 nM, 1205 nM, or 1255 nM. In embodiments, the effective amount of 6TG is about 1000 nM. In embodiments, the effective amount of 6TG is 1000 nM.

In embodiments, the amount of 6TG effective to cause cell death in non-transduced T-cells is from about 5 nM to about 120 nM. In embodiments, the effective amount of 6TG is from about 10 nM to about 120 nM. In embodiments, the effective amount of 6TG is from about 15 nM to about 120 nM. In embodiments, the effective amount of 6TG is from about 20 nM to about 120 nM. In embodiments, the effective amount of 6TG is from about 25 nM to about 120 nM. In embodiments, the effective amount of 6TG is from about 30 nM to about 120 nM. In embodiments, the effective amount of 6TG is from about 35 nM to about 120 nM. In embodiments, the effective amount of 6TG is from about 40 nM to about 120 nM. In embodiments, the effective amount of 6TG is from about 45 nM to about 120 nM. In embodiments, the effective amount of 6TG is from about 50 nM to about 120 nM. In embodiments, the effective amount of 6TG is from about 55 nM to about 120 nM. In embodiments, the effective amount of 6TG is from about 60 nM to about 120 nM. In embodiments, the effective amount of 6TG is from about 65 nM to about 120 nM. In embodiments, the effective amount of 6TG is from about 70 nM to about 120 nM. In embodiments, the effective amount of 6TG is from about 75 nM to about 120 nM. In embodiments, the effective amount of 6TG is from about 80 nM to about 120 nM. In embodiments, the effective amount of 6TG is from about 85 nM to about 120 nM. In embodiments, the effective amount of 6TG is from about 90 nM to about 120 nM. In embodiments, the effective amount of 6TG is from about 95 nM to about 120 nM. In embodiments, the effective amount of 6TG is from about 100 nM to about 120 nM. In embodiments, the effective amount of 6TG is from about 105 nM to about 120 nM. In embodiments, the effective amount of 6TG is from about 110 nM to about 120 nM. In embodiments, the effective amount of 6TG is from about 115 nM to about 120 nM.

In embodiments, the amount of 6TG effective to cause cell death in non-transduced T-cells is from about 5 nM to about 115 nM. In embodiments, the effective amount of 6TG is from about 5 nM to about 110 nM. In embodiments, the effective amount of 6TG is from about 5 nM to about 105 nM. In embodiments, the effective amount of 6TG is from about 5 nM to about 100 nM. In embodiments, the effective amount of 6TG is from about 5 nM to about 95 nM. In embodiments, the effective amount of 6TG is from about 5 nM to about 90 nM. In embodiments, the effective amount of 6TG is from about 5 nM to about 85 nM. In embodiments, the effective amount of 6TG is from about 5 nM to about 80 nM. In embodiments, the effective amount of 6TG is from about 5 nM to about 75 nM. In embodiments, the effective amount of 6TG is from about 5 nM to about 70 nM. In embodiments, the effective amount of 6TG is from about 5 nM to about 65 nM. In embodiments, the effective amount of 6TG is from about 5 nM to about 60 nM. In embodiments, the effective amount of 6TG is from about 5 nM to about 55 nM. In embodiments, the effective amount of 6TG is from about 5 nM to about 50 nM. In embodiments, the effective amount of 6TG is from about 5 nM to about 45 nM. In embodiments, the effective amount of 6TG is from about 5 nM to about 40 nM. In embodiments, the effective amount of 6TG is from about 5 nM to about 35 nM. In embodiments, the effective amount of 6TG is from about 5 nM to about 30 nM. In embodiments, the effective amount of 6TG is from about 5 nM to about 25 nM. In embodiments, the effective amount of 6TG is from about 5 nM to about 20 nM. In embodiments, the effective amount of 6TG is from about 5 nM to about 15 nM. In embodiments, the effective amount of 6TG is from about 5 nM to about 10 nM. In embodiments, the effective amount of 6TG is about 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, or 120 nM. In embodiments, the effective amount of 6TG is about 10 nM. In embodiments, the effective amount of 6TG is 10 nM. In embodiments, the effective amount of 6TG is about 20 nM. In embodiments, the effective amount of 6TG is 20 nM. In embodiments, the effective amount of 6TG is about 50 nM. In embodiments, the effective amount of 6TG is 50 nM.

In embodiments, the amount of 6-MP effective to cause cell death in non-transduced T-cells is from about 5 nM to about 1255 nM. In embodiments, the effective amount of 6-MP is from about 55 nM to about 1255 nM In embodiments, the effective amount of 6-MP is from about 55 nM to about 1255 nM. In embodiments, the effective amount of 6-MP is from about 105 nM to about 1255 nM. In embodiments, the effective amount of 6-MP is from about 155 nM to about 1255 nM. In embodiments, the effective amount of 6-MP is from about 205 nM to about 1255 nM. In embodiments, the effective amount of 6-MP is from about 255 nM to about 1255 nM. In embodiments, the effective amount of 6-MP is from about 305 nM to about 1255 nM. In embodiments, the effective amount of 6-MP is from about 355 nM to about 1255 nM. In embodiments, the effective amount of 6-MP is from about 405 nM to about 1255 nM. In embodiments, the effective amount of 6-MP is from about 455 nM to about 1255 nM. In embodiments, the effective amount of 6-MP is from about 505 nM to about 1255 nM. In embodiments, the effective amount of 6-MP is from about 555 nM to about 1255 nM. In embodiments, the effective amount of 6-MP is from about 605 nM to about 1255 nM. In embodiments, the effective amount of 6-MP is from about 655 nM to about 1255 nM. In embodiments, the effective amount of 6-MP is from about 705 nM to about 1255 nM. In embodiments, the effective amount of 6-MP is from about 755 nM to about 1255 nM. In embodiments, the effective amount of 6-MP is from about 805 nM to about 1255 nM. In embodiments, the effective amount of 6-MP is from about 855 nM to about 1255 nM. In embodiments, the effective amount of 6-MP is from about 905 nM to about 1255 nM. In embodiments, the effective amount of 6-MP is from about 955 nM to about 1255 nM. In embodiments, the effective amount of 6-MP is from about 1005 nM to about 1255 nM. In embodiments, the effective amount of 6-MP is from about 1055 nM to about 1255 nM. In embodiments, the effective amount of 6-MP is from about 1105 nM to about 1255 nM. In embodiments, the effective amount of 6-MP is from about 1155 nM to about 1255 nM. In embodiments, the effective amount of 6-MP is from about 1205 nM to about 1255 nM.

In embodiments, the amount of 6-MP effective to cause cell death in non-transduced T-cells is from about 5 nM to about 1205 nM. In embodiments, the effective amount of 6-MP is from about 5 nM to about 1055 nM. In embodiments, the effective amount of 6-MP is from about 5 nM to about 1105 nM. In embodiments, the effective amount of 6-MP is from about 5 nM to about 1055 nM. In embodiments, the effective amount of 6-MP is from about 5 nM to about 1005 nM. In embodiments, the effective amount of 6-MP is from about 5 nM to about 955 nM. In embodiments, the effective amount of 6-MP is from about 5 nM to about 905 nM. In embodiments, the effective amount of 6-MP is from about 5 nM to about 855 nM. In embodiments, the effective amount of 6-MP is from about 5 nM to about 805 nM. In embodiments, the effective amount of 6-MP is from about 5 nM to about 755 nM. In embodiments, the effective amount of 6-MP is from about 5 nM to about 705 nM. In embodiments, the effective amount of 6-MP is from about 5 nM to about 655 nM. In embodiments, the effective amount of 6-MP is from about 5 nM to about 605 nM. In embodiments, the effective amount of 6-MP is from about 5 nM to about 555 nM. In embodiments, the effective amount of 6-MP is from about 5 nM to about 505 nM. In embodiments, the effective amount of 6-MP is from about 5 nM to about 455 nM. In embodiments, the effective amount of 6-MP is from about 5 nM to about 405 nM. In embodiments, the effective amount of 6-MP is from about 5 nM to about 355 nM. In embodiments, the effective amount of 6-MP is from about 5 nM to about 305 nM. In embodiments, the effective amount of 6-MP is from about 5 nM to about 255 nM. In embodiments, the effective amount of 6-MP is from about 5 nM to about 205 nM. In embodiments, the effective amount of 6-MP is from about 5 nM to about 155 nM. In embodiments, the effective amount of 6-MP is from about 5 nM to about 105 nM. In embodiments, the effective amount of 6-MP is from about 5 nM to about 55 nM. In embodiments, the effective amount of 6-MP is about 5 nM, 55 nM, 105 nM, 155 nM, 205 nM, 255 nM, 305 nM, 355 nM, 405 nM, 455 nM, 505 nM, 555 nM, 605 nM, 655 nM, 705 nM, 755 nM, 805 nM, 855 nM, 905 nM, 955 nM, 1005 nM, 1055 nM, 1105 nM, 1155 nM, 1205 nM, or 1255 nM. In embodiments, the effective amount of 6-MP is about 1000 nM. In embodiments, the effective amount of 6-MP is 1000 nM.

In embodiments, the amount of 6-MP effective to cause cell death in non-transduced T-cells is from about 5 nM to about 120 nM. In embodiments, the effective amount of 6-MP is from about 10 nM to about 120 nM. In embodiments, the effective amount of 6-MP is from about 15 nM to about 120 nM. In embodiments, the effective amount of 6-MP is from about 20 nM to about 120 nM. In embodiments, the effective amount of 6-MP is from about 25 nM to about 120 nM. In embodiments, the effective amount of 6-MP is from about 30 nM to about 120 nM. In embodiments, the effective amount of 6-MP is from about 35 nM to about 120 nM. In embodiments, the effective amount of 6-MP is from about 40 nM to about 120 nM. In embodiments, the effective amount of 6-MP is from about 45 nM to about 120 nM. In embodiments, the effective amount of 6-MP is from about 50 nM to about 120 nM. In embodiments, the effective amount of 6-MP is from about 55 nM to about 120 nM. In embodiments, the effective amount of 6-MP is from about 60 nM to about 120 nM. In embodiments, the effective amount of 6-MP is from about 65 nM to about 120 nM. In embodiments, the effective amount of 6-MP is from about 70 nM to about 120 nM. In embodiments, the effective amount of 6-MP is from about 75 nM to about 120 nM. In embodiments, the effective amount of 6-MP is from about 80 nM to about 120 nM. In embodiments, the effective amount of 6-MP is from about 85 nM to about 120 nM. In embodiments, the effective amount of 6-MP is from about 90 nM to about 120 nM. In embodiments, the effective amount of 6-MP is from about 95 nM to about 120 nM. In embodiments, the effective amount of 6-MP is from about 100 nM to about 120 nM. In embodiments, the effective amount of 6-MP is from about 105 nM to about 120 nM. In embodiments, the effective amount of 6-MP is from about 110 nM to about 120 nM. In embodiments, the effective amount of 6-MP is from about 115 nM to about 120 nM.

In embodiments, the amount of 6-MP effective to cause cell death in non-transduced T-cells is from about 5 nM to about 115 nM. In embodiments, the effective amount of 6-MP is from about 5 nM to about 110 nM. In embodiments, the effective amount of 6-MP is from about 5 nM to about 105 nM. In embodiments, the effective amount of 6-MP is from about 5 nM to about 100 nM. In embodiments, the effective amount of 6-MP is from about 5 nM to about 95 nM. In embodiments, the effective amount of 6-MP is from about 5 nM to about 90 nM. In embodiments, the effective amount of 6-MP is from about 5 nM to about 85 nM. In embodiments, the effective amount of 6-MP is from about 5 nM to about 80 nM. In embodiments, the effective amount of 6-MP is from about 5 nM to about 75 nM. In embodiments, the effective amount of 6-MP is from about 5 nM to about 70 nM. In embodiments, the effective amount of 6-MP is from about 5 nM to about 65 nM. In embodiments, the effective amount of 6-MP is from about 5 nM to about 60 nM. In embodiments, the effective amount of 6-MP is from about 5 nM to about 55 nM. In embodiments, the effective amount of 6-MP is from about 5 nM to about 50 nM. In embodiments, the effective amount of 6-MP is from about 5 nM to about 45 nM. In embodiments, the effective amount of 6-MP is from about 5 nM to about 40 nM. In embodiments, the effective amount of 6-MP is from about 5 nM to about 35 nM. In embodiments, the effective amount of 6-MP is from about 5 nM to about 30 nM. In embodiments, the effective amount of 6-MP is from about 5 nM to about 25 nM. In embodiments, the effective amount of 6-MP is from about 5 nM to about 20 nM. In embodiments, the effective amount of 6-MP is from about 5 nM to about 15 nM. In embodiments, the effective amount of 6-MP is from about 5 nM to about 10 nM. In embodiments, the effective amount of 6-MP is about 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, or 120 nM. In embodiments, the effective amount of 6-MP is about 10 nM. In embodiments, the effective amount of 6-MP is 10 nM. In embodiments, the effective amount of 6-MP is about 20 nM. In embodiments, the effective amount of 6-MP is 20 nM. In embodiments, the effective amount of 6-MP is about 50 nM. In embodiments, the effective amount of 6-MP is 50 nM.

In embodiments, the HPRT inhibitor is anti-HPRT shRNA. In embodiments, the anti-HPRT shRNA includes a sequence having at least 80% sequence identity to SEQ ID NO:12. In embodiments, the nucleic acid further includes a sequence encoding a Tat/Rev inhibitor. In embodiments, the Tat/Rev inhibitor is an anti-Tat/Rev shRNA. In embodiments, the anti-Tat/Rev shRNA includes a sequence having at least 80% sequence identity to SEQ ID NO:10 In embodiments, the nucleic acid further includes a sequence encoding a CCR5 inhibitor. In embodiments, the CCR5 inhibitor is an anti-CCR5 shRNA. In embodiments, the anti-CCR5 shRNA includes a sequence having at least 80% sequence identity to SEQ ID NO:9 or 11.

In embodiments, the one or more of the anti-Tat/Rev shRNA, the anti-CCR5 shRNA, or the anti-HPRT shRNA includes a wobble base.

In embodiments, the anti-HIV CAR includes: (i) an antibody region capable of binding an HIV envelope protein; and (ii) a transmembrane domain. In embodiments, the HIV envelope protein is gp120 or gp41. In embodiments, the HIV envelope protein is gp120. In embodiments, the HIV envelope protein is gp41.

In embodiments, the antibody region includes a heavy chain variable domain including the sequence of SEQ ID NO:4, and a light chain variable domain including the sequence of SEQ ID NO:5. In embodiments, the antibody region includes a heavy chain variable domain including the sequence of SEQ ID NO:6, and a light chain variable domain including the sequence of SEQ ID NO:7.

In embodiments, the population of T-cells is obtained from the subject. In embodiments, the population of T-cells is obtained from a donor who is not infected with HIV. In embodiments, the population of selected transduced T-cells are administered intravenously.

In another aspect is provided a method of treating an HIV infected subject in need thereof, the method including: contacting a plurality of T-cells including a T-cell provided herein including embodiments thereof with an amount of 6-thioguanine (6TG) effective to cause hematopoietic toxicity in T-cells that are non-transduced with the nucleic acid encoding an HPRT inhibitor thereby resulting in selection of cells transduced with said HPRT inhibitor; and administering to the subject an effective amount of the selected cells. In embodiments, the plurality of T-cells are obtained from the subject infected with HIV. In embodiments, the plurality of T-cells are not obtained from the subject infected with HIV.

### Methods of Selecting T-Cells

The T-cells provided herein including embodiments thereof, are useful for selecting transduced T-cells from a population of transduced and non-transduced T-cells. A transduced T-cell includes a nucleic acid including a sequence encoding the anti-HIV CAR and a sequence encoding a protein inhibitor (e.g. HPRT inhibitor) as provided herein including embodiments thereof. Thus, a non-transduced T-cell does not include the nucleic acid having a sequence encoding the anti-HIV CAR and a sequence encoding a protein inhibitor (e.g. HPRT inhibitor). Applicant demonstrates herein that the selection of transduced T-cells (e.g. T-cells provided herein) from a population of transduced and non-transduced T-cells results in a substantially pure population of transduced T-cells. Thus, in an aspect is provided a method of selecting for T-cells including: (i) contacting a population of T-cells with a nucleic acid, thereby forming a population of transduced T-cells and non-transduced T-cells, wherein the nucleic acid includes a sequence encoding an anti-HIV Chimeric Antigen Receptor (CAR) and a sequence encoding a HPRT inhibitor; and (ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells. In embodiments, the population of transduced and non-transduced T-cells is contacted with an effective amount of 6TG. In embodiments, the population of transduced and non-transduced T-cells is contacted with an effective amount of 6-MP.

As described above, the population of transduced and non-transduced T-cells include T-cells comprising the nucleic acid provided herein (e.g. transduced T-cells) and T-cells that do not comprise the nucleic acid provided herein (e.g. non-transduced T-cells). In embodiments, the population of transduced and non-transduced T-cells is due to incomplete transduction. In embodiments, the population of transduced and non-transduced T-cells is due to transduction efficiencies below 100%. Thus, in embodiments, the contacting the population of T-cells with the nucleic acid of step i) includes delivering the nucleic acid into a subset of the T-cells. In embodiments, the nucleic acid is delivered by a virus like particle or a virus. In embodiments, the nucleic acid is part of a lentiviral vector and is delivered by transduction into a T-cell. In embodiments, the population of T-cells of step i) are contacted with a virus (e.g. lentivirus etc.) including the nucleic acid provided herein including embodiments thereof at a multiplicity of infection (MOI) of 0.1 to 10. In embodiments, the population of T-cells are contacted with the virus at a MOI of 0.5 to 10. In embodiments, the population of T-cells are contacted with the virus at a MOI of 0.5 to 10. In embodiments, the population of T-cells are contacted with the virus at a MOI of 1 to 10. In embodiments, the population of T-cells are contacted with the virus at a MOI of 1.5 to 10. In embodiments, the population of T-cells are contacted with the virus at a MOI of 2 to 10. In embodiments, the population of T-cells are contacted with the virus at a MOI of 2.5 to 10. In embodiments, the population of T-cells are contacted with the virus at a MOI of 3 to 10. In embodiments, the population of T-cells are contacted with the virus at a MOI of 3.5 to 10. In embodiments, the population of T-cells are contacted with the virus at a MOI of 4 to 10. In embodiments, the population of T-cells are contacted with the virus at a MOI of 4.5 to 10. In embodiments, the population of T-cells are contacted with the virus at a MOI of 5 to 10. In embodiments, the population of T-cells are contacted with the virus at a MOI of 5.5 to 10. In embodiments, the population of T-cells are contacted with the virus at a MOI of 6 to 10. In embodiments, the population of T-cells are contacted with the virus at a MOI of 6.5 to 10. In embodiments, the population of T-cells are contacted with the virus at a MOI of 7 to 10. In embodiments, the population of T-cells are contacted with the virus at a MOI of 7.5 to 10. In embodiments, the population of T-cells are contacted with the virus at a MOI of 8 to 10. In embodiments, the population of T-cells are contacted with the virus at a MOI of 8.5 to 10. In embodiments, the population of T-cells are contacted with the virus at a MOI of 9 to 10. In embodiments, the population of T-cells are contacted with the virus at a MOI of 9.5 to 10.

In embodiments, the population of T-cells are contacted with the virus at a MOI of 0.1 to 9.5. In embodiments, the population of T-cells are contacted with the virus at a MOI of 0.1 to 9. In embodiments, the population of T-cells are contacted with the virus at a MOI of 0.1 to 8.5. In embodiments, the population of T-cells are contacted with the virus at a MOI of 0.1 to 8. In embodiments, the population of T-cells are contacted with the virus at a MOI of 0.1 to 7.5. In embodiments, the population of T-cells are contacted with the virus at a MOI of 0.1 to 7. In embodiments, the population of T-cells are contacted with the virus at a MOI of 0.1 to 6.5. In embodiments, the population of T-cells are contacted with the virus at a MOI of 0.1 to 6. In embodiments, the population of T-cells are contacted with the virus at a MOI of 0.1 to 5.5. In embodiments, the population of T-cells are contacted with the virus at a MOI of 0.1 to 5. In embodiments, the population of T-cells are contacted with the virus at a MOI of 0.1 to 4.5. In embodiments, the population of T-cells are contacted with the virus at a MOI of 0.1 to 4. In embodiments, the population of T-cells are contacted with the virus at a MOI of 0.1 to 3.5. In embodiments, the population of T-cells are contacted with the virus at a MOI of 0.1 to 3. In embodiments, the population of T-cells are contacted with the virus at a MOI of 0.1 to 2.5. In embodiments, the population of T-cells are contacted with the virus at a MOI of 0.1 to 2. In embodiments, the population of T-cells are contacted with the virus at a MOI of 0.1 to 1.5. In embodiments, the population of T-cells are contacted with the virus at a MOI of 0.1 to 1. In embodiments, the population of T-cells are contacted with the virus at a MOI of 0.1 to 0.5. In embodiments, the population of T-cells are contacted with the virus at a MOI of 0.1, 0.5, 1, 1.5, 2, 2.5, 3, 3.5, 4, 4.5, 5, 5.5, 6, 6.5 7, 7.5, 8, 8.5, 9, 9.5 or 10. In embodiments, the population of T-cells are contacted with the virus at a MOI of about 2. In embodiments, the population of T-cells are contacted with the virus at a MOI of 2.

In embodiments, the selected T-cells or population of selected transduced T-cells are a substantially pure population of the T-cells provided herein including embodiments thereof. As used herein, “substantially pure” population of transduced T-cells (e.g. population of selected transduced T-cells) or T-cells refers to a population of cells wherein at least about 50% to 100% of cells in the population are the T-cells provided herein including embodiments thereof (e.g. T-cells including a nucleic acid including a sequence encoding an anti-HIV CAR and a sequence encoding one or more protein inhibitor (e.g. HPRT inhibitor)). In embodiments, a substantially pure population includes about 50% to 100% of the T-cells provided herein including embodiments thereof (e.g. T-cells including a nucleic acid including a sequence encoding an anti-HIV CAR and a sequence encoding a protein inhibitor (e.g. an HPRT inhibitor)). In embodiments, a substantially pure population includes about 55% to 100% of T-cells provided herein including embodiments thereof. In embodiments, a substantially pure population includes about 60% to 100% of T-cells provided herein including embodiments thereof. In embodiments, a substantially pure population includes about 65% to 100% of T-cells provided herein including embodiments thereof. In embodiments, a substantially pure population includes about 70% to 100% of T-cells provided herein including embodiments thereof. In embodiments, a substantially pure population includes about 75% to 100% of T-cells provided herein including embodiments thereof. In embodiments, a substantially pure population includes about 80% to 100% of T-cells provided herein including embodiments thereof. In embodiments, a substantially pure population includes about 85% to 100% of T-cells provided herein including embodiments thereof. In embodiments, a substantially pure population includes about 90% to 100% of T-cells provided herein including embodiments thereof. In embodiments, a substantially pure population includes about 95% to 100% of T-cells provided herein including embodiments thereof.

In embodiments, a substantially pure population includes about 50% to 95% of T-cells provided herein including embodiments thereof. In embodiments, a substantially pure population includes about 50% to 90% of T-cells provided herein including embodiments thereof. In embodiments, a substantially pure population includes about 50% to 85% of T-cells provided herein including embodiments thereof. In embodiments, a substantially pure population includes about 50% to 80% of T-cells provided herein including embodiments thereof. In embodiments, a substantially pure population includes about 50% to 75% of T-cells provided herein including embodiments thereof. In embodiments, a substantially pure population includes about 50% to 70% of T-cells provided herein including embodiments thereof. In embodiments, a substantially pure population includes about 50% to 65% of T-cells provided herein including embodiments thereof. In embodiments, a substantially pure population includes about 50% to 60% of T-cells provided herein including embodiments thereof. In embodiments, a substantially pure population includes about 50% to 55% of T-cells provided herein including embodiments thereof. In embodiments, a substantially pure population includes about 50% to 95% of T-cells provided herein including embodiments thereof. In embodiments, a substantially pure population includes about 50%, 55%, 60%, 65%, 70%, 75%, 80%, 85%, 90%, 95%, or 100% T-cells provided herein including embodiments thereof.

In embodiments, a substantially pure population includes about 70% to 100% of the T-cells provided herein including embodiments thereof (e.g. T-cells including a nucleic acid including a sequence encoding an anti-HIV CAR and a sequence encoding a protein inhibitor (e.g. an HPRT inhibitor)). In embodiments, a substantially pure population includes about 73% to 100% of the T-cells provided herein including embodiments thereof. In embodiments, a substantially pure population includes about 76% to 100% of the T-cells provided herein including embodiments thereof. In embodiments, a substantially pure population includes about 79% to 100% of the T-cells provided herein including embodiments thereof. In embodiments, a substantially pure population includes about 82% to 100% of the T-cells provided herein including embodiments thereof. In embodiments, a substantially pure population includes about 85% to 100% of the T-cells provided herein including embodiments thereof. In embodiments, a substantially pure population includes about 88% to 100% of the T-cells provided herein including embodiments thereof. In embodiments, a substantially pure population includes about 91% to 100% of the T-cells provided herein including embodiments thereof. In embodiments, a substantially pure population includes about 94% to 100% of the T-cells provided herein including embodiments thereof. In embodiments, a substantially pure population includes about 97% to 100% of the T-cells provided herein including embodiments thereof.

In embodiments, a substantially pure population includes about 70% to 97% of the T-cells provided herein including embodiments thereof. In embodiments, a substantially pure population includes about 70% to 94% of the T-cells provided herein including embodiments thereof. In embodiments, a substantially pure population includes about 70% to 91% of the T-cells provided herein including embodiments thereof. In embodiments, a substantially pure population includes about 70% to 88% of the T-cells provided herein including embodiments thereof. In embodiments, a substantially pure population includes about 70% to 85% of the T-cells provided herein including embodiments thereof. In embodiments, a substantially pure population includes about 70% to 82% of the T-cells provided herein including embodiments thereof. In embodiments, a substantially pure population includes about 70% to 79% of the T-cells provided herein including embodiments thereof. In embodiments, a substantially pure population includes about 70% to 76% of the T-cells provided herein including embodiments thereof. In embodiments, a substantially pure population includes about 70% to 73% of the T-cells provided herein including embodiments thereof. In embodiments, a substantially pure population includes about 70%, 73%, 76%, 79%, 82%, 85%, 88%, 91%, 94%, 97%, or 100% T-cells provided herein including embodiments thereof. In embodiments, a substantially pure population includes at least 80% of the T-cells provided herein including embodiments thereof. In embodiments, a substantially pure population includes about 80% of the T-cells provided herein including embodiments thereof. In embodiments, a substantially pure population includes 80% of the T-cells provided herein including embodiments thereof.

The population of T-cells including transduced (e.g. T-cells including the nucleic acid including a sequence encoding an anti-HIV CAR and a sequence encoding a protein inhibitor (e.g. HPRT inhibitor)) and non-transduced T-cells may be contacted with 6TG or 6-MP, wherein the contacting occurs by addition of 6TG or 6-MP into a cell culture including the population of T-cells. In instances, an effective amount of 6TG or 6-MP may be added to the cell culture including transduced and non-transduced T-cells a plurality of times to cause cell death in non-transduced cells. Thus, in embodiments, the population of T-cells is contacted with the effective amount of 6TG or 6-MP a plurality of times.

In embodiments, the population of T-cells is contacted with the effective amount of 6TG a plurality of times effective to cause cell death in the non-transduced T-cells. In embodiments, the population of T-cells is contacted with the effective amount of 6TG two times. In embodiments, the population of T-cells is contacted with the effective amount of 6TG three times. In embodiments, the population of T-cells is contacted with the effective amount of 6TG four times. In embodiments, the population of T-cells is contacted with the effective amount of 6TG five times. In embodiments, the population of T-cells is contacted with the effective amount of 6TG six times. In embodiments, the population of T-cells is contacted with the effective amount of 6TG seven times. In embodiments, the population of T-cells is contacted with the effective amount of 6TG eight times.

In embodiments, the population of T-cells is contacted with the effective amount of 6TG once every 12 hours. In embodiments, the population of T-cells is contacted with the effective amount of 6TG once every 24 hours. In embodiments, the population of T-cells is contacted with the effective amount of 6TG once every 36 hours. In embodiments, the population of T-cells is contacted with the effective amount of 6TG once every 48 hours.

In embodiments, the population of T-cells is contacted with the effective amount of 6-MP a plurality of times effective to cause cell death in the non-transduced T-cells. In embodiments, the population of T-cells is contacted with the effective amount of 6-MP two times. In embodiments, the population of T-cells is contacted with the effective amount of 6-MP three times. In embodiments, the population of T-cells is contacted with the effective amount of 6-MP four times. In embodiments, the population of T-cells is contacted with the effective amount of 6-MP five times. In embodiments, the population of T-cells is contacted with the effective amount of 6-MP six times. In embodiments, the population of T-cells is contacted with the effective amount of 6-MP seven times. In embodiments, the population of T-cells is contacted with the effective amount of 6-MP eight times.

In embodiments, the population of T-cells is contacted with the effective amount of 6-MP once every 12 hours. In embodiments, the population of T-cells is contacted with the effective amount of 6-MP once every 24 hours. In embodiments, the population of T-cells is contacted with the effective amount of 6-MP once every 36 hours. In embodiments, the population of T-cells is contacted with the effective amount of 6-MP once every 48 hours.

In embodiments, the amount of 6TG effective to cause cell death in the non-transduced T-cells (e.g. effective amount of 6TG) is from about 5 nM to about 1255 nM. In embodiments, the effective amount of 6TG is from about 55 nM to about 1255 nM In embodiments, the effective amount of 6TG is from about 55 nM to about 1255 nM. In embodiments, the effective amount of 6TG is from about 105 nM to about 1255 nM. In embodiments, the effective amount of 6TG is from about 155 nM to about 1255 nM. In embodiments, the effective amount of 6TG is from about 205 nM to about 1255 nM. In embodiments, the effective amount of 6TG is from about 255 nM to about 1255 nM. In embodiments, the effective amount of 6TG is from about 305 nM to about 1255 nM. In embodiments, the effective amount of 6TG is from about 355 nM to about 1255 nM. In embodiments, the effective amount of 6TG is from about 405 nM to about 1255 nM. In embodiments, the effective amount of 6TG is from about 455 nM to about 1255 nM. In embodiments, the effective amount of 6TG is from about 505 nM to about 1255 nM. In embodiments, the effective amount of 6TG is from about 555 nM to about 1255 nM. In embodiments, the effective amount of 6TG is from about 605 nM to about 1255 nM. In embodiments, the effective amount of 6TG is from about 655 nM to about 1255 nM. In embodiments, the effective amount of 6TG is from about 705 nM to about 1255 nM. In embodiments, the effective amount of 6TG is from about 755 nM to about 1255 nM. In embodiments, the effective amount of 6TG is from about 805 nM to about 1255 nM. In embodiments, the effective amount of 6TG is from about 855 nM to about 1255 nM. In embodiments, the effective amount of 6TG is from about 905 nM to about 1255 nM. In embodiments, the effective amount of 6TG is from about 955 nM to about 1255 nM. In embodiments, the effective amount of 6TG is from about 1005 nM to about 1255 nM. In embodiments, the effective amount of 6TG is from about 1055 nM to about 1255 nM. In embodiments, the effective amount of 6TG is from about 1105 nM to about 1255 nM. In embodiments, the effective amount of 6TG is from about 1155 nM to about 1255 nM. In embodiments, the effective amount of 6TG is from about 1205 nM to about 1255 nM. In embodiments, the effective amount of 6TG is from about 1255 nM to about 1255 nM.

In embodiments, the amount of 6TG effective to cause cell death in the non-transduced T-cells (e.g. effective amount of 6TG) is from about 5 nM to about 1205 nM. In embodiments, the effective amount of 6TG is from about 5 nM to about 1055 nM. In embodiments, the effective amount of 6TG is from about 5 nM to about 1105 nM. In embodiments, the effective amount of 6TG is from about 5 nM to about 1055 nM. In embodiments, the effective amount of 6TG is from about 5 nM to about 1005 nM. In embodiments, the effective amount of 6TG is from about 5 nM to about 955 nM. In embodiments, the effective amount of 6TG is from about 5 nM to about 905 nM. In embodiments, the effective amount of 6TG is from about 5 nM to about 855 nM. In embodiments, the effective amount of 6TG is from about 5 nM to about 805 nM. In embodiments, the effective amount of 6TG is from about 5 nM to about 755 nM. In embodiments, the effective amount of 6TG is from about 5 nM to about 705 nM. In embodiments, the effective amount of 6TG is from about 5 nM to about 655 nM. In embodiments, the effective amount of 6TG is from about 5 nM to about 605 nM. In embodiments, the effective amount of 6TG is from about 5 nM to about 555 nM. In embodiments, the effective amount of 6TG is from about 5 nM to about 505 nM. In embodiments, the effective amount of 6TG is from about 5 nM to about 455 nM. In embodiments, the effective amount of 6TG is from about 5 nM to about 405 nM. In embodiments, the effective amount of 6TG is from about 5 nM to about 355 nM. In embodiments, the effective amount of 6TG is from about 5 nM to about 305 nM. In embodiments, the effective amount of 6TG is from about 5 nM to about 255 nM. In embodiments, the effective amount of 6TG is from about 5 nM to about 205 nM. In embodiments, the effective amount of 6TG is from about 5 nM to about 155 nM. In embodiments, the effective amount of 6TG is from about 5 nM to about 105 nM. In embodiments, the effective amount of 6TG is from about 5 nM to about 55 nM. In embodiments, the effective amount of 6TG is about 5 nM, 55 nM, 105 nM, 155 nM, 205 nM, 255 nM, 305 nM, 355 nM, 405 nM, 455 nM, 505 nM, 555 nM, 605 nM, 655 nM, 705 nM, 755 nM, 805 nM, 855 nM, 905 nM, 955 nM, 1005 nM, 1055 nM, 1105 nM, 1155 nM, 1205 nM, or 1255 nM. In embodiments, the effective amount of 6TG is about 1000 nM. In embodiments, the effective amount of 6TG is 1000 nM.

In embodiments, the amount of 6TG effective to cause cell death in the non-transduced T-cells (e.g. effective amount of 6TG) is from about 5 nM to about 120 nM. In embodiments, the effective amount of 6TG is from about 10 nM to about 120 nM. In embodiments, the effective amount of 6TG is from about 15 nM to about 120 nM. In embodiments, the effective amount of 6TG is from about 20 nM to about 120 nM. In embodiments, the effective amount of 6TG is from about 25 nM to about 120 nM. In embodiments, the effective amount of 6TG is from about 30 nM to about 120 nM. In embodiments, the effective amount of 6TG is from about 35 nM to about 120 nM. In embodiments, the effective amount of 6TG is from about 40 nM to about 120 nM. In embodiments, the effective amount of 6TG is from about 45 nM to about 120 nM. In embodiments, the effective amount of 6TG is from about 50 nM to about 120 nM. In embodiments, the effective amount of 6TG is from about 55 nM to about 120 nM. In embodiments, the effective amount of 6TG is from about 60 nM to about 120 nM. In embodiments, the effective amount of 6TG is from about 65 nM to about 120 nM. In embodiments, the effective amount of 6TG is from about 70 nM to about 120 nM. In embodiments, the effective amount of 6TG is from about 75 nM to about 120 nM. In embodiments, the effective amount of 6TG is from about 80 nM to about 120 nM. In embodiments, the effective amount of 6TG is from about 85 nM to about 120 nM. In embodiments, the effective amount of 6TG is from about 90 nM to about 120 nM. In embodiments, the effective amount of 6TG is from about 95 nM to about 120 nM. In embodiments, the effective amount of 6TG is from about 100 nM to about 120 nM. In embodiments, the effective amount of 6TG is from about 105 nM to about 120 nM. In embodiments, the effective amount of 6TG is from about 110 nM to about 120 nM. In embodiments, the effective amount of 6TG is from about 115 nM to about 120 nM.

In embodiments, the amount of 6TG effective to cause cell death in the non-transduced T-cells (e.g. effective amount of 6TG) is from about 5 nM to about 115 nM. In embodiments, the effective amount of 6TG is from about 5 nM to about 110 nM. In embodiments, the effective amount of 6TG is from about 5 nM to about 105 nM. In embodiments, the effective amount of 6TG is from about 5 nM to about 100 nM. In embodiments, the effective amount of 6TG is from about 5 nM to about 95 nM. In embodiments, the effective amount of 6TG is from about 5 nM to about 90 nM. In embodiments, the effective amount of 6TG is from about 5 nM to about 85 nM. In embodiments, the effective amount of 6TG is from about 5 nM to about 80 nM. In embodiments, the effective amount of 6TG is from about 5 nM to about 75 nM. In embodiments, the effective amount of 6TG is from about 5 nM to about 70 nM. In embodiments, the effective amount of 6TG is from about 5 nM to about 65 nM. In embodiments, the effective amount of 6TG is from about 5 nM to about 60 nM. In embodiments, the effective amount of 6TG is from about 5 nM to about 55 nM. In embodiments, the effective amount of 6TG is from about 5 nM to about 50 nM. In embodiments, the effective amount of 6TG is from about 5 nM to about 45 nM. In embodiments, the effective amount of 6TG is from about 5 nM to about 40 nM. In embodiments, the effective amount of 6TG is from about 5 nM to about 35 nM. In embodiments, the effective amount of 6TG is from about 5 nM to about 30 nM. In embodiments, the effective amount of 6TG is from about 5 nM to about 25 nM. In embodiments, the effective amount of 6TG is from about 5 nM to about 20 nM. In embodiments, the effective amount of 6TG is from about 5 nM to about 15 nM. In embodiments, the effective amount of 6TG is from about 5 nM to about 10 nM. In embodiments, the effective amount of 6TG is about 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, or 120 nM. In embodiments, the effective amount of 6TG is about 10 nM. In embodiments, the effective amount of 6TG is 10 nM. In embodiments, the effective amount of 6TG is about 20 nM. In embodiments, the effective amount of 6TG is 20 nM. In embodiments, the effective amount of 6TG is about 50 nM. In embodiments, the effective amount of 6TG is 50 nM.

In embodiments, the amount of 6-MP effective to cause cell death in the non-transduced T-cells (e.g. effective amount of 6-MP) is from about 5 nM to about 1255 nM. In embodiments, the effective amount of 6-MP is from about 55 nM to about 1255 nM In embodiments, the effective amount of 6-MP is from about 55 nM to about 1255 nM. In embodiments, the effective amount of 6-MP is from about 105 nM to about 1255 nM. In embodiments, the effective amount of 6-MP is from about 155 nM to about 1255 nM. In embodiments, the effective amount of 6-MP is from about 205 nM to about 1255 nM. In embodiments, the effective amount of 6-MP is from about 255 nM to about 1255 nM. In embodiments, the effective amount of 6-MP is from about 305 nM to about 1255 nM. In embodiments, the effective amount of 6-MP is from about 355 nM to about 1255 nM. In embodiments, the effective amount of 6-MP is from about 405 nM to about 1255 nM. In embodiments, the effective amount of 6-MP is from about 455 nM to about 1255 nM. In embodiments, the effective amount of 6-MP is from about 505 nM to about 1255 nM. In embodiments, the effective amount of 6-MP is from about 555 nM to about 1255 nM. In embodiments, the effective amount of 6-MP is from about 605 nM to about 1255 nM. In embodiments, the effective amount of 6-MP is from about 655 nM to about 1255 nM. In embodiments, the effective amount of 6-MP is from about 705 nM to about 1255 nM. In embodiments, the effective amount of 6-MP is from about 755 nM to about 1255 nM. In embodiments, the effective amount of 6-MP is from about 805 nM to about 1255 nM. In embodiments, the effective amount of 6-MP is from about 855 nM to about 1255 nM. In embodiments, the effective amount of 6-MP is from about 905 nM to about 1255 nM. In embodiments, the effective amount of 6-MP is from about 955 nM to about 1255 nM. In embodiments, the effective amount of 6-MP is from about 1005 nM to about 1255 nM. In embodiments, the effective amount of 6-MP is from about 1055 nM to about 1255 nM. In embodiments, the effective amount of 6-MP is from about 1105 nM to about 1255 nM. In embodiments, the effective amount of 6-MP is from about 1155 nM to about 1255 nM. In embodiments, the effective amount of 6-MP is from about 1205 nM to about 1255 nM.

In embodiments, the amount of 6-MP effective to cause cell death in the non-transduced T-cells (e.g. effective amount of 6-MP) is from about 5 nM to about 1205 nM. In embodiments, the effective amount of 6-MP is from about 5 nM to about 1055 nM. In embodiments, the effective amount of 6-MP is from about 5 nM to about 1105 nM. In embodiments, the effective amount of 6-MP is from about 5 nM to about 1055 nM. In embodiments, the effective amount of 6-MP is from about 5 nM to about 1005 nM. In embodiments, the effective amount of 6-MP is from about 5 nM to about 955 nM. In embodiments, the effective amount of 6-MP is from about 5 nM to about 905 nM. In embodiments, the effective amount of 6-MP is from about 5 nM to about 855 nM. In embodiments, the effective amount of 6-MP is from about 5 nM to about 805 nM. In embodiments, the effective amount of 6-MP is from about 5 nM to about 755 nM. In embodiments, the effective amount of 6-MP is from about 5 nM to about 705 nM. In embodiments, the effective amount of 6-MP is from about 5 nM to about 655 nM. In embodiments, the effective amount of 6-MP is from about 5 nM to about 605 nM. In embodiments, the effective amount of 6-MP is from about 5 nM to about 555 nM. In embodiments, the effective amount of 6-MP is from about 5 nM to about 505 nM. In embodiments, the effective amount of 6-MP is from about 5 nM to about 455 nM. In embodiments, the effective amount of 6-MP is from about 5 nM to about 405 nM. In embodiments, the effective amount of 6-MP is from about 5 nM to about 355 nM. In embodiments, the effective amount of 6-MP is from about 5 nM to about 305 nM. In embodiments, the effective amount of 6-MP is from about 5 nM to about 255 nM. In embodiments, the effective amount of 6-MP is from about 5 nM to about 205 nM. In embodiments, the effective amount of 6-MP is from about 5 nM to about 155 nM. In embodiments, the effective amount of 6-MP is from about 5 nM to about 105 nM. In embodiments, the effective amount of 6-MP is from about 5 nM to about 55 nM. In embodiments, the effective amount of 6-MP is about 5 nM, 55 nM, 105 nM, 155 nM, 205 nM, 255 nM, 305 nM, 355 nM, 405 nM, 455 nM, 505 nM, 555 nM, 605 nM, 655 nM, 705 nM, 755 nM, 805 nM, 855 nM, 905 nM, 955 nM, 1005 nM, 1055 nM, 1105 nM, 1155 nM, 1205 nM, or 1255 nM. In embodiments, the effective amount of 6-MP is about 1000 nM. In embodiments, the effective amount of 6-MP is 1000 nM.

In embodiments, the amount of 6-MP effective to cause cell death in the non-transduced T-cells (e.g. effective amount of 6-MP) is from about 5 nM to about 120 nM. In embodiments, the effective amount of 6-MP is from about 10 nM to about 120 nM. In embodiments, the effective amount of 6-MP is from about 15 nM to about 120 nM. In embodiments, the effective amount of 6-MP is from about 20 nM to about 120 nM. In embodiments, the effective amount of 6-MP is from about 25 nM to about 120 nM. In embodiments, the effective amount of 6-MP is from about 30 nM to about 120 nM. In embodiments, the effective amount of 6-MP is from about 35 nM to about 120 nM. In embodiments, the effective amount of 6-MP is from about 40 nM to about 120 nM. In embodiments, the effective amount of 6-MP is from about 45 nM to about 120 nM. In embodiments, the effective amount of 6-MP is from about 50 nM to about 120 nM. In embodiments, the effective amount of 6-MP is from about 55 nM to about 120 nM. In embodiments, the effective amount of 6-MP is from about 60 nM to about 120 nM. In embodiments, the effective amount of 6-MP is from about 65 nM to about 120 nM. In embodiments, the effective amount of 6-MP is from about 70 nM to about 120 nM. In embodiments, the effective amount of 6-MP is from about 75 nM to about 120 nM. In embodiments, the effective amount of 6-MP is from about 80 nM to about 120 nM. In embodiments, the effective amount of 6-MP is from about 85 nM to about 120 nM. In embodiments, the effective amount of 6-MP is from about 90 nM to about 120 nM. In embodiments, the effective amount of 6-MP is from about 95 nM to about 120 nM. In embodiments, the effective amount of 6-MP is from about 100 nM to about 120 nM. In embodiments, the effective amount of 6-MP is from about 105 nM to about 120 nM. In embodiments, the effective amount of 6-MP is from about 110 nM to about 120 nM. In embodiments, the effective amount of 6-MP is from about 115 nM to about 120 nM.

In embodiments, the amount of 6-MP effective to cause cell death in the non-transduced T-cells (e.g. effective amount of 6-MP) is from about 5 nM to about 115 nM. In embodiments, the effective amount of 6-MP is from about 5 nM to about 110 nM. In embodiments, the effective amount of 6-MP is from about 5 nM to about 105 nM. In embodiments, the effective amount of 6-MP is from about 5 nM to about 100 nM. In embodiments, the effective amount of 6-MP is from about 5 nM to about 95 nM. In embodiments, the effective amount of 6-MP is from about 5 nM to about 90 nM. In embodiments, the effective amount of 6-MP is from about 5 nM to about 85 nM. In embodiments, the effective amount of 6-MP is from about 5 nM to about 80 nM. In embodiments, the effective amount of 6-MP is from about 5 nM to about 75 nM. In embodiments, the effective amount of 6-MP is from about 5 nM to about 70 nM. In embodiments, the effective amount of 6-MP is from about 5 nM to about 65 nM. In embodiments, the effective amount of 6-MP is from about 5 nM to about 60 nM. In embodiments, the effective amount of 6-MP is from about 5 nM to about 55 nM. In embodiments, the effective amount of 6-MP is from about 5 nM to about 50 nM. In embodiments, the effective amount of 6-MP is from about 5 nM to about 45 nM. In embodiments, the effective amount of 6-MP is from about 5 nM to about 40 nM. In embodiments, the effective amount of 6-MP is from about 5 nM to about 35 nM. In embodiments, the effective amount of 6-MP is from about 5 nM to about 30 nM. In embodiments, the effective amount of 6-MP is from about 5 nM to about 25 nM. In embodiments, the effective amount of 6-MP is from about 5 nM to about 20 nM. In embodiments, the effective amount of 6-MP is from about 5 nM to about 15 nM. In embodiments, the effective amount of 6-MP is from about 5 nM to about 10 nM. In embodiments, the effective amount of 6-MP is about 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 105 nM, 110 nM, 115 nM, or 120 nM. In embodiments, the effective amount of 6-MP is about 10 nM. In embodiments, the effective amount of 6-MP is 10 nM. In embodiments, the effective amount of 6-MP is about 20 nM. In embodiments, the effective amount of 6-MP is 20 nM. In embodiments, the effective amount of 6-MP is about 50 nM. In embodiments, the effective amount of 6-MP is 50 nM.

In embodiments, the effective amount of 6TG or 6-MP can be contacted with the population of T-cells in the presence of one or more proteins (e.g. cytokines (e.g. interleukin-15, interleukin-2, etc.)) known to facilitate T-cell expansion or activation. In embodiments, the protein is IL-2, IL-7, IL-15, IL-21, CD3, or CD28. In embodiments, the protein is IL-2. In embodiments, the protein is IL-7. In embodiments, the protein is IL-15. In embodiments, the protein is IL-21. In embodiments, the protein is CD3. In embodiments, the protein is CD28. In embodiments, 6TG and the one or more proteins (e.g. IL-2, IL-15, etc.) are contacted with the population of T-cells simultaneously. In embodiments, 6TG and the one or more proteins (e.g. IL-2, IL-15, etc.) are contacted with the population of T-cells sequentially. In embodiments, 6-MP and the one or more proteins (e.g. IL-2, IL-15, etc.) are contacted with the population of T-cells simultaneously. In embodiments, 6-MP and the one or more proteins (e.g. IL-2, IL-15, etc.) are contacted with the population of T-cells sequentially.

In embodiments, the HPRT inhibitor is anti-HPRT shRNA. In embodiments, the anti-HPRT shRNA includes a sequence having at least 80% sequence identity to SEQ ID NO:12. In embodiments, the nucleic acid further includes a sequence encoding a Tat/Rev inhibitor. In embodiments, the Tat/Rev inhibitor is an anti-Tat/Rev shRNA. In embodiments, the anti-Tat/Rev shRNA includes a sequence having at least 80% sequence identity to SEQ ID NO:10 In embodiments, the nucleic acid further includes a sequence encoding a CCR5 inhibitor. In embodiments, the CCR5 inhibitor is an anti-CCR5 shRNA. In embodiments, the anti-CCR5 shRNA includes a sequence having at least 80% sequence identity to SEQ ID NO:9 or 11.

In embodiments, the one or more of the anti-Tat/Rev shRNA, the anti-CCR5 shRNA, or the anti-HPRT shRNA includes a wobble base.

In embodiments, the anti-HIV CAR includes: (i) an antibody region capable of binding an HIV envelope protein; and (ii) a transmembrane domain. In embodiments, the HIV envelope protein is gp120 or gp41. In embodiments, the HIV envelope protein is gp120. In embodiments, the HIV envelope protein is gp41.

In embodiments, the antibody region includes a heavy chain variable domain including the sequence of SEQ ID NO:4, and a light chain variable domain including the sequence of SEQ ID NO:5. In embodiments, the antibody region includes a heavy chain variable domain including the sequence of SEQ ID NO:6, and a light chain variable domain including the sequence of SEQ ID NO:7.

### P Embodiments

Embodiment P1. A T-cell comprising: a nucleic acid encoding an anti-HIV Chimeric Antigen Receptor (CAR); and a nucleic acid encoding a Tat/Rev inhibitor.

Embodiment P2. The T-cell of embodiment P1, wherein said nucleic acid encoding said Tat/Rev inhibitor is an anti-Tat/Rev shRNA.

Embodiment P3. A T-cell comprising: a nucleic acid encoding an anti-HIV Chimeric Antigen Receptor (CAR); and a nucleic acid encoding a CCR5 inhibitor.

Embodiment P4. The T-cell of embodiment P3, wherein said nucleic acid encoding said CCR5 inhibitor is an anti-CCR5 shRNA.

Embodiment P5. The T-cell of embodiment P3 or P4, further comprising a nucleic acid encoding a Tat/Rev inhibitor.

Embodiment P6. The T-cell of embodiment P5, wherein said nucleic acid encoding said Tat/Rev inhibitor is an anti-Tat/Rev shRNA.

Embodiment P7. The T-cell of any of embodiments P1-P6, further comprising a nucleic acid encoding a hypoxanthine phosphoribosyl transferase (HPRT) inhibitor.

Embodiment P8. The T-cell of embodiment P7, wherein said HPRT inhibitor is an anti-HPRT shRNA.

Embodiment P9. A T-cell comprising: a nucleic acid encoding a Chimeric Antigen Receptor (CAR); and a nucleic acid encoding an HPRT inhibitor.

Embodiment P10. The T-cell of embodiment P9, wherein said HPRT inhibitor is an anti-HPRT shRNA.

Embodiment P11. The T-cell of any one of embodiments P1-P10, wherein said CAR comprises: (i) an antibody region comprising a heavy chain variable domain and a light chain variable domain, wherein said heavy chain variable domain comprises the sequence of SEQ ID NO:4, and wherein said light chain variable domain comprises the sequence of SEQ ID NO: 5; and (ii) a transmembrane domain

Embodiment P12. The T-cell of any one of embodiments P1-P10, wherein said CAR comprises (i) an antibody region comprising a heavy chain variable domain and a light chain variable domain, wherein said heavy chain variable domain comprises the sequence of SEQ ID NO:6, and wherein said light chain variable domain comprises the sequence of SEQ ID NO: 7; and (ii) a transmembrane domain.

Embodiment P13. A pharmaceutical composition comprising the T-cell of any of embodiments P1-P12 and a pharmaceutically-acceptable excipient.

Embodiment P14. A method of treating an HIV infected subject in need thereof, said method comprising administering to said subject an effective amount of a plurality of T-cells comprising the T-cell of any one of embodiments P1-P12.

Embodiment P15. The method of embodiment P14, wherein said plurality of T-cells are obtained from the subject infected with HIV.

Embodiment P16. The method of embodiment P14, wherein said plurality of T-cells are not obtained from the subject infected with HIV.

Embodiment P17. A method of treating an HIV infected subject in need thereof, said method comprising: contacting a plurality of T-cells comprising the T-cell of any one of embodiments P7-P10 with an amount of 6-thioguanine (6TG) effective to cause hematopoietic toxicity in T-cells that are non-transduced with said nucleic acid encoding an HPRT inhibitor thereby resulting in selection of cells transduced with said HPRT inhibitor; and administering to said subject an effective amount of said selected cells.

Embodiment P18. The method of embodiment P17, wherein said T-cell is obtained from the subject infected with HIV.

Embodiment P19. The method of embodiment P17, wherein said T-cell is not obtained from the subject infected with HIV.

Embodiment P20. A method of selecting for CAR T-cells from a population of transduced and non-transduced T-cells, said method comprising: transducing a population of T-cells with a nucleic acid encoding a HPRT inhibitor and a CAR, thereby forming a population of transduced and non-transduced T-cells; and contacting said population of transduced and non-transduced T-cells with an amount of 6-thioguanine (6TG) effective to cause hematopoietic toxicity in non-transduced T-cells, thereby resulting in selection of CAR T-cells.

Embodiment P21. The method of embodiment P20, wherein said HPRT inhibitor is anti-HPRT shRNA.

Embodiment P22. The method of embodiment P20 or P21, wherein said CAR is an anti-HIV CAR.

Embodiment P23. The method of any one of embodiments P20-P22, wherein said CAR comprises: (i) an antibody region comprising a heavy chain variable domain and a light chain variable domain, wherein said heavy chain variable domain comprises the sequence of SEQ ID NO:4, and wherein said light chain variable domain comprises the sequence of SEQ ID NO: 5; and (ii) a transmembrane domain

Embodiment P24. The T-cell of any one of embodiments P20-P22, wherein said CAR comprises: (i) an antibody region comprising a heavy chain variable domain and a light chain variable domain, wherein said heavy chain variable domain comprises the sequence of SEQ ID NO:6, and wherein said light chain variable domain comprises the sequence of SEQ ID NO: 7; and (ii) a transmembrane domain

Embodiment P25. The method of any one of embodiments P20-P24, wherein said nucleic acid further encodes a Tat/Rev inhibitor.

Embodiment P26. The method of embodiment P25, wherein said Tat/Rev inhibitor is an anti-Tat/Rev shRNA.

Embodiment P27. The method of any one of embodiments P20-P26, wherein said nucleic acid further encodes a CCR5 inhibitor.

Embodiment P28. The method of embodiment P27, wherein said CCR5 inhibitor is an anti-CCR5 shRNA.

Embodiment P29. The method of any of embodiments P20-P28, wherein said contacting with an amount of 6-thioguanine (6TG) effective to cause hematopoietic toxicity in non-transduced T-cells is performed ex vivo.

Embodiment P30. A method of selecting for CAR T-cells from a population of transduced and non-transduced T-cells in a subject undergoing CAR-T therapy, said method comprising: administering a nucleic acid encoding a HPRT inhibitor and a CAR to said subject, thereby forming said population of transduced and non-transduced T-cells; administering 6-thioguanine (6TG) to said subject in an amount effective to cause hematopoietic toxicity in non-transduced T-cells, thereby resulting in selection of CAR T-cells.

Embodiment P31. The method of embodiments P30, wherein said nucleic acid is administered intravenously to said subject.

Embodiment P32. The method of embodiment P30 or P31, wherein said nucleic acid encodes an anti-HIV CAR.

Embodiment P33. The method of any one of embodiments P30-32 wherein said CAR comprises: (i) an antibody region comprising a heavy chain variable domain and a light chain variable domain, wherein said heavy chain variable domain comprises the sequence of SEQ ID NO:4, and wherein said light chain variable domain comprises the sequence of SEQ ID NO: 5; and (ii) a transmembrane domain.

Embodiment P34. The T-cell of any one of embodiments P30-P32, wherein said CAR comprises: (i) an antibody region comprising a heavy chain variable domain and a light chain variable domain, wherein said heavy chain variable domain comprises the sequence of SEQ ID NO:6, and wherein said light chain variable domain comprises the sequence of SEQ ID NO: 7; and (ii) a transmembrane domain

Embodiment P35. The method of any one of embodiments P30-P34, wherein said nucleic acid further encodes a Tat/Rev inhibitor.

Embodiment P36. The method of embodiment P35, wherein said Tat/Rev inhibitor is an anti-Tat/Rev shRNA.

Embodiment P37. The method of any one of embodiments P30-P36, wherein said nucleic acid further encodes a CCR5 inhibitor.

Embodiment P38. The method of embodiment P37, wherein said CCR5 inhibitor is an anti-CCR5 shRNA.

### Embodiments

Embodiment 1. A T-cell comprising: a nucleic acid comprising a sequence encoding an anti-HIV Chimeric Antigen Receptor (CAR); and i) a sequence encoding a Tat/Rev inhibitor, or ii) a sequence encoding a CCR5 inhibitor.

Embodiment 2. The T-cell of embodiment 1, wherein said nucleic acid comprises a sequence encoding a Tat/Rev inhibitor and a sequence encoding a CCR5 inhibitor.

Embodiment 3. The T-cell of embodiment 1 or 2, wherein said Tat/Rev inhibitor is an anti-Tat/Rev shRNA.

Embodiment 4. The T-cell of claim 3, wherein the anti-Tat/Rev shRNA comprises a sequence having at least 80% sequence identity to SEQ ID NO:10.

Embodiment 5. The T-cell of any one of embodiments 1-3, wherein said CCR5 inhibitor is an anti-CCR5 shRNA.

Embodiment 6. The T-cell of embodiment 5, wherein said anti-CCR5 shRNA comprises a sequence having at least 80% sequence identity to SEQ ID NO:9 or 11.

Embodiment 7. The T-cell of any of embodiments 1-6, wherein said nucleic acid further comprises a sequence encoding a hypoxanthine phosphoribosyl transferase (HPRT) inhibitor.

Embodiment 8. The T-cell of embodiment 7, wherein said HPRT inhibitor is an anti-HPRT shRNA.

Embodiment 9. The T-cell of embodiment 8, wherein the anti-HPRT shRNA comprises a sequence having at least 80% sequence identity to SEQ ID NO:12.

Embodiment 10. The T-cell of any one of embodiments 3, 5, or 8, wherein one or more of the anti-Tat/Rev shRNA, the anti-CCR5 shRNA, or the anti-HPRT shRNA comprises a wobble base.

Embodiment 11. The T-cell of any one of embodiments 1-10, wherein the anti-HIV CAR comprises: i) an antibody region capable of binding an HIV envelope protein; and ii) a transmembrane domain.

Embodiment 12. The T-cell of embodiment 11, wherein the HIV envelope protein is gp120 or gp41.

Embodiment 13. The T-cell of embodiment 11 or 12, wherein the antibody region comprises a heavy chain variable domain comprising the sequence of SEQ ID NO:4 and a light chain variable domain comprising the sequence of SEQ ID NO:5.

Embodiment 14. The T-cell of embodiment 11 or 12, wherein the antibody region comprises a heavy chain variable domain comprising the sequence of SEQ ID NO:6 and a light chain variable domain comprising the sequence of SEQ ID NO:7.

Embodiment 15. A T-cell comprising a nucleic acid comprising a sequence encoding an Anti-HIV Chimeric Antigen Receptor (CAR) and a sequence encoding an HPRT inhibitor.

Embodiment 16. The T-cell of embodiment 15, wherein said HPRT inhibitor is an anti-HPRT shRNA.

Embodiment 17. The T cell of embodiment 16, wherein the anti-HPRT shRNA comprises a sequence having at least 80% sequence identity to SEQ ID NO:12.

Embodiment 18. The T-cell of any one of embodiments 15-17, wherein the nucleic acid further comprises a sequence encoding a Tat/Rev inhibitor.

Embodiment 19. The T-cell of embodiment 18, wherein said Tat/Rev inhibitor is an anti-Tat/Rev shRNA.

Embodiment 20. The T-cell of embodiment 19, wherein the anti-Tat/Rev shRNA comprises a sequence having at least 80% sequence identity to SEQ ID NO:10.

Embodiment 21. The T-cell of any one of embodiments 15-20, wherein the nucleic acid further comprises a sequence encoding a CCR5 inhibitor.

Embodiment 22. The T-cell of embodiment 21, wherein said CCR5 inhibitor is an anti-CCR5 shRNA.

Embodiment 23. The T-cell of embodiment 22, wherein said anti-CCR5 shRNA comprises a sequence having at least 80% sequence identity to SEQ ID NO:9 or 11.

Embodiment 24. The T-cell of any one of embodiment 16, 19, or 22, wherein one or more of the anti-Tat/Rev shRNA, the anti-CCR5 shRNA, or the anti-HPRT shRNA comprises a wobble base.

Embodiment 25. The T-cell of any one of embodiments 15-24, wherein said anti-HIV CAR comprises: i) an antibody region capable of binding an HIV envelope protein; and ii) a transmembrane domain.

Embodiment 26. The T-cell of embodiment 25, wherein the HIV envelope protein is gp120 or gp41.

Embodiment 27. The T-cell of embodiment 25 or 26, wherein the antibody region comprises a heavy chain variable domain comprising the sequence of SEQ ID NO:4 and a light chain variable domain comprising the sequence of SEQ ID NO:5.

Embodiment 28. The T-cell of embodiment 25 or 26, wherein the antibody region comprises a heavy chain variable domain comprising the sequence of SEQ ID NO:6 and a light chain variable domain comprising the sequence of SEQ ID NO:7.

Embodiment 29. A pharmaceutical composition comprising the T-cell of any of embodiments 1-28 and a pharmaceutically acceptable excipient.

Embodiment 30. A nucleic acid comprising a sequence encoding an anti-HIV Chimeric Antigen Receptor (CAR); and i) a sequence encoding a Tat/Rev inhibitor, or ii) a sequence encoding a CCR5 inhibitor.

Embodiment 31. The nucleic acid of embodiment 30, comprising a sequence encoding a Tat/Rev inhibitor and a sequence encoding a CCR5 inhibitor.

Embodiment 32. The nucleic acid of embodiment 30 or 31, further comprising a sequence encoding a hypoxanthine phosphoribosyl transferase (HPRT) inhibitor.

Embodiment 33. A nucleic acid comprising a sequence encoding an anti-HIV Chimeric Antigen Receptor (CAR) and a sequence encoding a hypoxanthine phosphoribosyl transferase (HPRT) inhibitor.

Embodiment 34. The nucleic acid of embodiment 33, further comprising a sequence encoding a Tat/Rev inhibitor.

Embodiment 35. The nucleic acid of embodiment 33 or 34, further comprising a sequence encoding a CCR5 inhibitor.

Embodiment 36. A method of treating an HIV-infected subject in need thereof, said method comprising administering to said subject an effective amount of the T-cell of any one of embodiments 1-28.

Embodiment 37. The method of embodiment 36, wherein the T-cell is obtained from the subject.

Embodiment 38. The method of embodiment 36, wherein the T-cell is obtained from a donor who is not infected with HIV.

Embodiment 39. The method of any one of embodiments 36-38, wherein the T-cells are administered intravenously.

Embodiment 40. A method of treating an HIV-infected subject in need thereof, said method comprising: i) contacting a population of T-cells with a nucleic acid, thereby forming a population of transduced and non-transduced T-cells, wherein the nucleic acid comprises a sequence encoding an anti-HIV Chimeric Antigen Receptor (CAR) and a sequence encoding a HPRT inhibitor; and ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells; and ii) administering to said subject an effective amount of said population of selected transduced T-cells.

Embodiment 41. The method of embodiment 40, wherein the population of selected transduced and non-transduced T-cells is contacted with the effective amount of 6TG or 6-MP a plurality of times.

Embodiment 42. The method of embodiment 40 or 41 wherein said HPRT inhibitor is anti-HPRT shRNA.

Embodiment 43. The method of embodiment 42, wherein the anti-HPRT shRNA comprises a sequence having at least 80% sequence identity to SEQ ID NO:12.

Embodiment 44. The method of any one of embodiments 40-43, wherein the nucleic acid further comprises a sequence encoding a Tat/Rev inhibitor.

Embodiment 45. The method of embodiment 44, wherein said Tat/Rev inhibitor is an anti-Tat/Rev shRNA.

Embodiment 46. The method of embodiment 45, wherein the anti-Tat/Rev shRNA comprises a sequence having at least 80% sequence identity to SEQ ID NO:10.

Embodiment 47. The method of any one of embodiments 40-46, wherein the nucleic acid further comprises a sequence encoding a CCR5 inhibitor.

Embodiment 48. The method of embodiment 47, wherein said CCR5 inhibitor is an anti-CCR5 shRNA.

Embodiment 49. The method of embodiment 48, wherein said anti-CCR5 shRNA comprises a sequence having at least 80% sequence identity to SEQ ID NO:9 or 11.

Embodiment 50. The method of any one of embodiments 42, 45, or 48, wherein one or more of the anti-HPRT shRNA, the anti-Tat/Rev shRNA, or the anti-CCR5 shRNA comprises a wobble base.

Embodiment 51. The method of any one of embodiments 40-50, wherein said anti-HIV CAR comprises: (i) an antibody region capable of binding an HIV envelope protein; and (ii) a transmembrane domain.

Embodiment 52. The method of embodiment 51, wherein the HIV envelope protein is gp120 or gp41.

Embodiment 53. The method of embodiment 51 or 52, wherein the antibody region comprises a heavy chain variable domain comprising the sequence of SEQ ID NO:4 and a light chain variable domain comprising the sequence of SEQ ID NO:5.

Embodiment 54. The method of any one of embodiments 40-53, wherein said population of T-cells is obtained from the subject.

Embodiment 55. The method of any one of embodiments 40-54, wherein said population of T-cells is obtained from a donor who is not infected with HIV.

Embodiment 56. The method of any one of embodiments 40-55, wherein the population of selected transduced T-cells are administered intravenously.

Embodiment 57. A method of selecting for T-cells comprising: i) contacting a population of T-cells with a nucleic acid, thereby forming a population of transduced T-cells and non-transduced T-cells, wherein the nucleic acid comprises a sequence encoding an anti-HIV Chimeric Antigen Receptor (CAR) and a sequence encoding a HPRT inhibitor; and ii) contacting the population of transduced T-cells and non-transduced T-cells with an amount of 6-thioguanine (6TG) or 6-mercaptopurine (6-MP) effective to cause cell death in the non-transduced T-cells, thereby forming a population of selected transduced T-cells.

Embodiment 58. The method of embodiment 57, wherein the population of T-cells is contacted with the effective amount of 6TG or 6-MP a plurality of times.

Embodiment 59. The method of embodiment 57 or 58, wherein said HPRT inhibitor is anti-HPRT shRNA.

Embodiment 60. The method of embodiment 59, wherein the anti-HPRT shRNA comprises a sequence having at least 80% sequence identity to SEQ ID NO:12.

Embodiment 61. The method of any one of embodiments 57-60, wherein the nucleic acid further comprises a sequence encoding a Tat/Rev inhibitor.

Embodiment 62. The method of embodiment 61, wherein said Tat/Rev inhibitor is an anti-Tat/Rev shRNA.

Embodiment 63. The method of embodiment 62, wherein the anti-Tat/Rev shRNA comprises a sequence having at least 80% sequence identity to SEQ ID NO:10.

Embodiment 64. The method of any one of embodiments 57-63, wherein the nucleic acid further comprises a sequence encoding a CCR5 inhibitor.

Embodiment 65. The method of embodiment 64, wherein said CCR5 inhibitor is an anti-CCR5 shRNA.

Embodiment 66. The method of embodiment 65, wherein said anti-CCR5 shRNA comprises a sequence having at least 80% sequence identity to SEQ ID NO:9 or 11.

Embodiment 67. The method of any one of embodiments 60, 62, or 65, wherein one or more of the anti-Tat/Rev shRNA, the anti-CCR5 shRNA, or the anti-HPRT shRNA comprises a wobble base.

Embodiment 68. The method of any one of embodiments 57-67, wherein said anti-HIV CAR comprises: i) an antibody region capable of binding an HIV envelope protein; and ii) a transmembrane domain.

Embodiment 69. The method of embodiment 68, wherein the HIV envelope protein is gp120 or gp41.

Embodiment 70. The method of embodiment 68 or 69, wherein the antibody region comprises a heavy chain variable domain comprising the sequence of SEQ ID NO:4 and a light chain variable domain comprising the sequence of SEQ ID NO:5.

### EXAMPLES

### Example 1: Summary of Exemplary Experiments

Challenges associated with HIV need to be addressed in order to make CAR T-cell immunotherapy an efficacious treatment. First, HIV can infect the CD4+ CAR T-cells, and recent studies have shown that a lack of CD4+ population is related to a decrease in in vivo persistence and efficacy of CAR therapy. To address this issue, Applicant introduced a short hairpin RNA (shRNA) against CCR5, a crucial protein for HIV entry, into the CAR construct. This shRNA demonstrated sufficient knockdown of CCR5 in primary T-cells and prevented viral entry of R5-tropic virus in T-cell reporter line. Although this strategy will prevent viral uptake, this shRNA does not address the issue of HIV latency and replication, as CD4+ T-cells are a reservoir of HIV, which can be reactivated during ex vivo expansion. To address this issue, an shRNA was introduced against Tat/Rev, essential proteins for HIV replication, into the CAR construct. This shRNA protected against R5- and X4-tropic viral replication in a T-cell reporter line as well as suppressed viral reactivation in HIV donor CAR T-cells. Collectively, the combination of these two shRNAs in an anti-HIV CAR construct were capable of increasing expansion potential and retain memory phenotype when compared to unprotected CAR T-cells. However, both protected and unprotected anti-HIV CAR T-cells from HIV infected donors demonstrated decreased efficacy when compared to uninfected donor cells. This this lack of efficacy was attributed to HIV antigen overstimulation from non-transduced T-cells. Antigen expressed or released from the non-transduced T-cells stimulates the CAR T-cells throughout the culture leading to an exhausted/anergic CAR T-cell population. To address this issue of non-transduced T-cells, an shRNA against hypoxanthine-guanine phosphoribosyltransferase (HPRT) was added, which results in the CAR T-cells becoming resistant to 6-thioguanine (6TG). 6TG is an antimetabolite that is processed by HPRT and integrated into the DNA ultimately leading to cell death. Studies provided here show that repression of HPRT in CAR T-cells protects the cells from 6TG-induced cell death allowing for efficient enrichment of anti-HIV protected anergic resistant CAR T-cells. Notably, the repression of HPRT did not appear to impact CAR T-cell functionality. Collectively, the strategy presented here may significantly improve antiviral therapy of CAR T-cells in people living with HIV/AIDs.

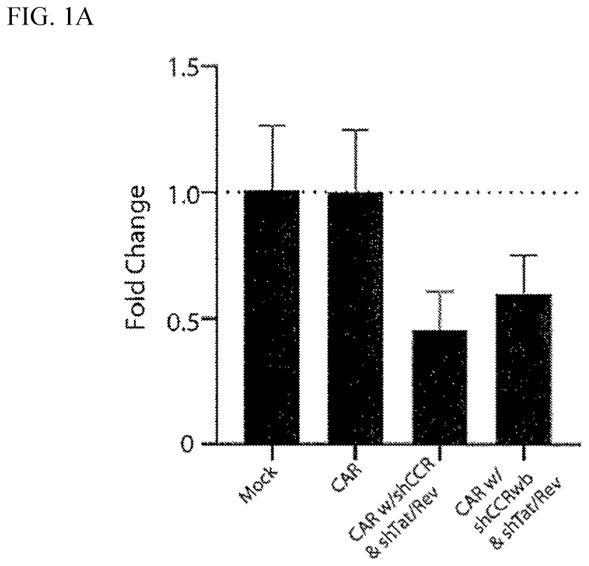

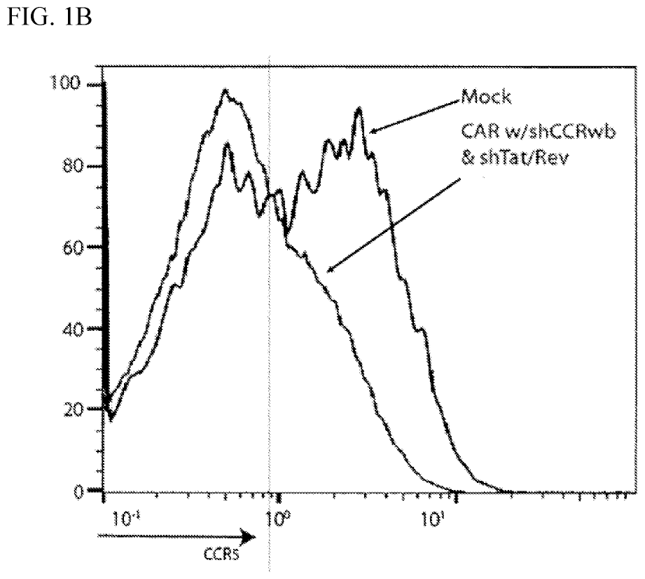

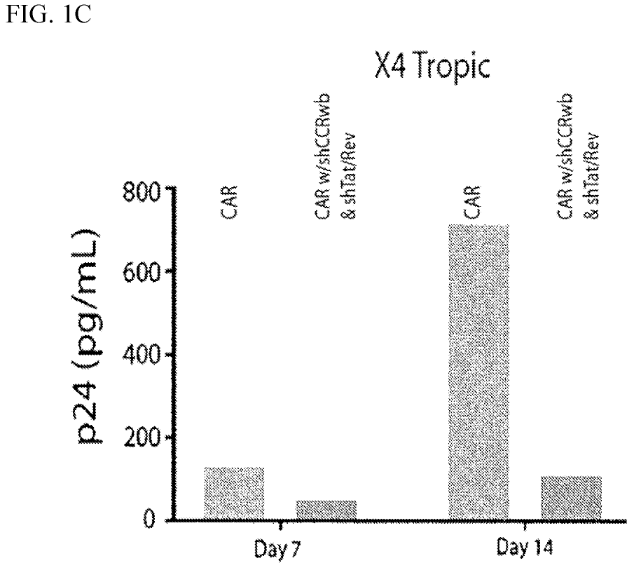

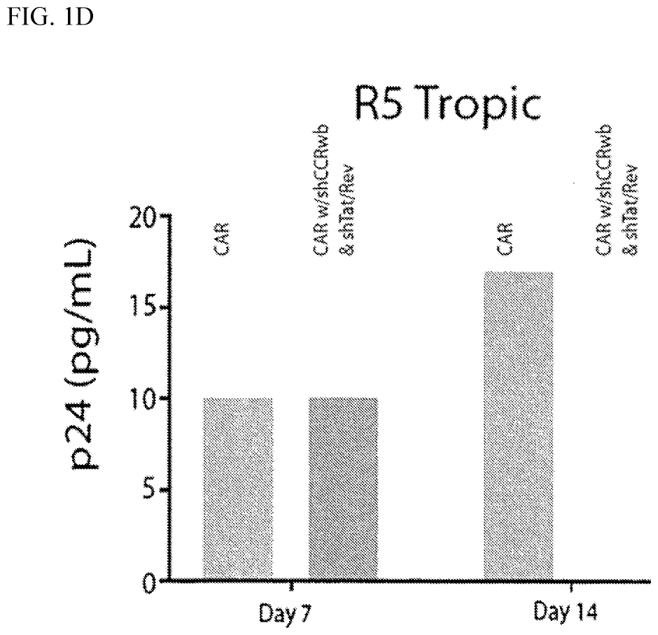

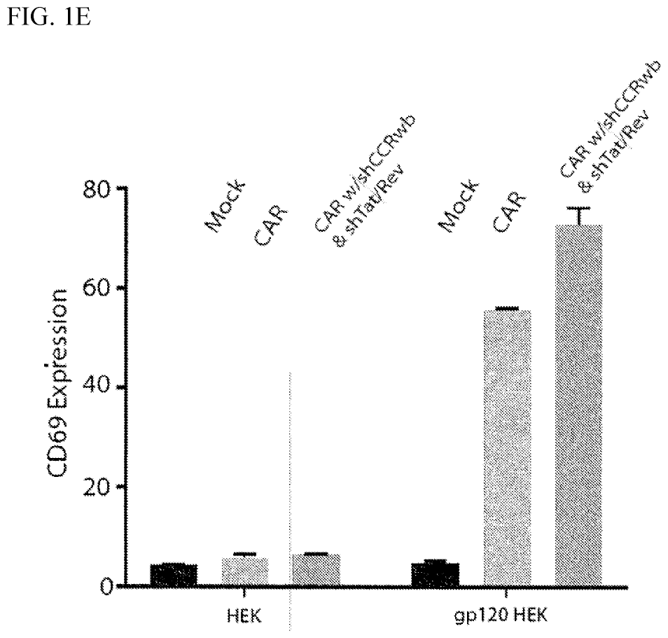

Applicant demonstrates herein that a protection cassette containing functional tRNA pol III expressed shRNA can knockdown both HIV Tat/Rev and CCR5 (FIGS. 1A-1B). It is shown that this protection modality can protect cells from HIV infection of both the X4 and R5 tropic variants, as shown by decreased levels of HIV antigen p24 (FIGS. 1C-1D). Applicant further found that this anti-HIV CAR protection cassette does not affect CAR function, as indicted by high levels of CD69 expression in T-cells transduced with the protection anti-HIV CAR construct (FIG. 1E). This indicates that the transduced T-cells maintain potent cytotoxicity and ability to produce proinflammatory cytokines. It was further confirmed that the constructs are capable of protecting CAR T-cells by knocking out CCR5 to prevent HIV infection and Tat/Rev to prevent HIV replication. Applicant demonstrates herein that introduction of protection anti-HIV CAR constructs into HIV-patient T-cells produces T-cells capable of suppressing HIV reactivation and reviving T-cell growth potential. The T-cells are further capable of retaining central memory markers and cytotoxic efficacy.

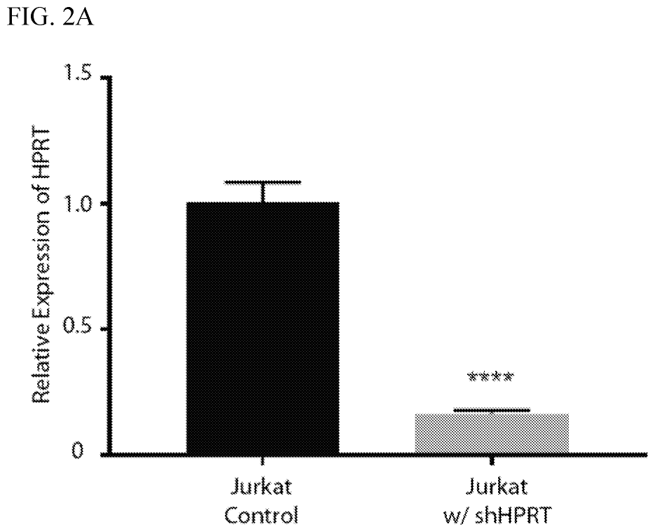

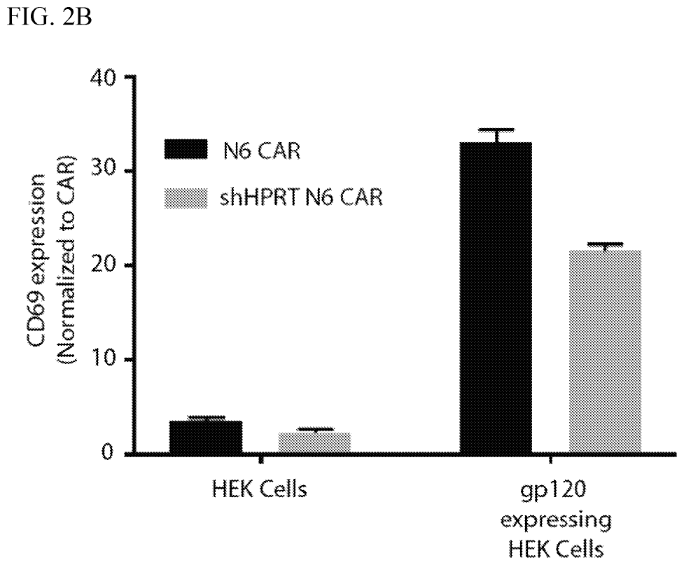

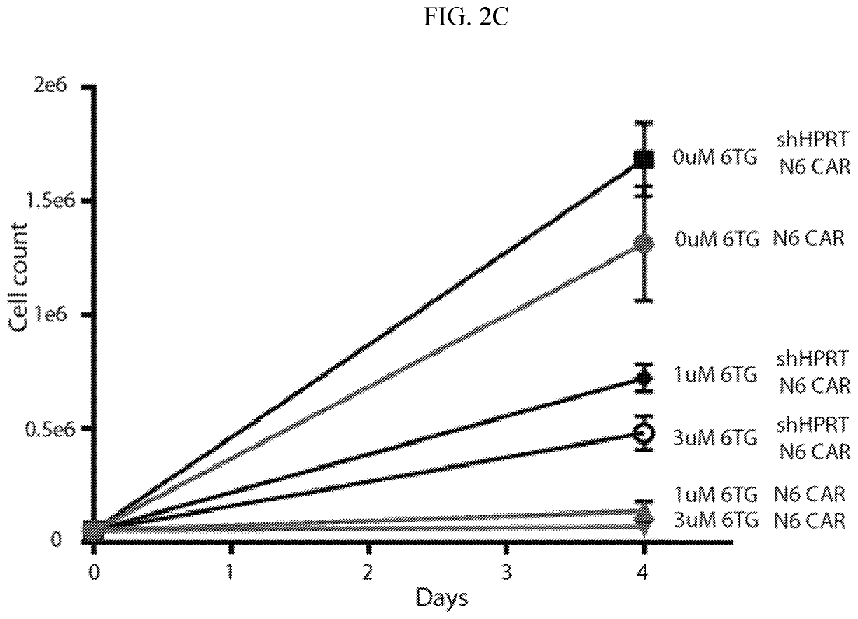

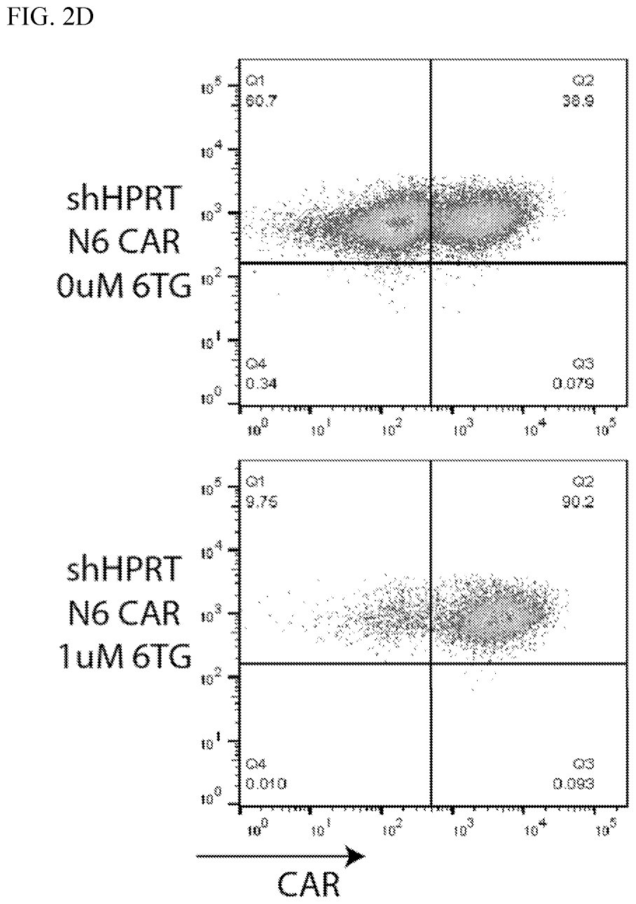

Moreover, described herein is an approach that relies on shRNA-mediated knockdown of hypoxanthine phosphoribosyl transferase (HPRT), the enzyme required for metabolizing purine analogs like 6-thioguanine (6TG) and mercaptopurine (6-MP) (1). This strategy provides drug resistance without the introduction of a foreign protein which could in theory lead to immunologic elimination of the transduced cells. Applicant demonstrates here specific, selective enrichment of CAR T-cells by embedding an HPRT shRNA into the anti-HIV CAR lentiviral vector. and treatment of the lentiviral vector transduced cells with 6TG (FIGS. 2A-D). It was found that HPRT is robustly repressed by the anti-HPRT shRNA (FIG. 2A). Further, the anti-HIV CAR T-cell line containing the anti-HPRT shRNA retains similar functionality as it is activated by its target antigen, as shown by high levels of CD69 expression (FIG. 2B). Thus, 6TG eliminates cells that lack the anti-HPRT shRNA (FIG. 2C) and results in the selection of a population of cells containing >90% of anti-HIV CAR expressing cells (FIG. 2D).

Collectively, Applicant has developed a new method to chemically enrich for CAR T-cells, which is facile compared to sorting methods that require complicated and expensive reagents and equipment. This approach may ensure that substantially all of the cells utilized are CARs and not unmodified T-cells; thus this method may be used for CAR manufacturing. Notably, 6TG selection can also be used in vivo to expand CAR T-cells and therefore this technology could be applied to generating CAR T-cells by direct injection of lentiviral vectors into patient bone marrow, thereby circumventing the need to do ex vivo manipulation in the genesis of CAR T-cells. Such an outcome could drastically cut the costs of CAR T-cell treatments for patients and allow for a broader utility of this technology.

### Example 2: Protecting Anti-HIV CAR T-Cells by Short Hairpin RNAs

While previously developed conditionally-replicating CARs showed evidence of protection of CD4 T-cells as well as mobilization of the CAR construct in the presence of HIV, more testing is necessary to understand the dynamics of this therapy in vivo. Further, prior anti-HIV CAR therapies attempted to prevent HIV infection by knocking out crucial proteins, such as CCR5¹⁴⁹, or adding fusion inhibitors to the CAR T-cell^{151,152,155} However, these methods would only work if anti-HIV CAR T-cell therapy used allogeneic T-cells. Thus, preventing and suppressing HIV infection in CAR T-cells would be clinically relevant. To develop an autologous anti-HIV CAR T-cells therapy, Applicant demonstrates herein that there is a need to block both HIV infection and HIV reactivation.

To address HIV infection and reactivation, Applicant used shRNAs against CCR5 and Tat/Rev. The shRNA against CCR5 would be used to knockdown CCR5 expression to prevent the infection of HIV virus^{178,179,180; 181} However, this shRNA would only protect against R5-tropic virus, CCR5 centric^{178,179}, and not X4-tropic virus, CXCR4 centric¹⁷⁹. The anti-Tat/Rev shRNA target would alleviate the issue of non-protection against X4-tropic virus by preventing all HIV reactivation by targeting the Tat and Rev proteins, which are crucial for HIV replication^{182,183,184, 185} Because the combination of an anti-CCR5 and an anti-Tat/Rev shRNA may be useful for preventing both HIV infection and replication, respectively, Applicant believed that they may alleviate HIV reactivation in autologous donors for CAR T-cells.

### Results

Optimizing shCCR shTat/Rev CAR Construct

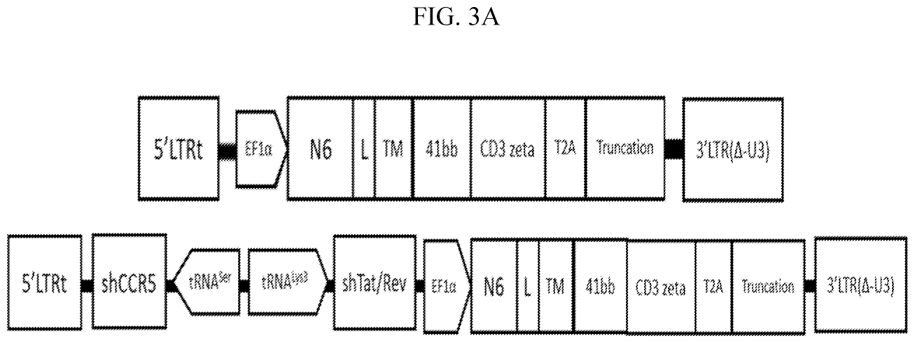

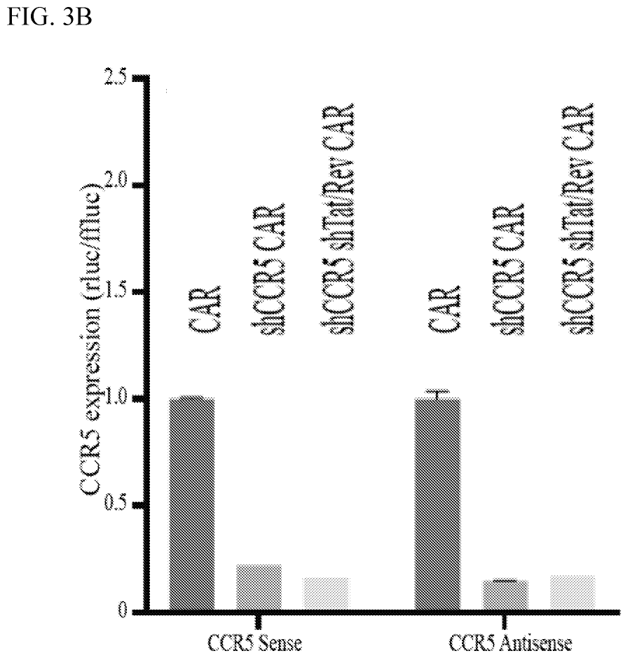

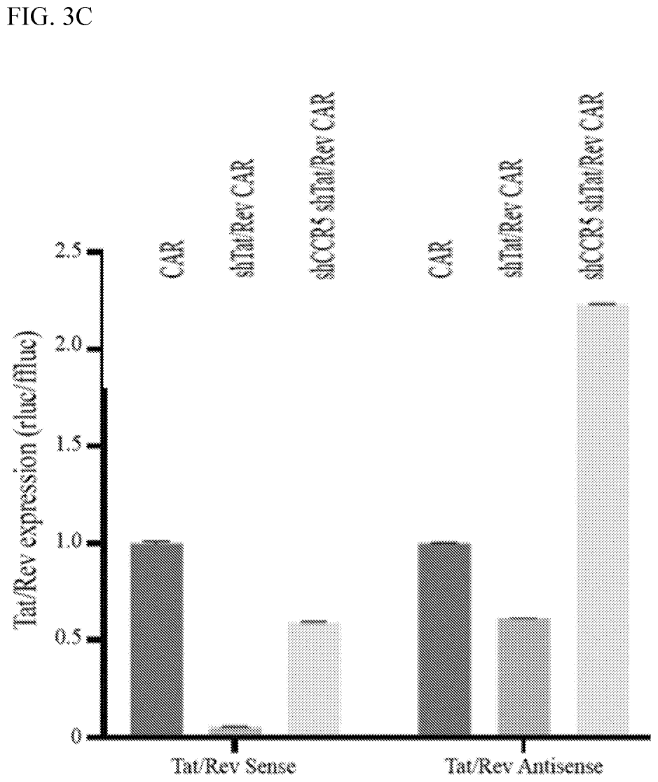

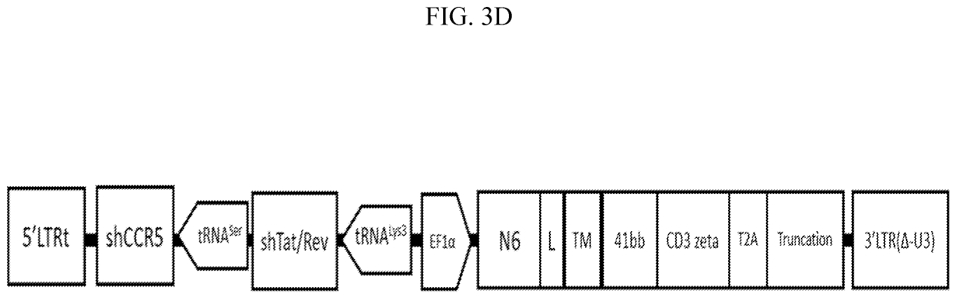

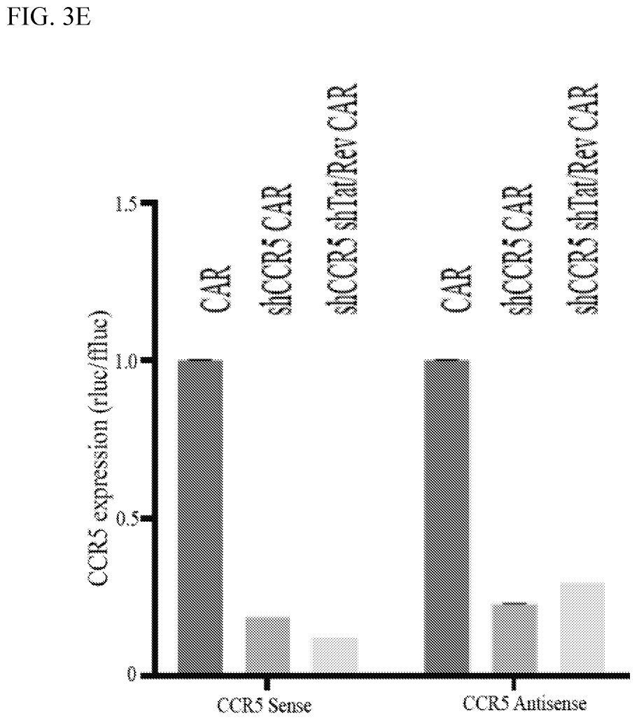

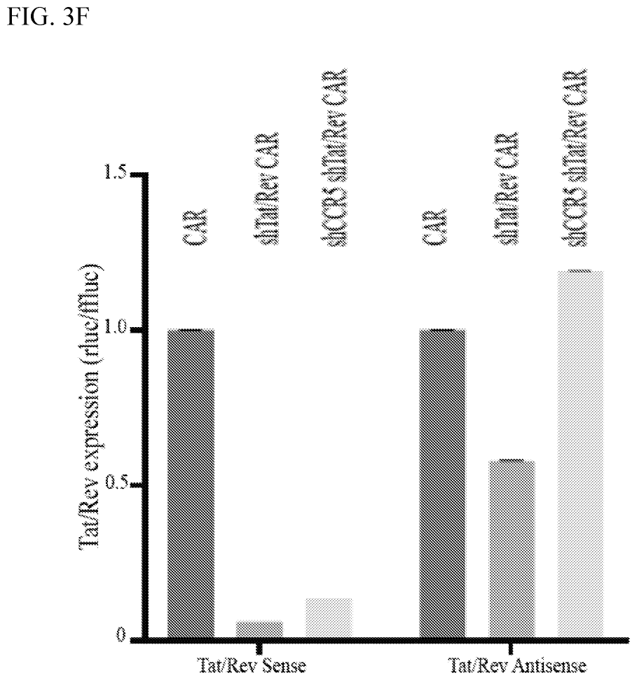

Previous studies have shown that when designing a lentivirus construct that contains shRNAs there are several things to take into consideration. First, shRNAs need to be expressed from polymerase three (Pol III) promoter¹⁸⁶. Since the CAR construct is expressed from an EF1α promoter, a polymerase two (Pol II) promoter, it became apparent that the shRNAs needed to be expressed from their own Pol III promoter. Previous studies also have shown that tRNA promoters, when compared to stronger Pol III promoters like U6 and H1, have similar activity without toxicity¹⁸⁶. Second, it has been reported that having shRNAs expressed in the sense direction could have auto-targeting, which could have potential detrimental effects on titer¹⁸⁷. Operating under these considerations, two constructs were designed, both of which had the shCCR5 expressed under a serine tRNA promoter in the antisense direction, while the shTat/Rev was expressed under a lysine 3 tRNA promoter but expressed in either the sense (FIG. 3A) or the antisense (FIG. 3D) direction. Functionality of the construct was determined through dual luciferase knockdown assay. The CCR5 or Tat/Rev targets, both sense and antisense, were expressed in a Psi-check plasmid. The sense strand represents target knockdown and the antisense represents self-knockdown¹⁸⁷. For the construct described in FIG. 3A (e.g. antisense shCCR5 sense shTat/Rev anti-HIV CAR), Applicant observed that shCCR5 was capable of knocking down the sense and antisense strand (FIG. 3B), while shTat/Rev was incapable of sufficiently knocking down either target (FIG. 3C). For the construct described in FIG. 3D, results showed that shCCR5 was capable of knocking down both sense and antisense strand (FIG. 3E), while shTat/Rev only knocked down the sense strand (FIG. 3F). For these experiments, anti-HIV CAR without shRNAs were used as negative controls and constructs with single shRNA were used as positive control. Applicant decided to proceed with the construct described in FIG. 3D (e.g. antisense shCCR5 antisense shTat/Rev anti-HIV CAR), with both shRNAs in the antisense direction, as it had functioned for both CCR5 and Tat/Rev. However Applicant needed to alleviate the capability of shCCR5 for targeting the antisense strand or risk lowering lentiviral production.

To alleviate this issue, wobble bases were introduced, guanine or uracil, to the 3′ end of the sense strand^{188, 189}. This reduced self-targeting by lowering the thermostability at the 5′ end of the antisense strand thus increasing strand selection in the RISC complex¹⁸⁹. Compared to the original design (FIG. 4A), a uracil was introduced in position 16 and a guanine in positions 18 and 21 of the sense strand (FIG. 4B). When comparing the shCCR5w shTat/Rev CAR to the previous construct in the knockdown assay, it was observed that for both targets, CCR5 and Tat/Rev, the shCCR5w shTat/Rev CAR construct will only target the sense strand, while leaving the antisense strand unaffected (FIGS. 4C-4D). This shows that this construct is capable of targeting both CCR5 and Tat/Rev without impacting lentivirus titers. From this point, it is noted that shCCR5w shTat/Rev anti-HIV CAR may be also be referred to as “protection anti-HIV CAR” or “protection CAR”.

The Protection Anti-HIV CAR Protects T-Cells from HIV without Impacting T-Cell Functionality

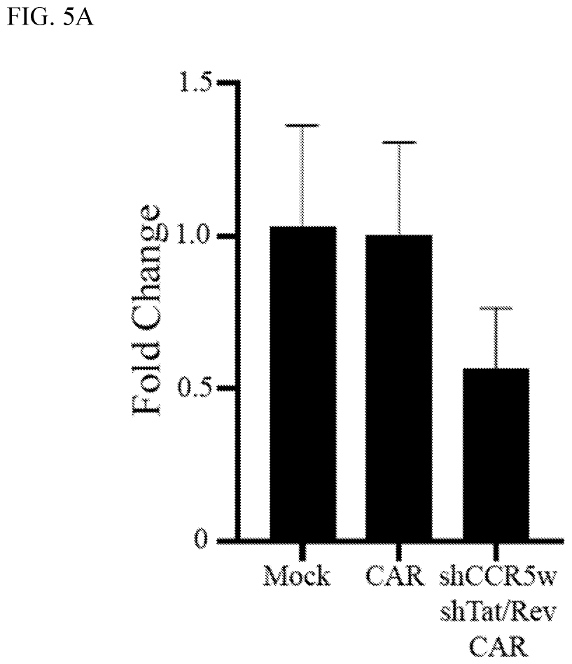

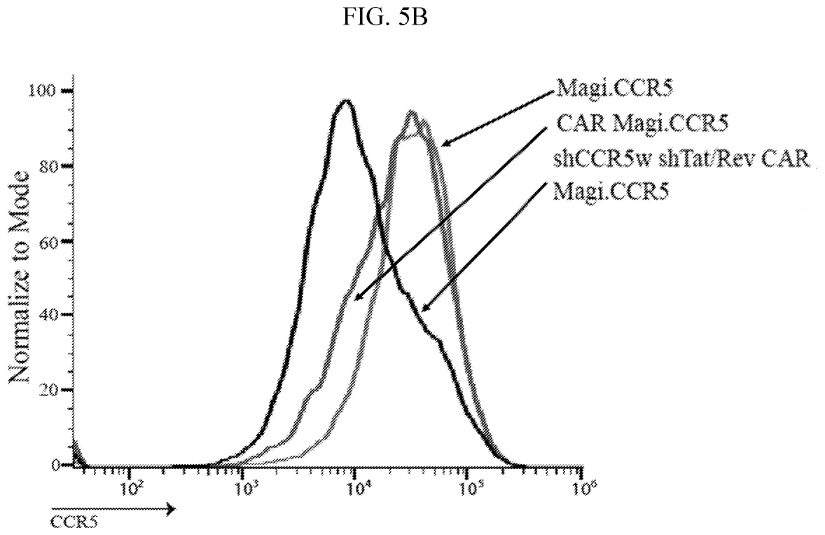

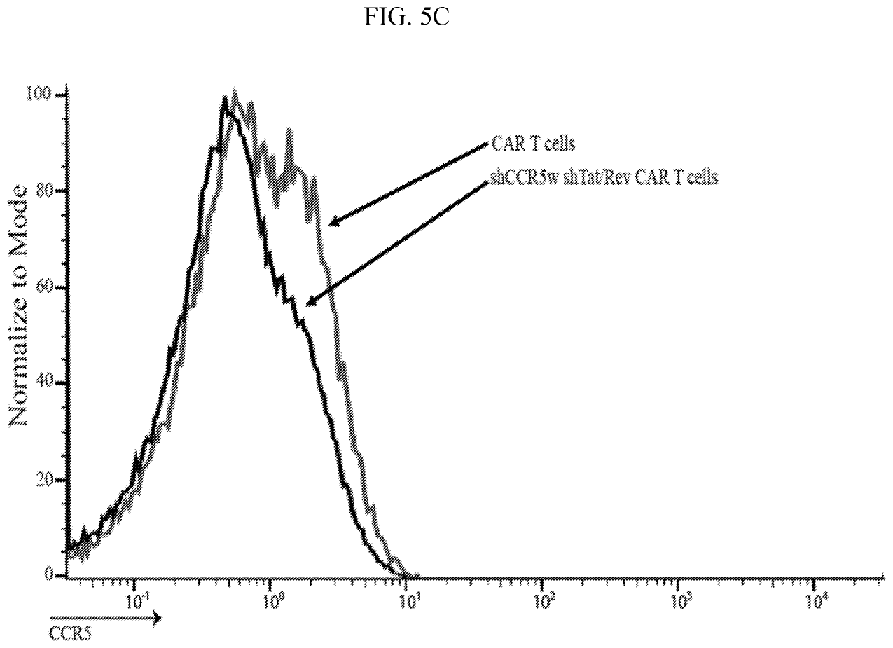

Next, Applicant determined whether the shRNAs were functional. To test the shCCR5, Applicant transfected the protection and conventional CAR into Magi reporter line expressing CCR5 (Magi.CCR5). It was observed through both qualitative PCR and flow cytometry that shCCR5w was successful in knocking down CCR5 (FIGS. 5A-5B). To confirm knock-down of CCR5, Applicant transduced T-cells with protection and conventional anti-HIV CAR constructs and analyzed CCR5 surface expression. It was confirmed that the protection CAR is capable decreasing CCR5 expression (FIG. 5C).

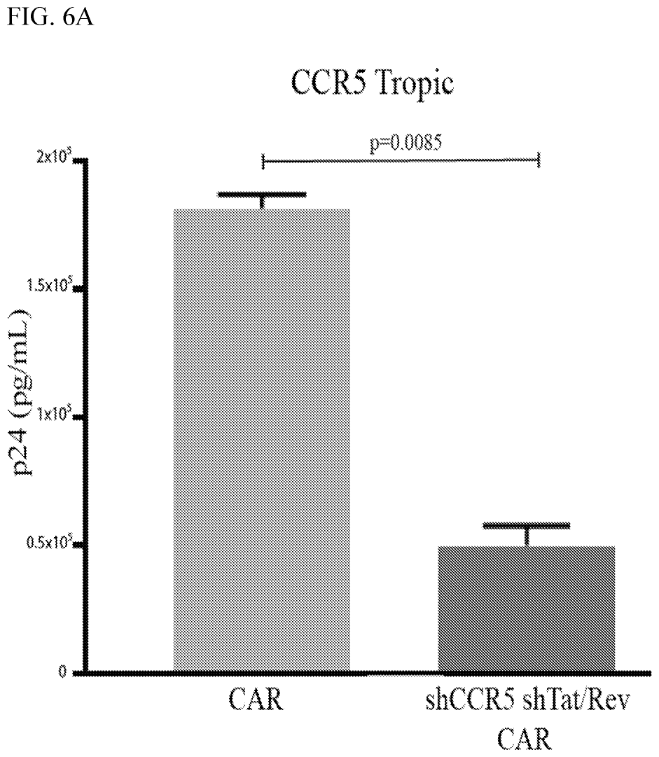

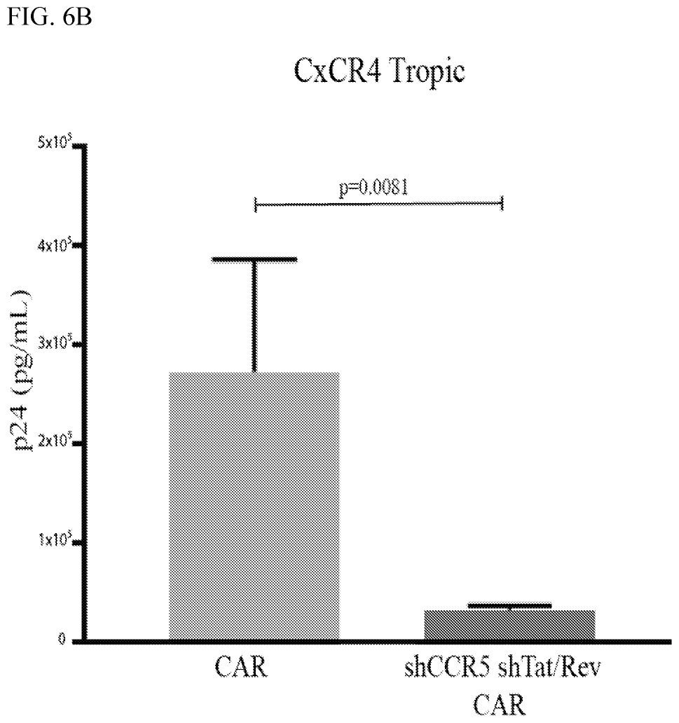

To test the functionality of shTat/Rev, CCR5-expressing Jurkat cells (Jurkat.CCR5) were transduced with protection or conventional CAR then infected with either R5- (Yu2) or X4- (LAI) tropic HIV. Following a 2 week incubation, Applicant analyzed the p24 level in the culture supernatant (FIGS. 6A-6B). A significant decrease in free virus was observed for both tropisms for HIV for protection CAR when compared to conventional CAR. This suggests that both anti-CCR5 and anti-Tat/Rev shRNAs are functional, and further that the protection CAR is capable of protecting anti-HIV CAR T-cells from both tropisms of HIV.

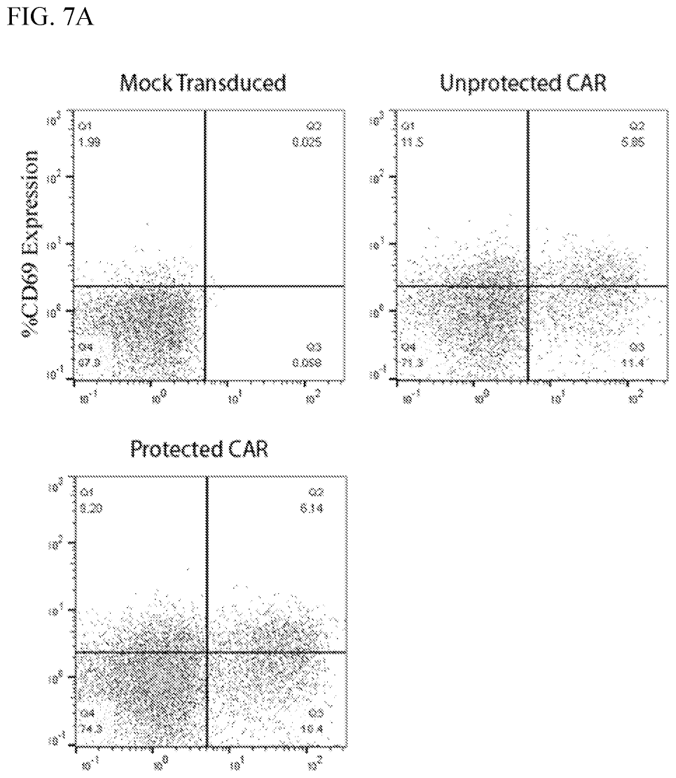

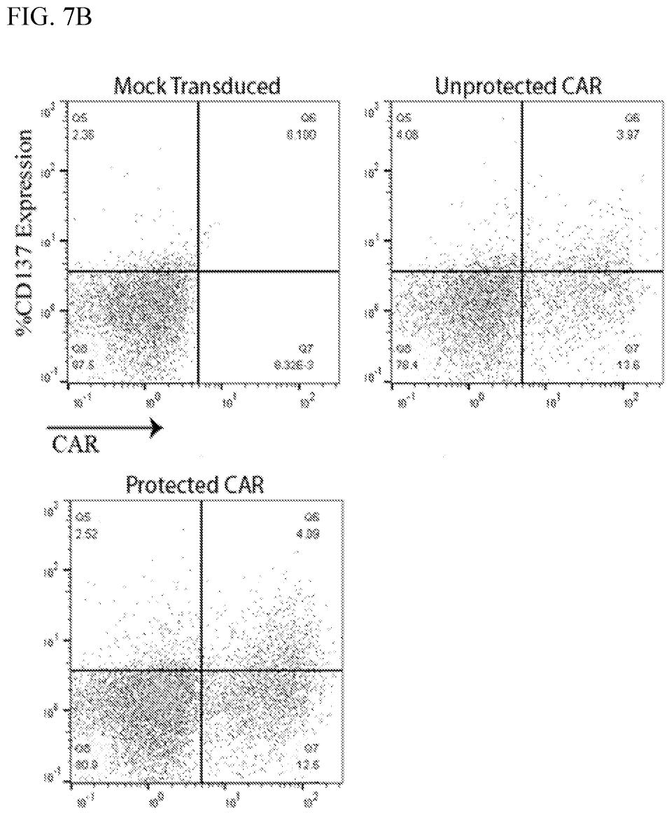

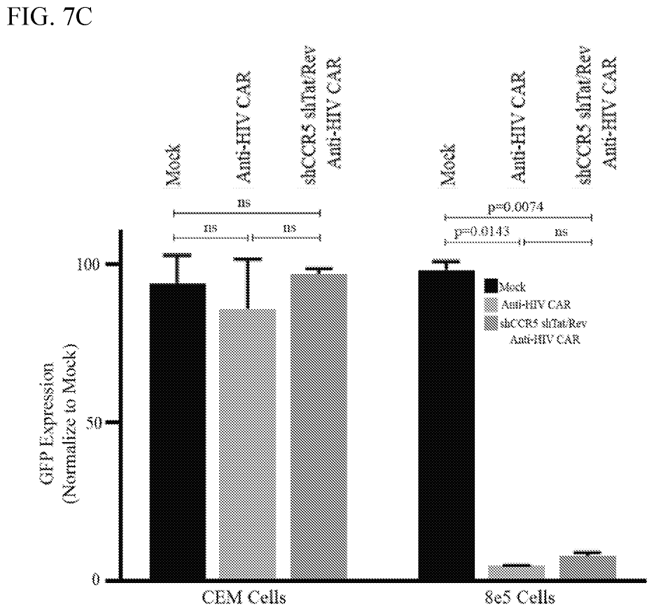

While Applicant demonstrated that the shRNAs are functional, confirmation that the shRNAs do not impact CAR functionality was necessary. To test activation, Applicant co-cultured protection or conventional anti-HIV CAR with either gp160-expressing or parental HEK293 cells (n=3)¹⁷⁶. Following a 24 hour incubation, both CD69 and CD137 expression^{190, 191} were analyzed. Applicant observed that both protection and conventional anti-HIV CAR T-cells cultured with HEK293.gp160 expressed equivalent levels of CD69 and CD137 (FIGS. 7A-7B). The results indicate that the shRNA do not effect anti-HIV CAR functionality.

Next, to determine cytotoxic functionality, protection or conventional anti-HIV CARs were co-cultured with either 8e5.GFP cells, CEM cells that contain defective HIV-1 provirus¹⁷⁷ or parental CEM.GFP at a ratio of 1 CAR T-cell. 4 target cells (n=3). Both protection and conventional anti-HIV CAR T-cells had greater than 90% cytotoxicty against 8e5.GFP cells when compared to mock-transduced T-cells (FIG. 7C). These data show that shRNAs in the protection anti-HIV CAR had no significant impact on CAR T-cell functionality.

### The Protection Anti-HIV CAR Resists HIV Reactivation and Reinvigorates HIV-Donor T-Cell Functionality.

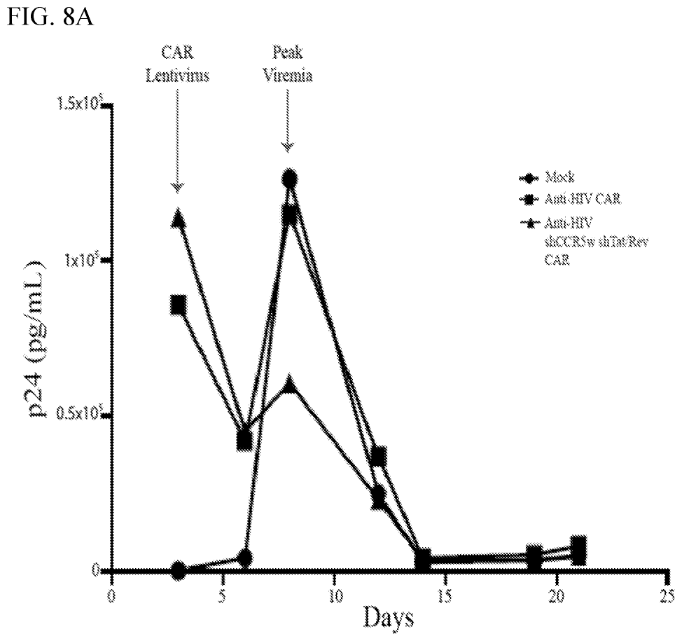

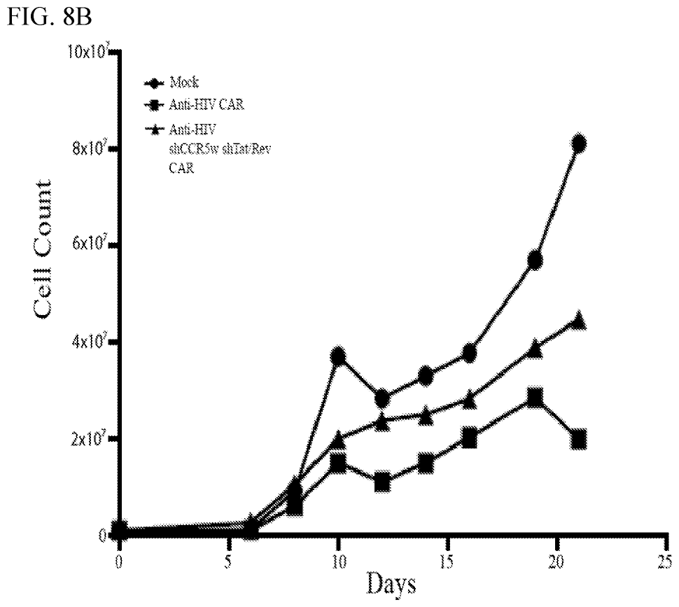

To interrogate how this protection anti-HIV CAR would influence autologous HIV-patient T-cells, T-cells taken from HIV patients were transduced with protection or conventional anti-HIV CAR construct (n=1). To determine if protection anti-HIV CAR was capable of suppressing HIV reactivation in the transduced T-cells, Applicant collected supernatant from the T-cell cultures for analysis of p24 levels over the course of the cell culture. A decrease in p24 levels was observed for HIV patient T-cells transduced with protection anti-HIV CAR construct as compared to HIV patient T-cells transduced with anti-HIV CAR construct or mock-transduced T-cells (FIG. 8A). Furthermore, superior suppression of p24 levels was observed at day nine, which appeared to be the day of peak viremia.

Next, Applicant analyzed the cell growth over the course of culturing, and the phenotype of cells transduced with either protection and conventional anti-HIV CAR lentivirus. For cell growth, conventional anti-HIV CAR T-cells had a significant lack of cell growth when compared to mock-transduced cells (FIG. 8B). Protection anti-HIV CAR transduced cells, however, had a revival of cell growth potential (FIG. 8B).

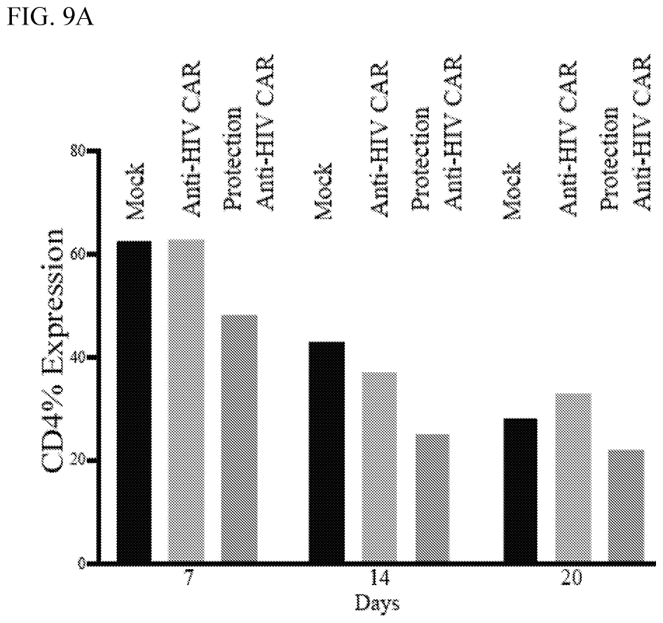

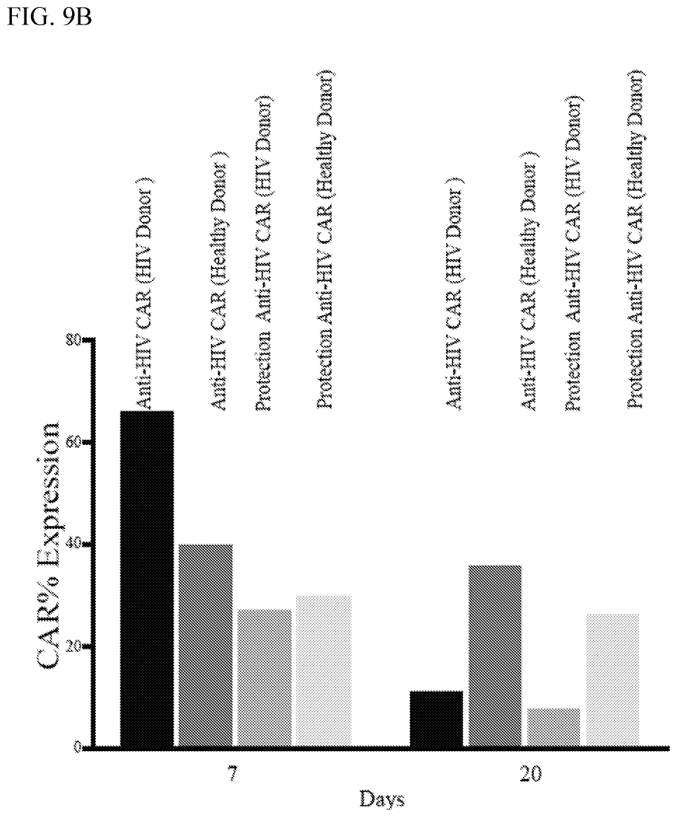

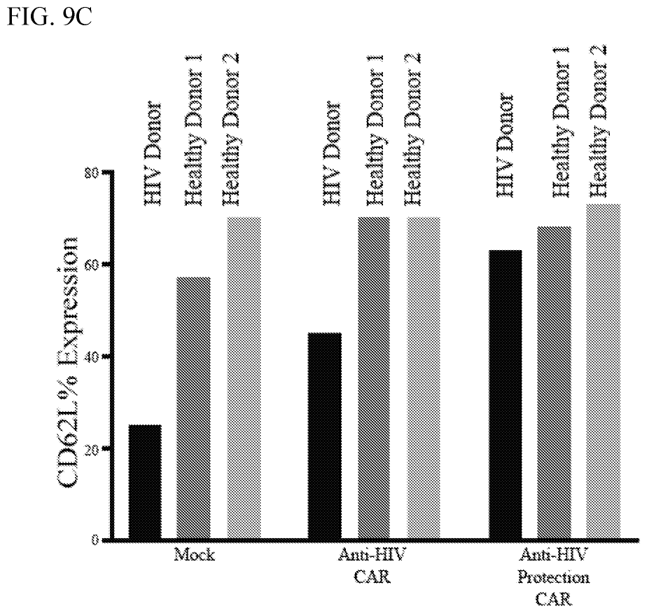

Phenotype of transduced T-cells were analyzed by measurement of CD4/CD8 ratio, CAR percentage, and memory markers. By analyzing the CD4/CD8 ratio over the course of cell culture, it was shown that protection anti-HIV CAR cells had a similar decrease in CD4 percentage over the course of culturing compared to conventional anti-HIV CAR cells (FIG. 9A). Furthermore, CAR expression decreased throughout culturing time, which was not observed in healthy-donor T-cells (FIG. 9B). This suggests that the HIV-infected CAR T-cells influenced CD4 and CAR expression levels over the course of culturing. Finally, the central memory marker (CD62L) was interrogated. Only protection anti-HIV CAR lentivirus transduced cells were capable of retaining similar CD62L expression levels in T-cells taken from HIV patients as compared to T-cells taken from healthy donors (FIG. 9C).

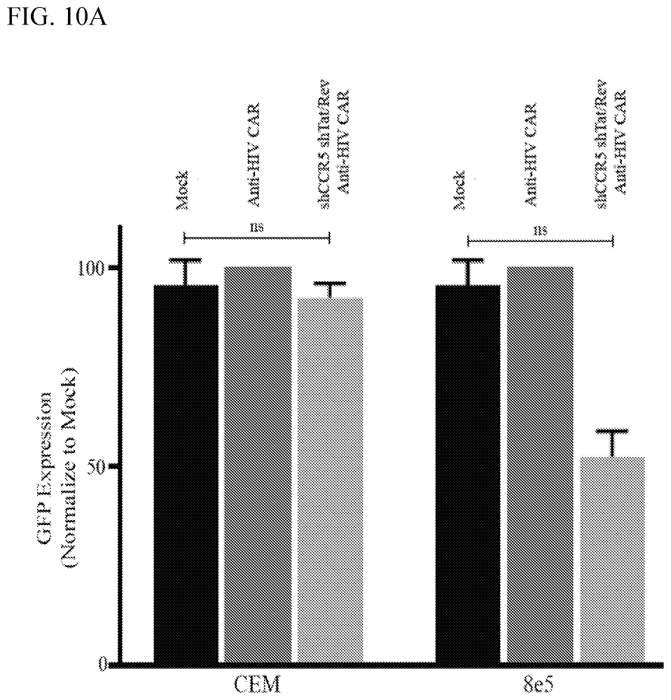

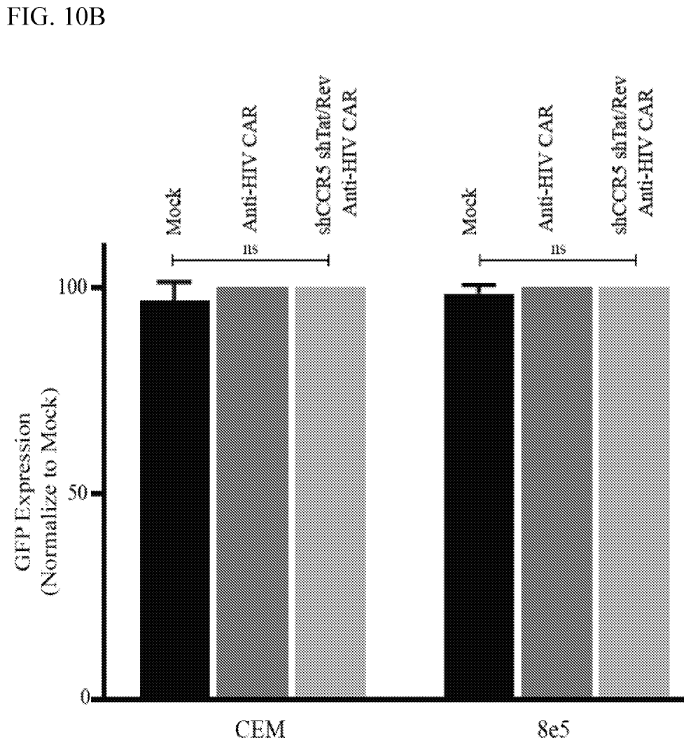

Lastly, Applicant analyzed the cytotoxic functionality of the protection anti-HIV CAR. Protection or conventional anti-HIV CAR transduced cells from a HIV patient were co-cultured with either 8e5.GFP cells or CEM.GFP cells. The cells were cultured at two ratios: the first ratio tested was 1 anti-HIV CAR T-cell:1 target cell and the second ratio tested was 1 anti-HIV CAR T-cell:4 target cells. The first ratio was designed to be more preferential for T-cells from HIV patients, while the second ratio was developed to be preferential for T-cells from healthy donors. For the first ratio, protection anti-HIV CAR transduced cells had 50% cytotoxic efficacy against 8e5.GFP cells, while conventional anti-HIV CAR had no efficacy (FIG. 10A). For the second ratio, neither protection nor conventional CAR had significant cytotoxic effects against 8e5.GFP cells (FIG. 10B). For both ratios the T-cells were taken from the same donor. Although the T-cells were taken from a single donor, this suggests that the protection anti-HIV CAR is capable of maintaining CAR functionality in HIV-donors but is incapable of reviving autologous T-cells from HIV-donors to the level of healthy donors.

### Conclusion

This new anti-HIV modality can be incorporated into any CAR platform and is amenable to being used with the 6TG or 6MP selection scheme. This method is contemplated to provide an effective means to generating a substantially pure anti-HIV CAR product that is protected from HIV infection and is therefore more effective for use against HIV infected cells since the CARs are protected from further rounds of infection. Significantly, this research demonstrates utility of CAR therapy in HIV patients, who were previously excluded from such studies¹⁹².

### Example 3: Enriching Anti-HIV CAR T-Cells by Short Hairpin RNAs

### Introduction

Applicant has shown there is a necessity for addressing HIV reactivation in CAR T-cells. Described herein are methods for altering the CAR construct to address this issue. However, due to the unlikelihood for 100% CAR transduction, the non-transduced CD4 T-cells become a secondary issue. The infected CD4 T-cells, once reactivated, can present gp120 antigen which can engage and activate anti-HIV CAR T-cells, which could lead to overactivation and anergy^{193, 194}. Furthermore, uninfected, unprotected CD4 T-cells can become infected, ex vivo, leading to more antigen presentation and potentially further overactivation and anergy.

Two methodologies may be employed to enrich for CAR T-cells. The first method is using antibodies that will bind to the CAR T-cells specifically. One such method, described by Casucci et al., demonstrated this by including nerve-growth-factor receptor (NGFR) in the spacer domain. This specific spacer can be labeled with antibodies and further separated out by using magnetic beads¹⁹⁵. However, additional processing added to the CAR T-cell manufacturing not only adds to the cost to the production but could add unintended stress to the CAR T-cells. The second method is to remove the non-transduced T-cells from the culture. However, this methodology would be difficult as there are no markers separating CAR T-cells from non-transduced T-cells.

Other methods for selection of CAR T-cells include implementation of a variety of drug resistant genes¹⁹⁶⁻²⁰² For CAR T-cells, Jonnalagadda et al. introduced mutated dihydrofolate reductase (DHFR) and inosine-5′-monophosphate dehydrogenase II (IMPDH2) to the CAR construct that made CAR T-cells resistant to methotrexate (MTX) and mycophenolate mofetil (MMF), respectively^{197, 203-205} While this construct was capable of enriching T-cells, both ex vivo and in vivo, the size of the proteins added considerable size to the total construct which could have deleterious effects on lentiviral packaging^{175, 197}.

The antimetabolite, 6-thioguanine (6TG), has been used clinically as a cancer therapy²⁰⁶. 6TG is a thiopurine that is enzymatically converted to 6-thioguanine monophosphate (6GMP) by hypoxanthine-guanine phosphorbosyl transferase (HPRT) and then further converted with kinase reductase to 6-diThioguanine triphosphate (6GTP)^{207, 208}. 6GTP is then incorporated into the DNA as a fraudulent base, which will lead to DNA damage and an arrest of the cell cycle^{207,208}. By knocking out HPRT, 6TG will not be converted to 6GMP and the cell will become 6TG resistant²⁰⁷. This methodology has helped enrich genetically-modified hematopoietic progenitor and stem cells^{209, 210} but it was unclear if knocking down HPRT would impact T-cell function.

T-cells were previously observed to have loss of HPRT clinically, most prevalently in autoimmune cases, such as Lesch-Nyhan syndrome²¹¹⁻²¹⁵. Applicant hypothesized that using a short hairpin against HPRT would allow enrichment of CAR-T-cells by eliminating non-transduced T-cells through 6TG selection.

### Results

Optimizing shHPRT CAR Construct

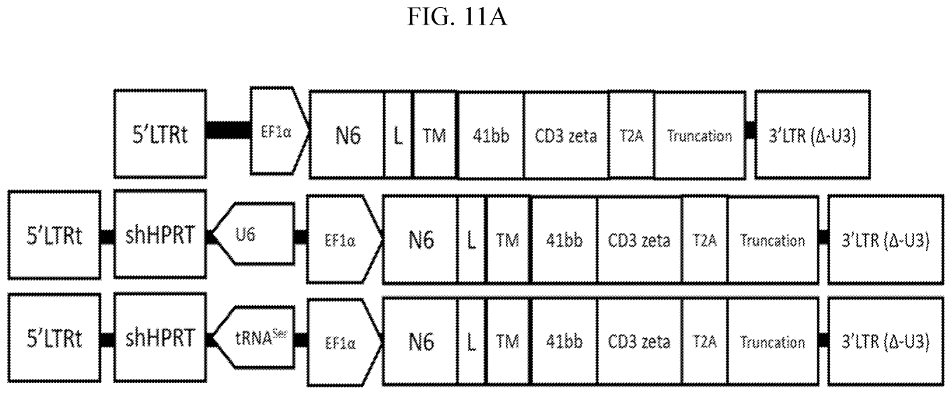

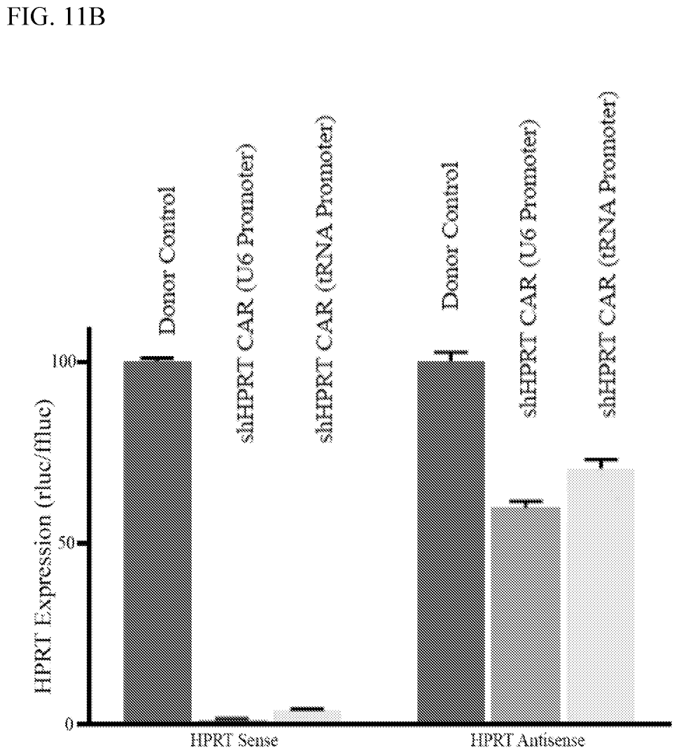

The shHPRT CAR, similar to shCCR5w shTat/Rev anti-HIV CAR, has a Pol III promoter to express the shRNA and was expressed in the antisense direction (FIG. 11A)¹⁸⁶. Since HPRT needs to be completely knocked down, the strong Pol III promoter (U6) was compared against a tRNA promoter (FIG. 11A). The tRNA promoter was beneficial for the protection anti-HIV CAR as the promoters have similar activity to U6 promoter but appear to have less off-target toxicity¹⁸⁶. Functionality of the construct was determined through dual luciferase knockdown assay. The HPRT target, both sense and antisense, were expressed in a Psi-check plasmid. The sense strand represents target knockdown and the antisense represents self-knockdown^{175, 186}. Both promoters had sufficient knockdown of the sense strand, while both had minimal effect on the antisense strand (FIG. 11B). Like shCCR5w, the shHPRT could be further optimized using wobble bases to remove the antisense targeting effect. It was noted that antisense targeting effects were not observed during lentiviral production^{188, 189}.

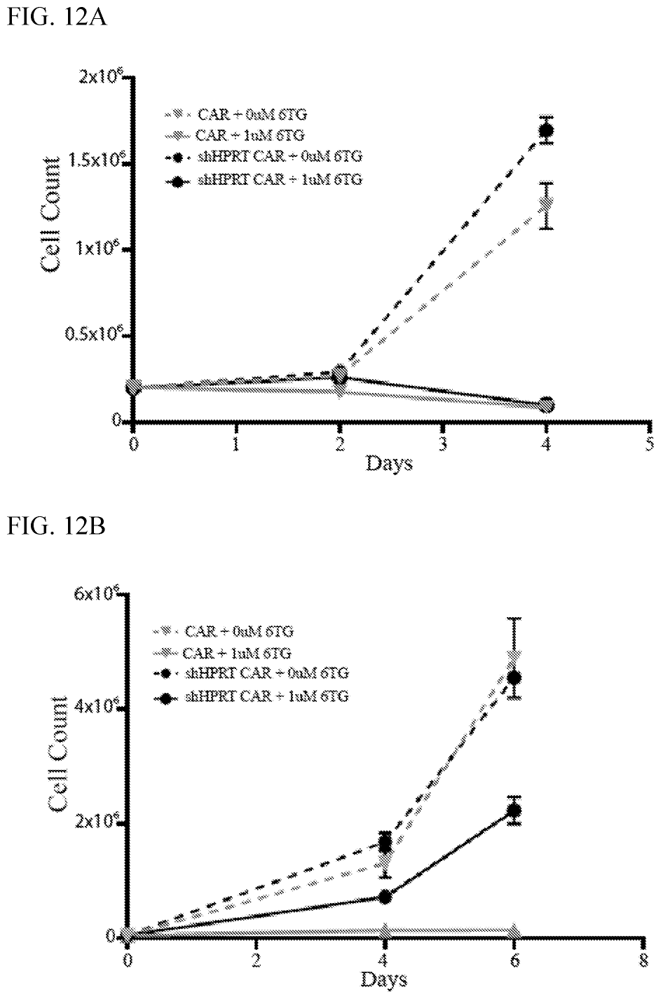

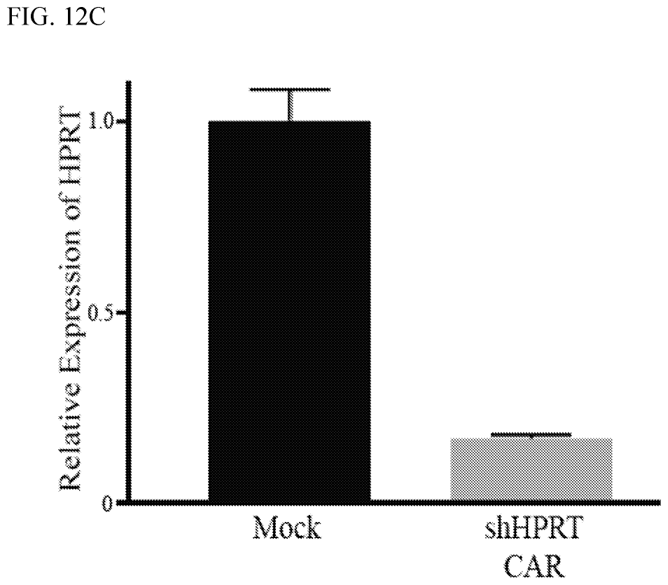

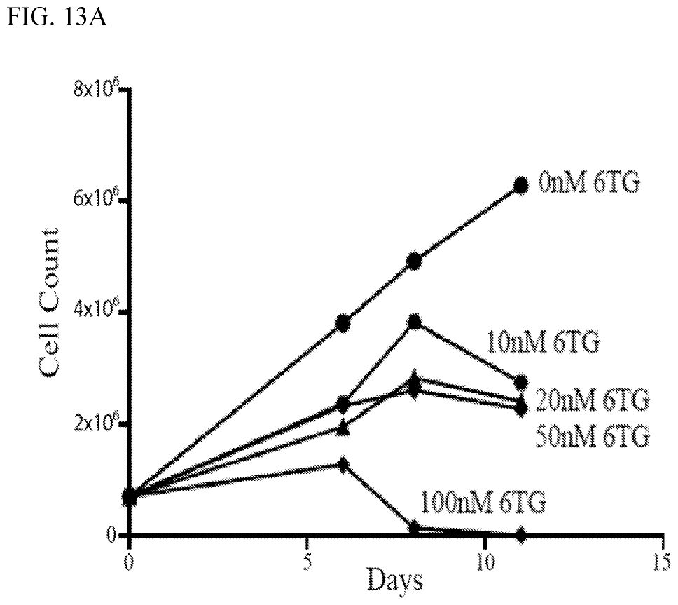

To test the enrichment potential of each construct, Jurkat cells were transduced with either U6 or tRNA promoter linked shHPRT anti-HIV CAR lentivirus and treated with either 0 or 1 μM 6TG. Conventional anti-HIV CAR (e.g. without shHPRT) was used as a control. The T-cells transduced with the construct including the tRNA promoter lacked enrichment following treatment with 6TG, which suggests that tRNA promoter was incapable of knocking down HPRT sufficiently to prevent 6TG processing (FIG. 12A). The T-cells transduced with the construct including the U6 promoter, on the other hand, had sufficient enrichment following administration of 6TG, suggesting optimal HPRT knockdown (FIG. 12B). This construct was therefore used for future studies.

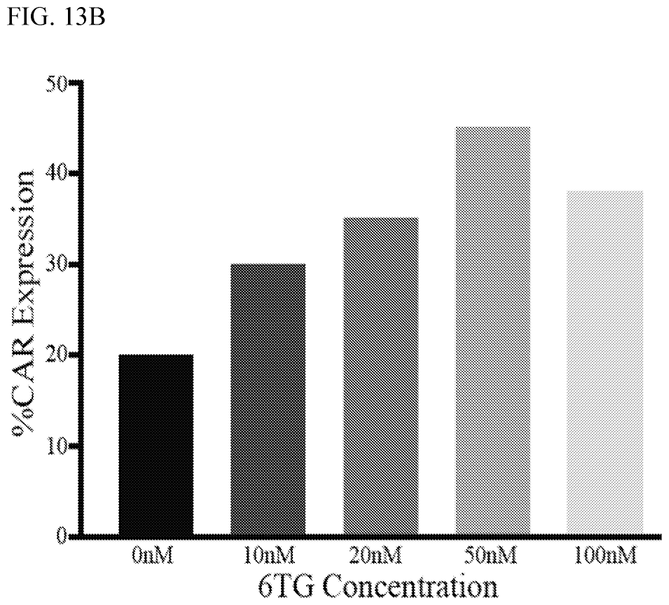

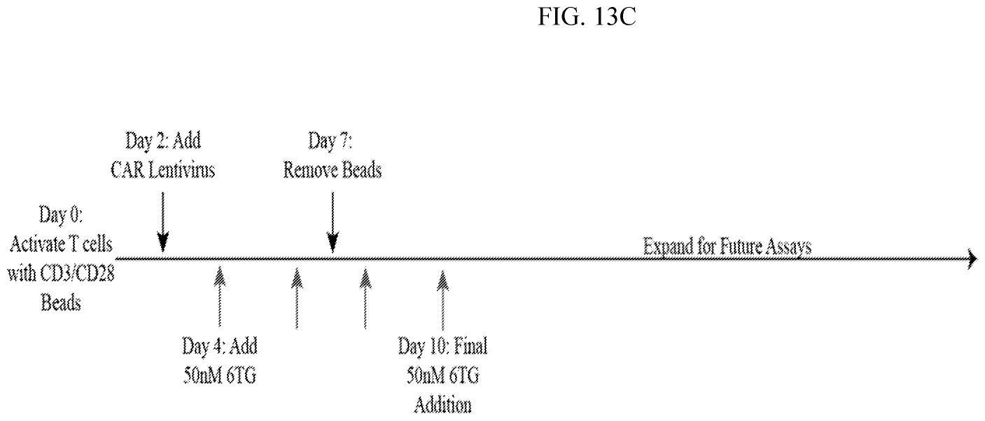

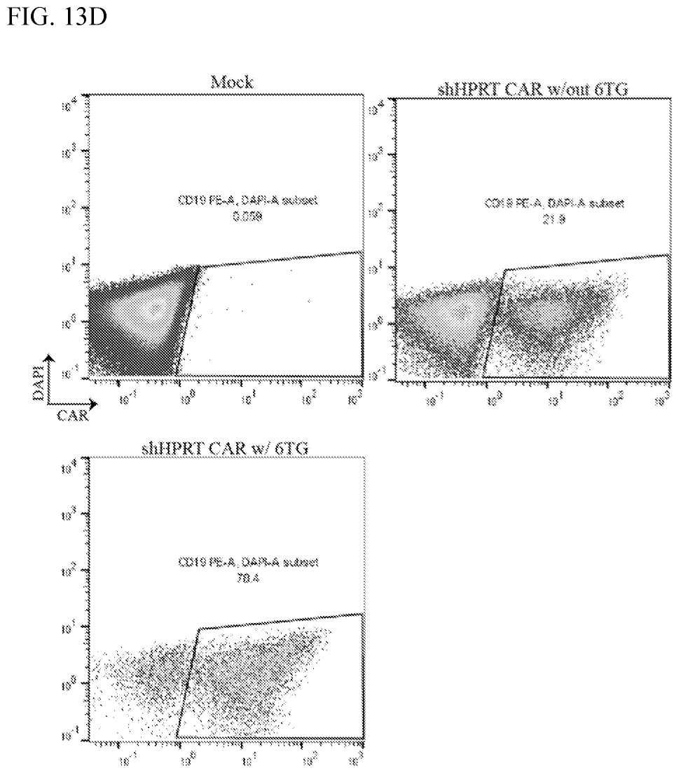

To test if an enrichment anti-HIV CAR construct (e.g. anti-HIV CAR construct including the shHPRT) would enrich for anti-HIV CAR T-cells from a population of T-cells, experiments were repeated by administering 1 M 6TG to the transduced Jurkat cells. In this experiment, a loss of all T-cells (data not shown), including those transduced with anti-HIV CAR construct, was observed, suggesting that the concentration of 6TG needed to be lowered. Applicant thus completed a dose titration to find the optimal dose of 6TG for enrichment CAR. Applicant treated the T-cells with various concentrations (10, 20, 50, and 100 nM) of 6TG. The cell counts of the T-cells, in addition to anti-HIV CAR expression percentage were measured over the course of culturing. It was observed that 10, 20, and 50 nM exerted toxic effects on the non-transduced T-cells, while 100 nM lead to a loss of a number of T-cells transduced with the enrichment anti-HIV CAR construct (FIG. 13A). It was shown that 50 nM of 6TG was observed to have the most enrichment potential for the anti-HIV CAR T-cells (FIG. 13B). However, there wasn't complete enrichment, which suggests that 6TG must be added several times over the course of culturing the cells. To address this, a multi-dosing regimen was designed that would allow addition of 6TG to the cell culture during the same time as cytokine addition (FIG. 13C). Following the four treatments of 6TG (n=3), there was selection of enrichment anti-HIV CAR T-cells from ˜20% anti-HIV CAR positivity to ˜80% anti-HIV CAR positivity (FIG. 13D).

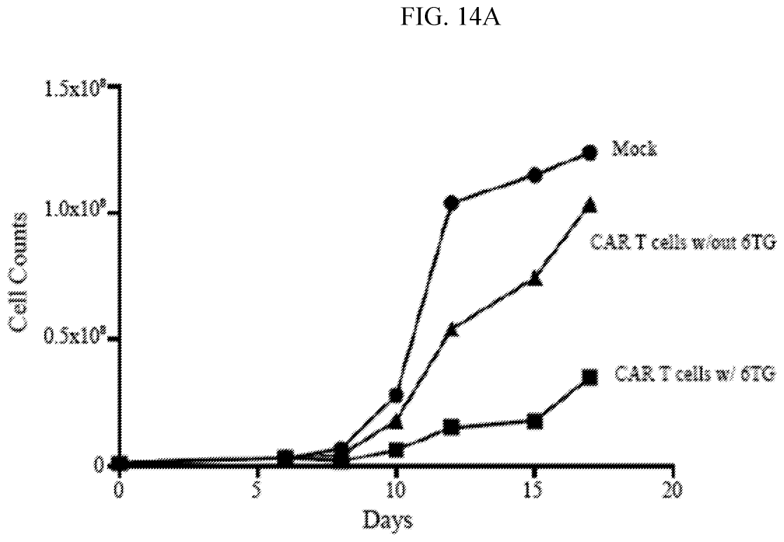

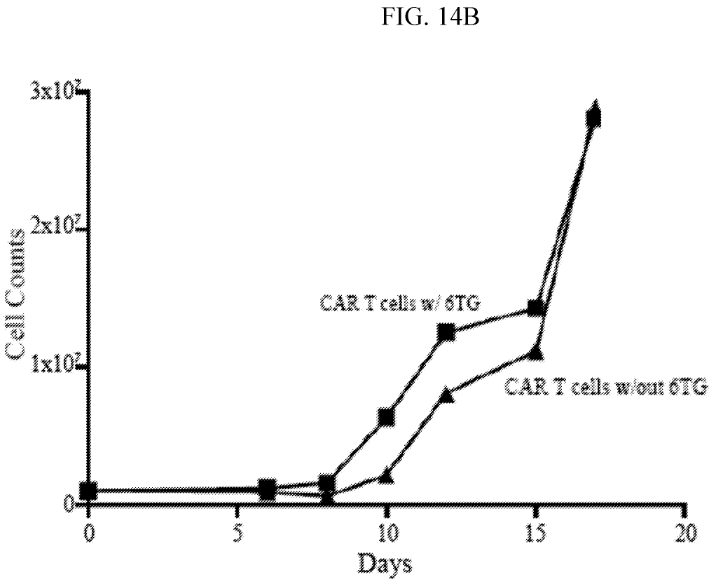

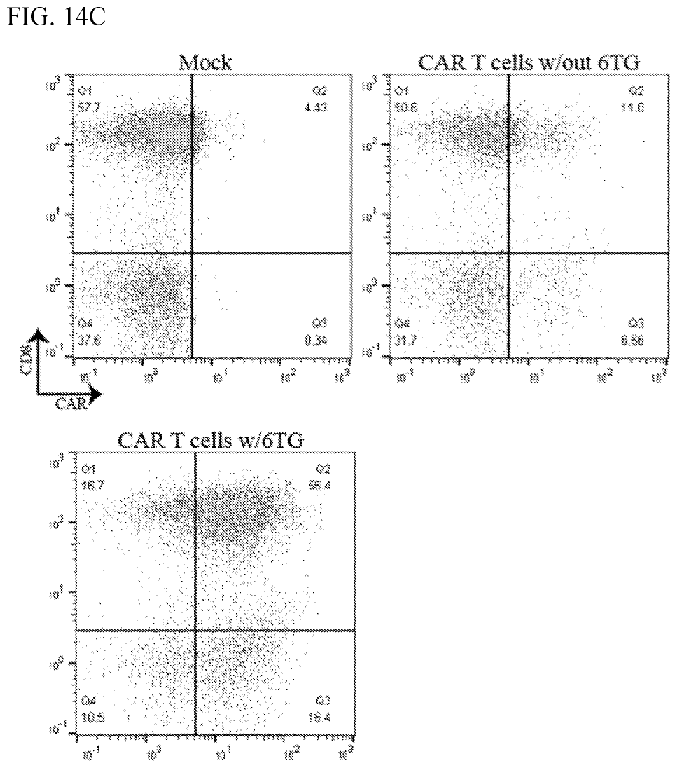

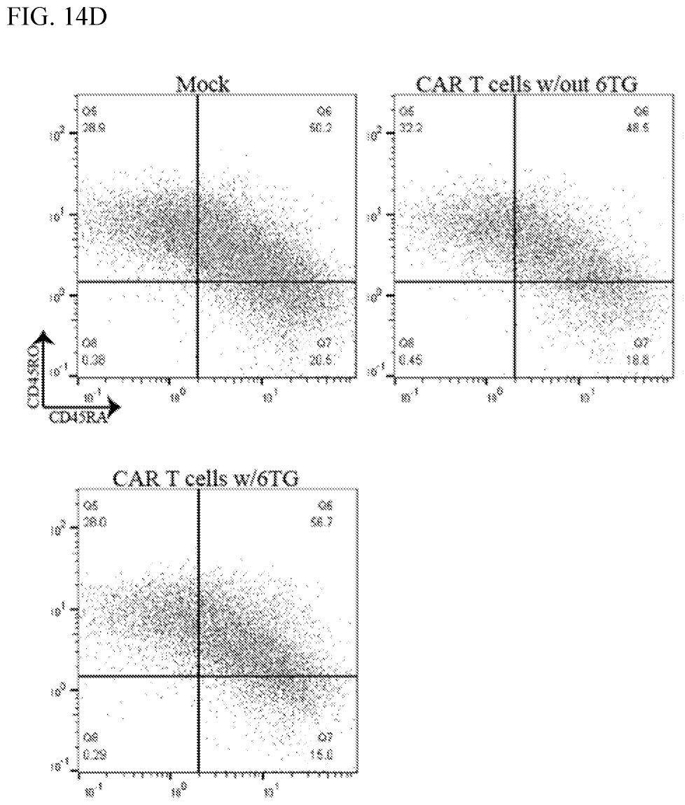

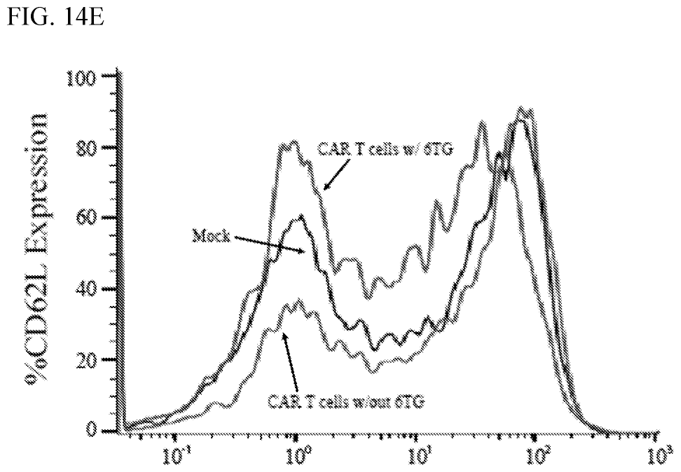

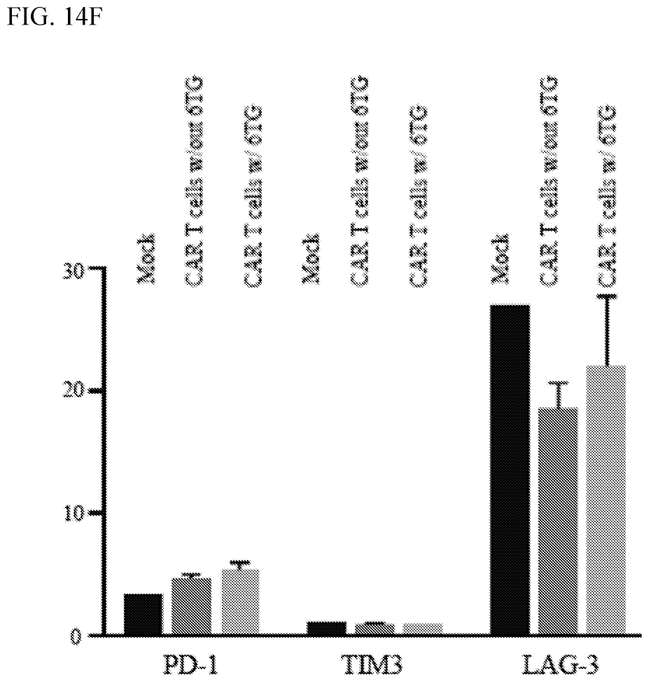

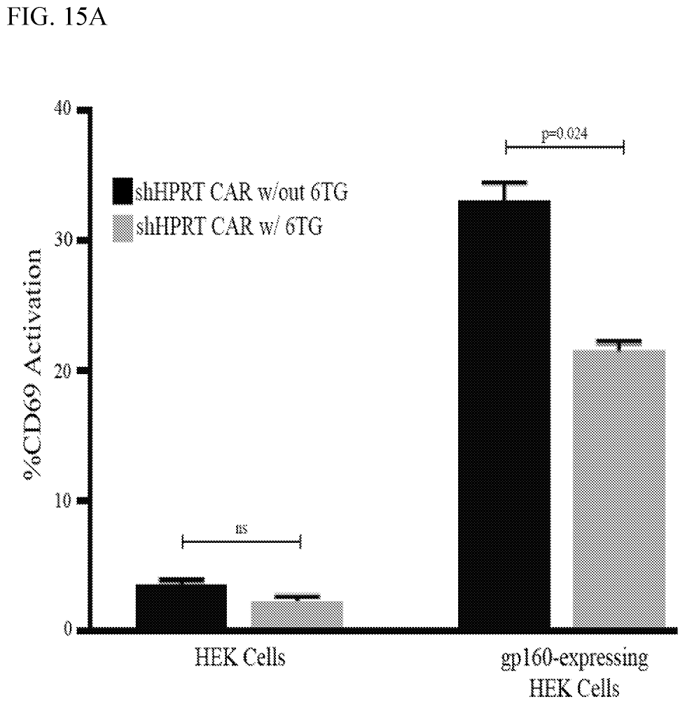

When analyzing the growth curve of the T-cells, Applicant observed that total T-cell growth decreased over the course of culture after treatment with 6TG (FIG. 14A). However, the growth of the 6TG-treated anti-HIV CAR T-cells growth was equivalent to anti-HIV CAR-T-cells that were not subjected to 6TG treatment (FIG. 14B).

Phenotypically, Applicant observed that the CD4 ratio dropped ˜10% following 6TG treatment (FIG. 14C), which could have potential consequences for CAR functionality¹⁵³. There was no observable difference between CD45RO and CD45RA ratios (FIG. 14D) but there was a noticeable decrease in CD62L ratios following 6TG selection (FIG. 14E). No noticeable changes in exhaustion makers were observed following 6TG treatment (FIG. 14F). While this data shows that the shIPRT allows for CAR T-cells enrichments with only minor phenotypic changes, it was necessary to determine if the enrichment process impacts anti-HIV CAR T-cell functionality.

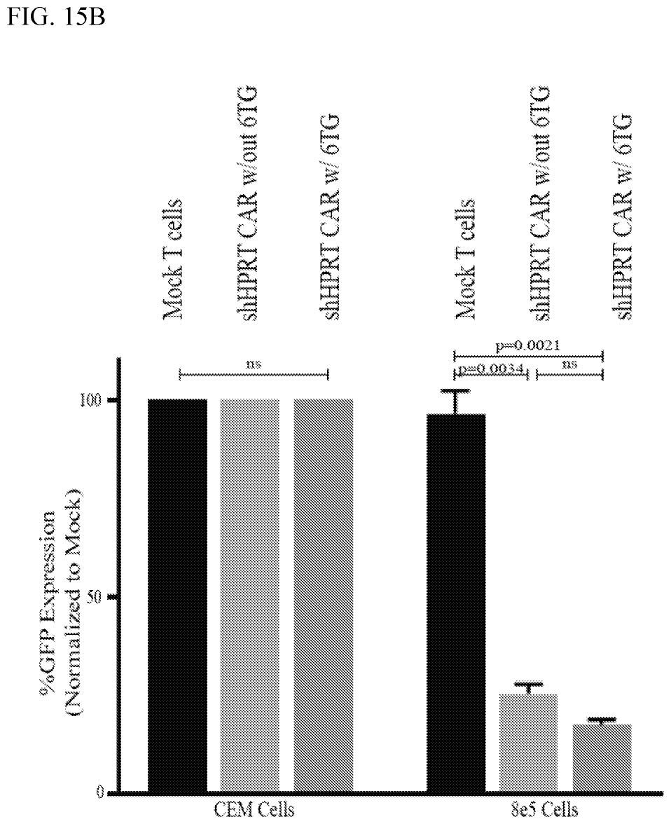

To test functionality, 6TG enriched anti-HIV CAR Jurkat cells were co-cultured with either gp160-expressing or parental HEK293 cells¹⁷⁶. Following a 24 hour incubation, CD69 expression was analyzed. Results showed that was a decrease in activation, as assessed by CD69 levels, following 6TG selection when compared to unselected anti-HIV CAR Jurkat cells (FIG. 15A). Lower activation could be beneficial for the anti-HIV CAR T-cells, since over-activation and anergy is a potential issue with anti-HIV CAR T-cell therapy^{193, 194}. Next, the cytotoxic functionality of 6TG enriched anti-HIV CAR T-cells (n=3) was tested. 6TG enriched or non-selected anti-HIV CAR T-cells were co-cultured with either 8e5.GFP or CEM.GFP cells. Applicant observed, when compared to mock-transduced T-cells, there was no difference in cytotoxicity for 6TG treated and non-treated enrichment anti-HIV CAR T-cells (FIG. 15B). This data shows that knocking down HPRT and selecting with 6TG does not impact anti-HIV CAR T-cell function.

### Conclusion

The studies presented herein shows that knocking out HPRT in CAR-T-cells allows for 6TG selection without impacting CAR T-cell function. Applicant confirmed that the strong Pol III promoter (U6) is more efficacious than the tRNA promoter. Applicant further optimized the dose of 6TG (50 nM) to enrich CAR T-cells over the course of production without impacting growth. A ˜10% difference in CD4/CD8 ratios was observed, as well as loss of CD62L expression when enriched; however, there was no observable difference in CD45RA/CD45RO ratio or exhaustion markers. Applicant also observed less activation in enriched shHPRT anti-HIV CAR Jurkat cells when compared to unenriched shHPRT anti-HIV CAR Jurkat cells. However, this lack of activation did not appear to affect cytotoxic efficacy when comparing enriched and unenriched shHPRT anti-HIV CAR T-cells. While this research was mainly focused on ex vivo enrichment of anti-HIV CAR T-cells, it is suggested that this could be expanded to a variety of anti-cancer CAR T-cells.

6TG has been used to treat hematological cancers²⁰⁶. Recent usage of 6TG has shown great response in maintenance therapy for children's acute lymphoblastic leukemia^{216, 217}. Although anti-CD19 CAR T-cells have shown a response rate of 80% in hematological malignancies, like acute lymphoblastic leukemia, the recent reports on relapse rates are roughly 50%²¹⁸. Applicant hypothesizes that combination of 6TG protection with anti-CD19 CAR T-cell therapy, may increase the percentage of patients that maintain remission.

### Example 4: Generation of 6-Mercaptopurine Resistant Anti-HIV CAR T-Cells

1E6 Jurkat cells were activated with Dynabeads™ human T-activator CD3/CD28+(Life Technologies). After 24 hours, the cells were transduced with shHPRT-expressing anti-HIV CAR (enrichment anti-HIV CAR) lentivirus at an MOI of 2. The cells were expanded in the presence of 50 U/mL recombinant human interleukin-2 (rhIL-2, Novartis) and 0.5 ng/mL interleukin-15 (rhIL-15, Novartis), which was supplemented every 48-72 hours for during the course of ex vivo expansion for 14-21 days (24).

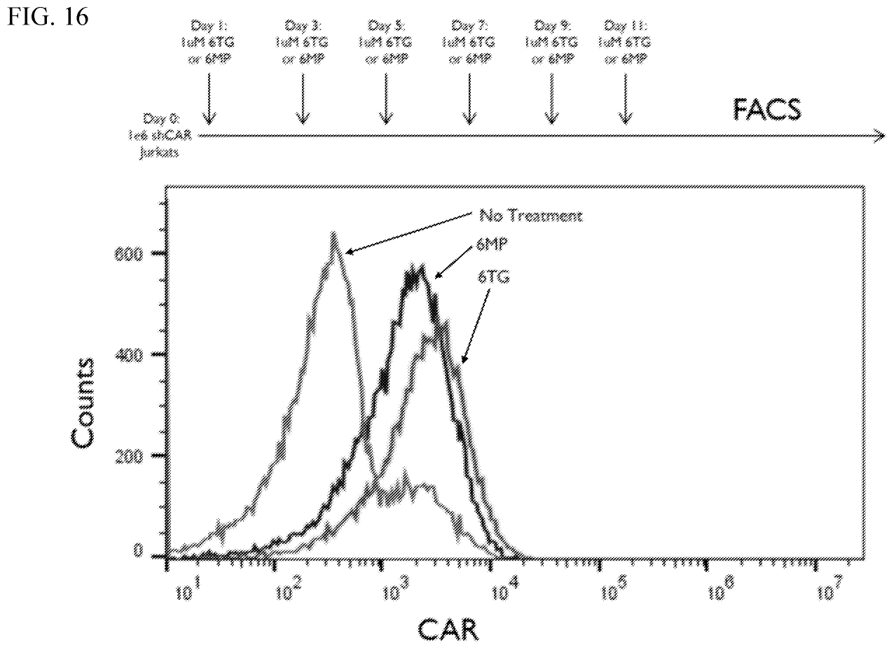

For selection, 1 uM 6-Thioguanine (6TG) or mercaptopurine (6-MP) was added every 48 hours starting on day 1 and ending on day 11. Untreated cells were used as control. The cells were then subject to FACs analysis for anti-HIV CAR expression (FIG. 16).

### REFERENCES

151. Maldini, C R, Gayout, K, Leibman, R S, Dopkin, D L, Mills, J P, Shan, X, et al. (2020). HIV-Resistant and HIV-Specific CAR-Modified CD4(+) T Cells Mitigate HIV Disease Progression and Confer CD4(+) T Cell Help In Vivo. Mol Ther 28: 1585-1599.
152. Maldini, C R, Claiborne, D T, Okawa, K, Chen, T, Dopkin, D L, Shan, X, et al. (2020). Dual CD4-based CAR T cells with distinct costimulatory domains mitigate HIV pathogenesis in vivo. Nat Med.
153. Wang, D, Aguilar, B, Starr, R, Alizadeh, D, Brito, A, Sarkissian, A, et al. (2018). Glioblastoma-targeted CD4+ CAR T cells mediate superior antitumor activity. JCI insight 3: e99048.
154. Cannon, P, and June, C (2011). Chemokine receptor 5 knockout strategies. Current opinion in HIV and AIDS 6: 74-79.
155. Anthony-Gonda, K, Bardhi, A, Ray, A, Flerin, N, Li, M, Chen, W, et al. (2019). Multispecific anti-HIV duoCAR-T cells display broad in vitro antiviral activity and potent in vivo elimination of HIV-infected cells in a humanized mouse model. Science Translational Medicine 11: eaav5685.
156. Di Mascio, M, Dornadula, G, Zhang, H, Sullivan, J, Xu, Y, Kulkosky, J, et al. (2003). In a subset of subjects on highly active antiretroviral therapy, human immunodeficiency virus type 1 RNA in plasma decays from 50 to <5 copies per milliliter, with a half-life of 6 months. Journal of virology 77: 2271-2275.
157. Chattopadhyay, S K, Morse, H C, 3rd, Makino, M, Ruscetti, S K, and Hartley, J W (1989). Defective virus is associated with induction of murine retrovirus-induced immunodeficiency syndrome. Proc Natl Acad Sci USA 86: 3862-3866.
158. Voynow, S L, and Coffin, J M (1985). Truncated gag-related proteins are produced by large deletion mutants of Rous sarcoma virus and form virus particles. Journal of virology 55: 79-85.
159. Holland, J J, and Villarreal, LP (1975). Purification of defective interfering T particles of vesicular stomatitis and rabies viruses generated in vivo in brains of newborn mice. Virology 67: 438-449.
160. Aaskov, J, Buzacott, K, Thu, H M, Lowry, K, and Holmes, E C (2006). Long-term transmission of defective RNA viruses in humans and Aedes mosquitoes. Science 311: 236-238.
161. Li, D, Lott, W B, Lowry, K, Jones, A, Thu, H M, and Aaskov, J (2011). Defective interfering viral particles in acute dengue infections. PLoS ONE 6: e19447.
162. Tanner, E J, Jung, S-Y, Glazier, J, Thompson, C, Zhou, Y, Martin, B, et al. (2019). Discovery and Engineering of a Therapeutic Interfering Particle (TIP): a combination self-renewing antiviral. bioRxiv: 820456.
163. Metzger, V T, Lloyd-Smith, J O, and Weinberger, L S (2011). Autonomous targeting of infectious superspreaders using engineered transmissible therapies. PLoS computational biology 7: e1002015.
164. Rast, L I, Rouzine, I M, Rozhnova, G, Bishop, L, Weinberger, A D, and Weinberger, L S (2016). Conflicting Selection Pressures Will Constrain Viral Escape from Interfering Particles: Principles for Designing Resistance-Proof Antivirals. PLoS computational biology 12: e1004799.
165. Rouzine, I M, and Weinberger, L S (2013). Design requirements for interfering particles to maintain coadaptive stability with HIV-1. Journal of virology 87: 2081-2093.
166. Shrivastava, S, Charlins, P, Ackley, A, Embree, H, Dropulic, B, Akkina, R, et al. (2018). Stable Transcriptional Repression and Parasitism of HIV-1. Molecular therapy Nucleic acids 12: 12-18.
167. Levine, B L, Humeau, L M, Boyer, J, MacGregor, R R, Rebello, T, Lu, X, et al. (2006). Gene transfer in humans using a conditionally replicating lentiviral vector. Proc Natl Acad Sci USA 103: 17372-17377.
168. Mukherjee, R, Plesa, G, Sherrill-Mix, S, Richardson, M W, Riley, J L, and Bushman, F D (2010). HIV sequence variation associated with env antisense adoptive T-cell therapy in the hNSG mouse model. Mol Ther 18: 803-811.
169. Lu, X, Yu, Q, Binder, G K, Chen, Z, Slepushkina, T, Rossi, J, et al. (2004). Antisense-mediated inhibition of human immunodeficiency virus (HIV) replication by use of an HIV type 1-based vector results in severely attenuated mutants incapable of developing resistance. J Virol 78: 7079-7088.
170. Reyes-Darias, J A, Sanchez-Luque, F J, and Berzal-Herranz, A (2008). Inhibition of HIV-1 replication by RNA-based strategies. Current HIV research 6: 500-514.
171. Rossi, J J, June, C H, and Kohn, D B (2007). Genetic therapies against HIV. Nat Biotechnol 25: 1444-1454.
172. Scherer, L, Rossi, J J, and Weinberg, M S (2007). Progress and prospects: RNA-based therapies for treatment of HIV infection. Gene Ther 14: 1057-1064.
173. Wang, G P, Levine, B L, Binder, G K, Berry, C C, Malani, N, McGarrity, G, et al. (2009). Analysis of lentiviral vector integration in HIV+ study subjects receiving autologous infusions of gene modified CD4+ T cells. Molecular therapy 17: 844-850.
174. Dropulid, B, Herminkovi, M, and Pitha, P M (1996). A conditionally replicating HIV-1 vector interferes with wild-type HIV-1 replication and spread. Proceedings of the National Academy of Sciences 93: 11103-11108.
175. Kumar, M, Keller, B, Makalou, N, and Sutton, R E (2001). Systematic determination of the packaging limit of lentiviral vectors. Human gene therapy 12: 1893-1905.
176. Chen, J, Kovacs, J M, Peng, H, Rits-Volloch, S, Lu, J, Park, D, et al. (2015). HIV-1 ENVELOPE. Effect of the cytoplasmic domain on antigenic characteristics of HIV-1 envelope glycoprotein. Science (New York, NY) 349: 191-195.
177. Wilburn, K M, Mwandumba, H C, Jambo, K C, Boliar, S, Solouki, S, Russell, D G, et al. (2016). Heterogeneous loss of HIV transcription and proviral DNA from 8E5/LAV lymphoblastic leukemia cells revealed by RNA FISH:FLOW analyses. Retrovirology 13: 55.
178. An, D S, Donahue, R E, Kamata, M, Poon, B, Metzger, M, Mao, S-H, et al. (2007). Stable reduction of CCR5 by RNAi through hematopoietic stem cell transplant in non-human primates. Proceedings of the National Academy of Sciences 104: 13110-13115.
179. Shimizu, S, Hong, P, Arumugam, B, Pokomo, L, Boyer, J, Koizumi, N, et al. (2010). A highly efficient short hairpin RNA potently down-regulates CCR5 expression in systemic lymphoid organs in the hu-BLT mouse model. Blood, The Journal of the American Society of Hematology 115: 1534-1544.
180. Ringpis, G-EE, Shimizu, S, Arokium, H, Camba-Colón, J, Carroll, MV, Cortado, R, et al. (2013). Engineering HIV-1-Resistant T-Cells from Short-Hairpin RNA-Expressing Hematopoietic Stem/Progenitor Cells in Humanized BLT Mice. PLoS ONE 7: e53492.
181. Scarborough, R J, and Gatignol, A (2017). RNA Interference Therapies for an HIV-1 Functional Cure. Viruses 10: 8.
182. Aagaard, L A, Zhang, J, von Eije, K J, Li, H, Saetrom, P, Amarzguioui, M, et al. (2008). Engineering and optimization of the miR-106b cluster for ectopic expression of multiplexed anti-HIV RNAs. Gene therapy 15: 1536-1549.
183. Pang, K M, Castanotto, D, Li, H, Scherer, L, and Rossi, J J (2018). Incorporation of aptamers in the terminal loop of shRNAs yields an effective and novel combinatorial targeting strategy. Nucleic Acids Res 46: e6.
184. DiGiusto, D L, Krishnan, A, Li, L, Li, H, Li, S, Rao, A, et al. (2010). RNA-based gene therapy for HIV with lentiviral vector-modified CD34(+) cells in patients undergoing transplantation for AIDS-related lymphoma. Science translational medicine 2: 36ra43-36ra43.
185. Li, M-J, Kim, J, Li, S, Zaia, J, Yee, J-K, Anderson, J, et al. (2005). Long-Term Inhibition of HIV-1 Infection in Primary Hematopoietic Cells by Lentiviral Vector Delivery of a Triple Combination of Anti-HIV shRNA, Anti-CCR5 Ribozyme, and a Nucleolar-Localizing TAR Decoy. Molecular Therapy 12: 900-909.
186. Scherer, L J, Frank, R, and Rossi, J J (2007). Optimization and characterization of tRNA-shRNA expression constructs. Nucleic acids research 35: 2620-2628.
187. Liu, Y P, Vink, M A, Westerink, J-T, Ramirez de Arellano, E, Konstantinova, P, Ter Brake, O, et al. (2010). Titers of lentiviral vectors encoding shRNAs and miRNAs are reduced by different mechanisms that require distinct repair strategies. RNA (New York, NY) 16: 1328-1339.
188. Brake, Ot, Hooft, Kt, Liu, Y P, Centlivre, M, Jasmijn von Eije, K, and Berkhout, B (2008). Lentiviral Vector Design for Multiple shRNA Expression and Durable HIV-1 Inhibition. Molecular Therapy 16: 557-564.
189. Massengill, M T, Young, B M, Lewin, A S, and Ildefonso, C J (2019). Co-Delivery of a Short-Hairpin RNA and a shRNA-Resistant Replacement Gene with Adeno-Associated Virus: An Allele-Independent Strategy for Autosomal-Dominant Retinal Disorders. Methods in molecular biology (Clifton, NJ) 1937: 235-258.
190. Cibriin, D, and Sanchez-Madrid, F (2017). CD69: from activation marker to metabolic gatekeeper. Eur J Immunol 47: 946-953.
191. Glorieux, C, and Huang, P (2019). CD137 expression in cancer cells: regulation and significance. Cancer Commun (Lond) 39: 70-70.
192. Abramson, J S, Irwin, K E, Frigault, M J, Dietrich, J, McGree, B, Jordan, J T, et al. (2019). Successful anti-CD19 CAR T-cell therapy in HIV-infected patients with refractory high-grade B-cell lymphoma. Cancer 125: 3692-3698.
193. Ishii, K, Pouzolles, M, Chien, C D, Erwin-Cohen, R A, Kohler, M E, Qin, H, et al. (2020). Perforin-deficient CAR T cells recapitulate late-onset inflammatory toxicities observed in patients. The Journal of Clinical Investigation 130: 5425-5443.
194. Yang, Y, Kohler, M E, Chien, C D, Sauter, C T, Jacoby, E, Yan, C, et al. (2017). TCR engagement negatively affects CD8 but not CD4 CAR T cell expansion and leukemic clearance. Science Translational Medicine 9: eaag1209.
195. Casucci, M, Falcone, L, Camisa, B, Norelli, M, Porcellini, S, Stornaiuolo, A, et al. (2018). Extracellular NGFR Spacers Allow Efficient Tracking and Enrichment of Fully Functional CAR-T Cells Co-Expressing a Suicide Gene. Frontiers in Immunology 9.
196. Berger, C, Flowers, M E, Warren, E H, and Riddell, S R (2006). Analysis of transgene-specific immune responses that limit the in vivo persistence of adoptively transferred HSV-TK-modified donor T cells after allogeneic hematopoietic cell transplantation. Blood 107: 2294-2302.
197. Jonnalagadda, M, Brown, C E, Chang, W-C, Ostberg, J R, Forman, S J, and Jensen, M C (2013). Engineering Human T Cells for Resistance to Methotrexate and Mycophenolate Mofetil as an In Vivo Cell Selection Strategy. PLoS ONE 8: e65519.
198. Milsom, M D, and Fairbairn, L J (2004). Protection and selection for gene therapy in the hematopoietic system. The Journal of Gene Medicine: A cross-disciplinary journal for research on the science of gene transfer and its clinical applications 6: 133-146.
199. Muul, L M, and Candotti, F (2007). Immune responses to gene-modified T cells. Current gene therapy 7: 361-368.
200. Riddell, S R, Elliott, M, Lewinsohn, D A, Gilbert, M J, Wilson, L, Manley, S A, et al. (1996). T-cell mediated rejection of gene-modified HIV-specific cytotoxic T lymphocytes in HIV-infected patients. Nature medicine 2: 216-223.
201. Volpato, J P, Mayotte, N, Fossati, E, Guerrero, V, Sauvageau, G, and Pelletier, J N (2011). Selectively weakened binding of methotrexate by human dihydrofolate reductase allows rapid ex vivo selection of mammalian cells. Journal of Molecular Recognition 24: 188-198.
202. Walker, R E, Carter, C S, Muul, L, Natarajan, V, Herpin, B R, Leitman, S F, et al. (1998). Peripheral expansion of pre-existing mature T cells is an important means of CD4+ T-cell regeneration HIV-infected adults. Nature medicine 4: 852-856.
203. Allison, A C, and Eugui, E M (2000). Mycophenolate mofetil and its mechanisms of action. Immunopharmacology 47: 85-118.
204. Ercikan-Abali, E A, Mineishi, S, Tong, Y, Nakahara, S, Waltham, MC, Banerjee, D, et al. (1996). Active site-directed double mutants of dihydrofolate reductase. Cancer research 56: 4142-4145.
205. Shimmura, H, Tanabe, K, Habiro, K, Abe, R, and Toma, H (2006). Combination effect of mycophenolate mofetil with mizoribine on cell proliferation assays and in a mouse heart transplantation model. Transplantation 82: 175-179.
206. Munshi, P N, Lubin, M, and Bertino, J R (2014). 6-thioguanine: a drug with unrealized potential for cancer therapy. Oncologist 19: 760-765.
207. Aubrecht, J, Goad, M E, and Schiestl, R H (1997). Tissue specific toxicities of the anticancer drug 6-thioguanine is dependent on the Hprt status in transgenic mice. Journal of Pharmacology and Experimental Therapeutics 282: 1102-1108.
208. Coulthard, S, and Hogarth, L (2005). The thiopurines: an update. Investigational new drugs 23: 523-532.
209. Choudhary, R, Baturin, D, Fosmire, S, Freed, B, and Porter, CC (2013). Knockdown of HPRT for Selection of Genetically Modified Human Hematopoietic Progenitor Cells. PLoS ONE 8: e59594.
210. Hacke, K, Szakmary, A, Cuddihy, A R, Rozengurt, N, Lemp, N A, Aubrecht, J, et al. (2012). Combined preconditioning and in vivo chemoselection with 6-thioguanine alone achieves highly efficient reconstitution of normal hematopoiesis with HPRT-deficient bone marrow. Experimental hematology 40: 3-13. e13.
211. Torres, R J, and Puig, J G (2007). Hypoxanthine-guanine phosophoribosyltransferase (HPRT) deficiency: Lesch-Nyhan syndrome. Orphanet journal of rare diseases 2: 48-48.
212. Finette, B A, Poseno, T, and Albertini, RJ (1996). V (D) J recombinase-mediated HPRT mutations in peripheral blood lymphocytes of normal children. Cancer research 56: 1405-1412.
213. Fuscoe, J C, Zimmerman, L J, Lippert, M J, Nicklas, J A, O'Neill, J P, and Albertini, R J (1991). V (D) J recombinase-like activity mediates hprt gene deletion in human fetal T-lymphocytes. Cancer research 51: 6001-6005.
214. Murray, J M, Messier, T, Rivers, J, O'Neill, J P, Walker, V E, Vacek, P M, et al. (2012). V(D)J Recombinase-Mediated TCR R Locus Gene Usage and Coding Joint Processing in Peripheral T Cells during Perinatal and Pediatric Development. The Journal of Immunology 189: 2356-2364.
215. Murray, J M, O'Neill, J P, Messier, T, Rivers, J, Walker, V E, McGonagle, B, et al. (2006). V (D) J recombinase-mediated processing of coding junctions at cryptic recombination signal sequences in peripheral T cells during human development. The Journal of Immunology 177: 5393-5404.
216. Chen, L, Yan, H-X, Liu, X-W, and Chen, W-X (2020). Clinical efficacy and safety of 6-thioguanine in the treatment of childhood acute lymphoblastic leukemia: A protocol for systematic review and meta-analysis. Medicine 99: e20082-e20082.
217. Heath, J L, Aumann, W K, Maxfield, C M, and Wechsler, D S (2018). Isolated central nervous system chloroma as a presenting sign of relapsed pediatric acute lymphoblastic leukemia. Journal of Pediatric Hematology Oncology 40: e442-e445.

INFORMAL SEQUENCE LISTING

SEQ ID NO: 1 (DNA for anti-Tat/Rev shRNA)
GCGGAGACAGCGACGAAGAGCTTTGTGTAGGCTCTTCGTCGCTGTCTCCGC
SEQ ID NO: 2 (DNA for anti-CCR5 shRNA wobble base (shCCR5w))
AGTGTCAAGTCCAATTTGTGGTTCAAGAGATCATAGATTGGACTTGACACTTT
SEQ ID NO: 3 (DNA for anti-HPRT shRNA)
GCACTGAATAGAAATGGTGGTTCAAGAGATCACTATTTCTATTCAGTGC
SEQ ID NO: 4 (N6 antibody VH)
RAHLVQSGTAMKKPGASVRVSCQTSGYTFTAHILFWFRQAPGRGLEWVGWIKPQY
GAVNFGGGFRDRVTLTRDVYREIAYMDIRGLKPDDTAVYYCARDRSYGDSSWALD
AWGQGTTVVVSA
SEQ ID NO: 5 (N6 antibody VL)
YIHVTQSPSSLSVSIGDRVTINCQTSQGVGSDLHWYQHKPGRAPKLLIHHTSSVEDGV
PSRFSGSGFHTSFNLTISDLQADDIATYYCQVLQFFGRGSRLHIK
SEQ ID NO: 6 (NIH-45-46 antibody VH)
QVRLSQSGGQMKKPGESMRLSCRASGYEFLNCPINWIRLAPGRRPEWMGWLKPRG
GAVNYARKFQGRVTMTRDVYSDTAFLELRSLTSDDTAVYFCTRGKYCTARDYYNW
DFEHWGRGAPVTVSS
SEQ ID NO: 7 (NIH-45-46 antibody VL)
EIVLTQSPATLSLSPGETAIISCRTSQSGSLAWYQQRPGQAPRLVIYSGSTRAAGIPDRF
SGSRWGADYNLSISNLESGDFGVYYCQQYEFFGQGTKVQVDIK
SEQ ID NO: 8 (DNA for anti-CCR5 shRNA-Original)
AGTGTCAAGTCCAATCTATGATTCAAGAGATCATAGATTGGACTTGACACTTT
SEQ ID NO: 9 (anti-CCR5 shRNA-Original)
AGUGUCAAGUCCAAUCUAUGAUUCAAGAGAUCAUAGAUUGGACUUGACACUU
U
SEQ ID NO: 10 (anti-Tat/Rev shRNA)
GCGGAGACAGCGACGAAGAGCUUUGUGUAGGCUCUUCGUCGCUGUCUCCGCUU
SEQ ID NO: 11 (anti-CCR5 shRNA wobble base (shCCR5w))
AGUGUCAAGUCCAAUUUGUGGUUCAAGAGAUCAUAGAUUGGACUUGACACUU
U
SEQ ID NO: 12 (anti-HPRT shRNA)
GCACUGAAUAGAAAUGGUGGUUCAAGAGAUCACUAUUUCUAUUCAGUGCUU
SEQ ID NO: 13 (PGT121 antibody VL)
SDISVAPGETARISCGEKSLGSRAVQWYQHRAGQAPSLIIYNNQDRPSGIPERFSGSPD
SPFGTTATLTITSVEAGDEADYYCHIWDSRVPTKWVFGGGTTLTV
SEQ ID NO: 14 (PGT121 antibody VH)
QMQLQESGPGLVKPSETLSLTCSVSGASISDSYWSWIRRSPGKGLEWIGYVHKSGDT
NYSPSLKSRVNLSLDTSKNQVSLSLVAATAADSGKYYCARTLHGRRIYGIVAFNEWF
TYFYMDVWGNGTQVTVSS
SEQ ID NO: 15 (PGT128 antibody VL)
QSALTQPPSASGSPGQSITISCTGTSNNFVSWYQQHAGKAPKLVIYDVNKRPSGVPDR
FSGSKSGNTASLTVSGLQTDDEAVYYCGSLVGNWDVIFGGGTKLTVL
SEQ ID NO: 16 (PGT128 antibody VH)
QPQLQESGPTLVEASETLSLTCAVSGDSTAACNSFWGWVRQPPGKGLEWVGSLSHC
ASYWNRGWTYHNPSLKSRLTLALDTPKNLVFLKLNSVTAADTATYYCARFGGEVL
RYTDWPKPAWVDLWGRGTLVTVSS
SEQ ID NO: 17 (3BC176 antibody VL)
QSVLTQPASVSASPGQSITVSCTGSRNDVGGYDFVSWYQRHPGGVPKLIIYEISKRPS
GIPQRFSGSRSGNTASLTISGLQDDDEADYYCCSYASYDRLIFGGGTRVSVL
SEQ ID NO: 18 (3BC176 antibody VH)
QVQLMQSGAQLRDPGDSLKISCKASGYNFIDYHIHWVRLAPGRGLEWMGWIDPVG
GITKYAGQFQGRLSLTRDTSTNTLFLELSRLTAGDTAVYFCARSMRPVDHGIDYSGL
FVFHFWGRGSDVLVSS

1. A T-cell comprising:
a nucleic acid comprising a sequence encoding an anti-HIV Chimeric Antigen Receptor (CAR),
a sequence encoding a Tat/Rev inhibitor and
a sequence encoding a CCR5 inhibitor.

2. (canceled)

3. The T-cell of claim 1, wherein said Tat/Rev inhibitor is an anti-Tat/Rev shRNA.

4. The T-cell of claim 3, wherein the anti-Tat/Rev shRNA comprises a sequence having at least 95% sequence identity to SEQ ID NO:10.

5. The T-cell of claim 1, wherein said CCR5 inhibitor is an anti-CCR5 shRNA.

6. The T-cell of claim 5, wherein said anti-CCR5 shRNA comprises a sequence having at least 95% sequence identity to SEQ ID NO:9 or 11.

7. The T-cell of claim 1, wherein said nucleic acid further comprises a sequence encoding a hypoxanthine phosphoribosyl transferase (HPRT) inhibitor.

8. (canceled)

9. (canceled)

10. The T-cell of claim 5, wherein the anti-CCR5 shRNA comprises a wobble base.

11. The T-cell of claim 1, wherein the anti-HIV CAR comprises:
(i) an antibody region capable of binding an HIV envelope protein; and
(ii) a transmembrane domain.

12. The T-cell of claim 11, wherein the HIV envelope protein is gp120 or gp41.

13. The T-cell of claim 11, wherein the antibody region comprises a heavy chain variable domain comprising the sequence of SEQ ID NO:4 and a light chain variable domain comprising the sequence of SEQ ID NO:5.

14. The T-cell of claim 11, wherein the antibody region comprises a heavy chain variable domain comprising the sequence of SEQ ID NO:6 and a light chain variable domain comprising the sequence of SEQ ID NO:7.

15.-28. (canceled)

29. A pharmaceutical composition comprising the T-cell of claim 1 and a pharmaceutically acceptable excipient.

30. A nucleic acid comprising a sequence encoding an anti-HIV Chimeric Antigen Receptor (CAR),
a sequence encoding a Tat/Rev inhibitor and
a sequence encoding a CCR5 inhibitor.

31. (canceled)

32. The nucleic acid of claim 30, further comprising a sequence encoding a hypoxanthine phosphoribosyl transferase (HPRT) inhibitor.

33.-35. (canceled)

36. A method of treating an HIV-infected subject in need thereof, said method comprising administering to said subject an effective amount of the T-cell of claim 1.

37.-39. (canceled)

40. A method of treating an HIV-infected subject in need thereof, said method comprising:
(i) contacting a population of T-cells with the nucleic acid of claim 32, thereby forming a population of transduced and non-transduced T-cells;
(ii) contacting the population of transduced and non-transduced T-cells with an amount of 6-thioguanine (6TG) or 6-mercaptopurine (6-MP) effective to cause cell death in the non-transduced T-cells, thereby forming a population of transduced T-cells; and
(iii) administering to said subject an effective amount of said population of transduced T-cells.

41.-56. (canceled)

57. A method of selecting a population of transduced T-cells comprising:
(i) contacting a population of T-cells with the nucleic acid of claim 32, thereby forming a population of transduced and non-transduced T-cells; and
(ii) contacting the population of transduced and non-transduced T-cells with an amount of 6-thioguanine (6TG) or 6-mercaptopurine (6-MP) effective to cause cell death in the non-transduced T-cells, thereby selecting the population of transduced T-cells.

58.-71. (canceled)

72. The T-cell of claim 11, wherein the antibody region is a single-chain variable fragment (scFv).

73. The T-cell of claim 11, wherein the antibody region comprises the CDR sequences of N6.

74. The T-cell of claim 11, wherein the antibody region comprises the CDR sequences of NIH-45-46.


SEQUENCE LISTING (18 sequences)

SEQ ID NO: 1 - DNA, 51 bp, Artificial Sequence
  Synthetic Construct
gcggagacagcgacgaagagctttgtgtaggctcttcgtcgctgtctccgc

SEQ ID NO: 2 - DNA, 53 bp, Artificial Sequence
  Synthetic Construct
agtgtcaagtccaatttgtggttcaagagatcatagattggacttgacacttt

SEQ ID NO: 3 - DNA, 49 bp, Artificial Sequence
  Synthetic Construct
gcactgaatagaaatggtggttcaagagatcactatttctattcagtgc

SEQ ID NO: 4 - protein, 122 aa, Artificial Sequence [sequence omitted]

SEQ ID NO: 5 - protein, 103 aa, Artificial Sequence [sequence omitted]

SEQ ID NO: 6 - protein, 125 aa, Artificial Sequence
  Synthetic Construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSS

SEQ ID NO: 7 - protein, 103 aa, Artificial Sequence
  Synthetic Construct
EIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIK

SEQ ID NO: 8 - DNA, 53 bp, Artificial Sequence
  Synthetic Construct
agtgtcaagtccaatctatgattcaagagatcatagattggacttgacacttt

SEQ ID NO: 9 - DNA, 53 bp, Artificial Sequence
  Synthetic Construct
agugucaaguccaaucuaugauucaagagaucauagauuggacuugacacuuu

SEQ ID NO: 10 - DNA, 53 bp, Artificial Sequence
  Synthetic Construct
gcggagacagcgacgaagagcuuuguguaggcucuucgucgcugucuccgcuu

SEQ ID NO: 11 - DNA, 53 bp, Artificial Sequence
  Synthetic Construct
agugucaaguccaauuugugguucaagagaucauagauuggacuugacacuuu

SEQ ID NO: 12 - DNA, 51 bp, Artificial Sequence
  Synthetic Construct
gcacugaauagaaauggugguucaagagaucacuauuucuauucagugcuu

SEQ ID NO: 13 - protein, 104 aa, Artificial Sequence
  Synthetic Construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTV

SEQ ID NO: 14 - protein, 132 aa, Artificial Sequence
  Synthetic Construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 15 - protein, 105 aa, Artificial Sequence
  Synthetic Construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVL

SEQ ID NO: 16 - protein, 135 aa, Artificial Sequence
  Synthetic Construct
QPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSS

SEQ ID NO: 17 - protein, 110 aa, Artificial Sequence
  Synthetic Construct
QSVLTQPASVSASPGQSITVSCTGSRNDVGGYDFVSWYQRHPGGVPKLIIYEISKRPSGIPQRFSGSRSGNTASLTISGLQDDDEADYYCCSYASYDRLIFGGGTRVSVL

SEQ ID NO: 18 - protein, 128 aa, Artificial Sequence
  Synthetic Construct
QVQLMQSGAQLRDPGDSLKISCKASGYNFIDYHIHWVRLAPGRGLEWMGWIDPVGGITKYAGQFQGRLSLTRDTSTNTLFLELSRLTAGDTAVYFCARSMRPVDHGIDYSGLFVFHFWGRGSDVLVSS